(19) United States
(12) Patent Application Publication
(10) Pub. No.: US 2025/0304722 A1
(43) Pub. Date: Oct. 2, 2025

(54) ANTI-NALOXONE AND ANTI-NALTREXONE MONOCLONAL ANTIBODIES AND METHODS OF PRODUCTION AND USE THEREOF

(71) Applicant: Siemens Healthcare Diagnostics Inc., Tarrytown, NY (US)

(72) Inventors: Manoj Sharma, Hockessin, DE (US); Tatiana Lebedeva, Aston, PA (US); Tatiana Mareeva, Haddonfield, NJ (US); Yuriy Smirnov, Haddonfield, NJ (US); Bogdan Draghici, Newark, DE (US); Zhu Teng, Garnet Valley, PA (US); Yi Feng Zheng, Wilmington, DE (US); Heungyeung Yau, Wilmington, DE (US); Nihmat Morjana, Newark, DE (US); William Bedzyk, Odessa, DE (US); Allison Dominowski, Middletown, DE (US)

(73) Assignee: Siemens Healthcare Diagnostics Inc., Tarrytown, NY (US)

(21) Appl. No.: 19/238,636

(22) Filed: Jun. 16, 2025

Related U.S. Application Data
(62) Division of application No. 17/310,042, filed on Jul. 13, 2021.
(60) Provisional application No. 62/799,399, filed on Jan. 31, 2019.
(60) Provisional application No. 62/820,400, filed on Mar. 19, 2019.

(51) Int. Cl. C07K 16/44; A61K 47/64; A61P 25/36; G01N 33/94

(52) U.S. Cl. CPC C07K 16/44; A61K 47/643; A61P 25/36; C07K 2317/10; C07K 2317/565; G01N 33/94

(57) ABSTRACT

Antibodies that specifically bind naloxone and/or naltrexone are disclosed. Also disclosed are conjugates used in the production of the antibodies, as well as methods of producing the antibodies. Also disclosed are methods of using the antibodies in direct assays for naloxone and/or naltrexone. Further disclosed are methods of reducing naloxone and/or naltrexone interference in opiate assays using the antibodies.

### CROSS REFERENCE TO RELATED APPLICATIONS/INCORPORATION BY REFERENCE STATEMENT

This is a divisional application claiming priority from U.S. Ser. No. 17/310,042 filed Jul. 13, 2021 which is a National Stage of PCT/US2020/015773 filed Jan. 30, 2020 which claims priority to U.S. Provisional Patent Application Nos. 62/799,399 filed Jan. 31, 2019 and 62/820,400 filed Mar. 19, 2019, all of which are hereby incorporated by reference herein.

This application incorporates by reference the sequence listing which is submitted together with this application in computer readable form which has the file name 2019P02378SL.xml created on Apr. 3, 2025 and is 359000 bytes.

### STATEMENT REGARDING FEDERALLY SPONSORED RESEARCH OR DEVELOPMENT

Not Applicable.

### BACKGROUND

Naloxone is commonly used as an antagonist of abused opioids, typically in drug overdose cases. Naloxone is a life-saving, short acting drug that will bring a patient out of an opiate overdose by stripping the opiate from the opiate receptor. Patients are given repeated shots of naloxone (NARCAN® (naloxone HCl), ADAPT Pharma, Inc., Radnor, PA.) to saturate drug receptors so that the pharmacological response to abused opioids is minimized. Naloxone acts within minutes and lasts for about an hour.

Naltrexone is another commonly used opioid antagonist drug that is also used in alcohol abuse cases; naltrexone is an opiate/alcohol blocking agent that has been used for the last 30 years. Naltrexone acts slowly and lasts longer than naloxone. VIVITROL® (Alkermes, Inc., Dublin, Ireland) is an extended release form of naltrexone. The extended release properties of VIVITROL® assist with patient compliance, since daily use is not required.

Both naloxone and naltrexone will also blunt the effects of alcohol, and as such, they are often used after discharge from a residential treatment program to prevent alcohol relapse.

Naloxone has a rapid distribution throughout the body. The mean serum half-life has been shown to range from 30 to 81 minutes, which is shorter than the average half-life of some opiates, and which necessitates repeat dosing if opioid receptors must be stopped from triggering for an extended period of time. Naloxone is primarily metabolized by the liver; its major metabolite is naloxone-3-glucuronide, which is excreted in the urine.

Naltrexone is metabolized in the liver mainly to 6β-naltrexol by the enzyme dihydrodiol dehydrogenase. Other metabolites include 2-hydroxy-3-methoxy-6β-naltrexol and 2-hydroxy-3-methoxy-naltrexone. These intermediates are then further metabolized by conjugation with glucuronide. The plasma half-life of naltrexone and its metabolite 6β-naltrexol are about four (4) and thirteen (13) hours, respectively.

Randox Toxicology (Crumlin, United Kingdom) offers a naloxone assay based on BioChip Array Technology (BAT) that is based on Enzyme-Linked Immunosorbent Assay (ELISA) principles. Biochip Array Technology is a precision multiplex testing platform allowing for the simultaneous quantitative or qualitative detection of a wide range of analytes from a single sample. The Biochip is a solid-state device with discrete test sites onto which antibodies specific to different drug compounds are immobilized and stabilized. Competitive chemiluminescent immunoassays are then employed, offering a highly sensitive screen. However, for this assay, the level of naltrexone cross-reactivity is 12.5%, and the level of naloxone 3-B-D glucuronide cross-reactivity is 70.6%.

Immunalysis Corporation (Pomona, CA) offers an ELISA Naltrexone assay for oral fluid and forensic use and also based on ELISA principles (see, for example, catalog numbers 239-0096 and 239-0480).

Neogen Corporation (Lansing, MI) offers a Naltrexone/Nalbuphine ELISA kit that is a qualitative one-step kit designed for use as a screening device for the detection of naltrexone, nalbuphine, and/or other metabolites. This test, however, has only 4% cross-reactivity to naloxone (see, for example, catalog numbers 133015 and 133019).

Other methods of detecting naloxone and naltrexone involve High Performance Liquid Chromatography/Tandem Mass Spectrometry (LC-MS/MS) analysis. For example, NMS Labs (Willow Grove, PA) offer LC-MS/MS analyses for naltrexone (total) and the metabolite 6-beta-naltrexol (total) in urine (see, for example, Test Code 3116U, Test Name Naltrexone and Metabolite-Total (Conjugated/Unconjugated), Urine).

However, there are currently no known homogeneous immunoassays available to either or both of these two drugs. Thus, there is a need in the art for monoclonal antibodies that specifically bind to naloxone and/or naltrexone, as well as homogeneous immunoassays that can be utilized to detect the presence of these drugs in a biological sample and/or provide a qualitative screening and/or a quantitative clinical measurement thereof.

In addition, given the similar structure of naloxone and naltrexone to various opiates (see FIG. 1), these drugs can cross-react in various opiate assays. In particular, it is commonly observed that various opiate assays provide false positive results for patients undergoing naloxone/naltrexone treatment. Thus, there is also a need in the art to neutralize naloxone and naltrexone present in biological samples to thereby prevent any false positive or falsely elevated opiate measurements, thus providing more accurate qualitative screenings and quantitative measurements in clinical opiate assays.

### BRIEF DESCRIPTION OF THE DRAWINGS

FIG. 1 depicts the chemical structures of naloxone and naltrexone as well as of various opiates with which naloxone and naltrexone cross-react in various opiate assays.

FIG. 2 schematically depicts syntheses of naltrexone hapten, KLH immunogen, and ovalbumin and G6PDH conjugates through C-3 position linkage.

FIG. 3 schematically depicts syntheses of naloxone hapten, KLH immunogen, and ovalbumin and G6PDH conjugates through C-3 position linkage.

FIG. 4 schematically depicts syntheses of naltrexone hapten, KLH immunogen, and ovalbumin and G6PDH conjugates through C-6 position linkage.

FIG. 5 schematically depicts syntheses of naloxone hapten, KLH immunogen, and ovalbumin and G6PDH conjugates through C-6 position linkage.

FIG. 6 graphically depicts ELISA inhibition assays for various opiates conducted using various bleeds obtained from selected mice immunized with naloxone- or naltrexone-KLH conjugates.

FIG. 7 graphically depicts ELISA inhibition assays for five selected anti-naloxone monoclonal antibodies using eight key opioids (immunogen indicated in lower right of Figure).

FIG. 8 graphically depicts ELISA inhibition assays for eight selected anti-naltrexone monoclonal antibodies using eight key opioids (immunogen indicated in lower panel of Figure).

FIG. 9 graphically depicts a combination naloxone and naltrexone specific assay using the monoclonal antibody 179A 6H6 (produced using a naloxone-containing immunogen).

FIG. 10 graphically depicts a combination naloxone and naltrexone specific assay using the monoclonal antibody 180C 2A12 (produced using a naltrexone-containing immunogen).

FIG. 11 illustrates an evaluation of the monoclonal antibody 180A 3D3 in an opiate enzyme multiplied immunoassay.

FIG. 12 illustrates an evaluation of the monoclonal antibody 179A 6H6 in an opiate enzyme multiplied immunoassay.

FIG. 13 illustrates an evaluation of the monoclonal antibody 180C 2A12 in an opiate enzyme multiplied immunoassay.

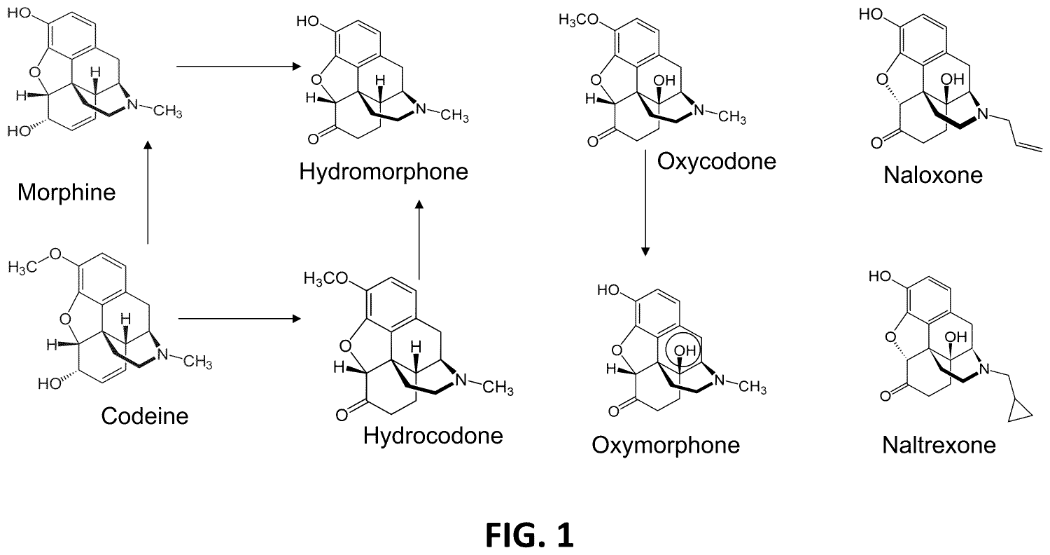

### DETAILED DESCRIPTION

Before explaining at least one embodiment of the present disclosure in detail by way of exemplary language and results, it is to be understood that the present disclosure is not limited in its application to the details of construction and the arrangement of the components set forth in the following description. The present disclosure is capable of other embodiments or of being practiced or carried out in various ways. As such, the language used herein is intended to be given the broadest possible scope and meaning; and the embodiments are meant to be exemplary-not exhaustive. Also, it is to be understood that the phraseology and terminology employed herein is for the purpose of description and should not be regarded as limiting.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2^{nd} ed., Cold Spring Harbor Laboratory Press, Cold Spring Harbor, N.Y. (1989) and Coligan et al. Current Protocols in Immunology (Current Protocols, Wiley Interscience (1994)), which are incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions, kits, and/or methods disclosed herein can be made and executed without undue experimentation in light of the present disclosure. While the compositions, kits, and/or methods have been described in terms of particular embodiments, it will be apparent to those of skill in the art that variations may be applied to the compositions, kits, and/or methods and in the steps or in the sequence of steps of the methods described herein without departing from the concept, spirit, and scope of the present disclosure. All such similar substitutes and modifications apparent to those skilled in the art are deemed to be within the spirit, scope, and concept of the present disclosure as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. The term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

The terms “analog” and “derivative” are used herein interchangeably and refer to a substance which comprises the same basic carbon skeleton and carbon functionality in its structure as a given compound, but can also contain one or more substitutions thereto. The term “substitution” as used herein will be understood to refer to the replacement of at least one substituent on a compound with a residue R. In certain non-limiting embodiments, R may include H, hydroxyl, thiol, a halogenid selected from fluoride, chloride bromide or iodite, a C1-C4 compound selected one of the following: linear, branched or cyclic alkyl, optionally substituted, and linear branched or cyclic alkenyl, wherein the optional substitutents are selected from one or more alkenylalkyl, alkynylalkyl, cycloalkyl, cycloalkenylalkyl, arylalkyl, heteroarylalkyl, heterocyclealkyl, optionally substituted heterocycloalkenylalkyl, arylcycloalkyl, and arylheterocycloalkyl, each of which is optionally substituted wherein the optional substitutents are selected from one or more of alkenylalkyl, alkynylalkyl, cycloalkyl, cyclalkenylalkyl, arylalkyl, alkylaryl, heteroarylalkyl, heterocyclealkyl, optionally substituted heterocycloalkenylalkyl, arylcycloalkyl, and arylheterocyclalkyl, phenyl, cyano, hydroxyl, alkyl, aryl, cycloalkyl, cyano, alkoxy, alkylthio, amino, —NH (alkyl), —NH(cycloalkyl)₂, carboxy, and —C(O))-alkyl.

The term “sample” as used herein will be understood to include any type of biological sample that may be utilized in accordance with the present disclosure. Examples of fluidic biological samples that may be utilized include, but are not limited to, whole blood or any portion thereof (i.e., plasma or serum), urine, saliva, sputum, cerebrospinal fluid (CSF), skin, intestinal fluid, intraperitoneal fluid, cystic fluid, sweat, interstitial fluid, extracellular fluid, tears, mucus, bladder wash, semen, fecal, pleural fluid, nasopharyngeal fluid, combinations thereof, and the like.

The term “specific binding partner,” as used herein, will be understood to refer to any molecule capable of specifically associating with a macrophilin-binding pharmaceutical for purposes of detection thereof. For example, but not by way of limitation, the specific binding partner may be an antibody, a receptor, a ligand, an aptamer, a molecular imprinted polymer (i.e., inorganic matrices), or any combination and/or derivative(s) thereof, as well as any other molecules capable of specific binding to the macrophilin-binding pharmaceutical.

The term “antibody” is used herein in the broadest sense and refers to, for example, intact monoclonal antibodies and polyclonal antibodies, multi-specific antibodies (e.g., bispecific antibodies), antibody fragments and conjugates thereof that exhibit the desired biological activity of analyte binding (such as, but not limited to, Fab, Fab′, F(ab′)2, Fv, scFv, Fd, diabodies, single-chain antibodies, and other antibody fragments and conjugates thereof that retain at least a portion of the variable region of an intact antibody), antibody substitute proteins or peptides (i.e., engineered binding proteins/peptides), and combinations or derivatives thereof. The antibody can be of any type or class (e.g., IgG, IgE, IgM, IgD, and IgA) or sub-class (e.g., IgG1, IgG2, IgG2a, IgG2b, IgG3, IgG4, IgA1, and IgA2).

The terms “peptide,” “polypeptide,” and “protein” are used herein to refer to a polymer of amino acid residues. The term “polypeptide” as used herein is a generic term to refer to native protein, protein fragments, or analogs of a polypeptide sequence. Hence, native protein, protein fragments, and analogs are species of the polypeptide genus. The term “isolated peptide/polypeptide/protein” as used herein refers to a peptide/polypeptide/protein of cDNA, recombinant RNA, or synthetic origin or some combination thereof, which by virtue of its origin, or source of derivation, the “isolated peptide/polypeptide/protein”: (1) is not associated with peptides/polypeptides/proteins found in nature, (2) is free of other peptides/polypeptides/proteins from the same source, e.g., free of murine proteins, (3) is expressed by a cell from a different species, and/or (4) does not occur in nature.

As used herein, the term “amino acid” embraces all molecules, whether natural or synthetic, which include both an amino functionality and an acid functionality and capable of being included in a polymer of naturally-occurring amino acids. Exemplary amino acids include naturally-occurring amino acids; analogs, derivatives, and congeners thereof; amino acid analogs having variant side chains; and all stereoisomers of any of any of the foregoing.

The terms “polynucleotide” and “nucleic acid” are used interchangeably. They refer to a polymeric form of nucleotides of any length, either deoxyribonucleotides or ribonucleotides, or analogs thereof. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component. The terms “isolated nucleic acid” and “isolated polynucleotide” are used interchangeably; a nucleic acid or polynucleotide is considered “isolated” if it: (1) is not associated with all or a portion of a polynucleotide in which the “isolated polynucleotide” is found in nature, (2) is linked to a polynucleotide to which it is not linked in nature, or (3) does not occur in nature as part of a larger sequence.

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby be replicated along with the host genome. Moreover, certain vectors are capable of directing the expression of genes. Such vectors are referred to herein as “recombinant expression vectors” (or simply, “expression vectors”).

The term “naturally-occurring” as used herein as applied to an object refers to the fact that an object can be found in nature. For example, a polynucleotide or polypeptide sequence that is present in an organism (including viruses) that can be isolated from a source in nature and which has not been intentionally modified by man in the laboratory or otherwise is naturally-occurring. The term “naturally-occurring” may be used interchangeably herein with the term “native.”

The term “selectively hybridize” referred to herein means to detectably and specifically bind. Polynucleotides, oligonucleotides and fragments thereof encoding peptides/polypeptides/proteins in accordance with the inventive concept(s) selectively hybridize to nucleic acid strands under hybridization and wash conditions that minimize appreciable amounts of detectable binding to nonspecific nucleic acids. High stringency conditions can be used to achieve selective hybridization conditions as known in the art and discussed herein. Generally, the nucleic acid sequence homology between the polynucleotides, oligonucleotides, and fragments of the inventive concept(s) and a nucleic acid sequence of interest will be at least 80%, and more typically with increasing homologies of at least 85%, 90%, 95%, 99%, and 100%. Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred (but non-limiting), with 2 or less being more preferred (but non-limiting). Alternatively, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See Dayhoff, M. O., in Atlas of Protein Sequence and Structure, pp. 101-110 (Volume 5, National Biomedical Research Foundation (1972)) and Supplement 2 to this volume, pp. 1-10. The two sequences or parts thereof are more preferably homologous if their amino acids are greater than or equal to 50% identical when optimally aligned using the ALIGN program. The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA.”

The following terms are used to describe the sequence relationships between two or more polynucleotide or amino acid sequences: “reference sequence,” “comparison window,” “sequence identity,” “percentage of sequence identity,” and “substantial identity.” A “reference sequence” is a defined sequence used as a basis for a sequence comparison; a reference sequence may be a subset of a larger sequence, for example, as a segment of a full-length cDNA or gene sequence given in a sequence listing or may comprise a complete cDNA or gene sequence. Generally, a reference sequence is at least 18 nucleotides or 6 amino acids in length, frequently at least 24 nucleotides or 8 amino acids in length, and often at least 48 nucleotides or 16 amino acids in length. Since two polynucleotides or amino acid sequences may each (1) comprise a sequence (i.e., a portion of the complete polynucleotide or amino acid sequence) that is similar between the two molecules, and (2) may further comprise a sequence that is divergent between the two polynucleotides or amino acid sequences, sequence comparisons between two (or more) molecules are typically performed by comparing sequences of the two molecules over a “comparison window” to identify and compare local regions of sequence similarity. A “comparison window,” as used herein, refers to a conceptual segment of at least 18 contiguous nucleotide positions or 6 amino acids wherein a polynucleotide sequence or amino acid sequence may be compared to a reference sequence of at least 18 contiguous nucleotides or 6 amino acid sequences and wherein the portion of the polynucleotide sequence in the comparison window may comprise additions, deletions, substitutions, and the like (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman (Adv. Appl. Math., 2:482 (1981)), by the homology alignment algorithm of Needleman and Wunsch (J. Mol. Biol., 48:443 (1970)), by the search for similarity method of Pearson and Lipman (Proc. Natl. Acad. Sci. (U.S.A.), 85:2444 (1988)), by computerized implementations of these algorithms (GAP, BESTFIT, FASTA, and TFASTA in the Wisconsin Genetics Software Package Release 7.0, (Genetics Computer Group, 575 Science Dr., Madison, Wis.), Geneworks, or MacVector software packages, or by inspection, and the best alignment (i.e., resulting in the highest percentage of homology over the comparison window) generated by the various methods is selected.

The term “sequence identity” means that two polynucleotide or amino acid sequences is identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or I) or residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, such as at least 90 to 95 percent sequence identity, or at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As used herein, the twenty conventional amino acids and their abbreviations follow conventional usage. See Immunology—A Synthesis (2nd Edition, E. S. Golub and D. R. Gren, Eds., Sinauer Associates, Sunderland, Mass. (1991)), which is incorporated herein by reference. Stereoisomers (e.g., D-amino acids) of the twenty conventional amino acids, unnatural amino acids such as α-,α-disubstituted amino acids, N-alkyl amino acids, lactic acid, and other unconventional amino acids may also be suitable components for polypeptides of the present disclosure. Examples of unconventional amino acids include: 4-hydroxyproline, α-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

As applied to polypeptides, the term “substantial identity” means that two peptide sequences, when optimally aligned, such as by the programs GAP or BESTFIT using default gap weights, share at least 80 percent sequence identity, such as at least 90 percent sequence identity, or at least 95 percent sequence identity, or at least 99 percent sequence identity. In certain particular (but non-limiting) embodiments, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Particular conservative amino acids substitution groups are: valine-leucine-isoleucine, phenylalanine-tyrosine, lysine-arginine, alanine-valine, glutamic-aspartic, and asparagine-glutamine.

The term “variant” of a reference polypeptide refers to a polypeptide having one or more amino acid substitutions, deletions or insertions relative to the reference polypeptide. An amino acid substitution may be “conservative” or “non-conservative.” A “conservative” amino acid substitution refers to the substitution of an amino acid in a polypeptide with another amino acid having similar properties, such as but not limited to, size and charge. Conservative replacements are those that take place within a family of amino acids that are related in their side chains. Genetically encoded amino acids are generally divided into families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. More particular families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. In particular (but non-limiting) embodiments, computerized comparison methods are used to identify sequence motifs or predicted protein conformation domains that occur in other proteins of known structure and/or function. Methods to identify protein sequences that fold into a known three-dimensional structure are known (Bowie et al., Science, 253:164 (1991)). Thus, the foregoing examples demonstrate that those of skill in the art can recognize sequence motifs and structural conformations that may be used to define structural and functional domains in accordance with the present disclosure.

Preferred amino acid substitutions are those which: (1) reduce susceptibility to proteolysis, (2) reduce susceptibility to oxidation, (3) alter binding affinity for forming protein complexes, (4) alter binding affinities, and (5) confer or modify other physicochemical or functional properties of such analogs. Analogs can include various mutations of a sequence other than the naturally-occurring peptide sequence. For example, single or multiple amino acid substitutions (such as, but not limited to, conservative amino acid substitutions) may be made in the naturally-occurring sequence (such as, but not limited to, in the portion of the polypeptide outside the domain(s) forming intermolecular contacts). A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure© (Branden and J. Tooze, eds., Garland Publishing, New York, N.Y. (1991)); and Thornton et al. (Nature 354:105 (1991)), which are each incorporated herein by reference.

The term “polypeptide fragment” as used herein refers to a polypeptide that has an amino-terminal and/or carboxy-terminal deletion, but where the remaining amino acid sequence is identical to the corresponding positions in the naturally-occurring sequence. A polypeptide fragment may be any length that is less than the length of the reference polypeptide.

The term “antibody” is used in the broadest sense, and specifically covers monoclonal antibodies (including full length monoclonal antibodies), polyclonal antibodies, multispecific antibodies (e.g., bispecific antibodies), and antibody fragments so long as they exhibit the desired biological activity. Thus, the terms “Antibody” or “antibody peptide(s)” refer to a full length immunoglobulin molecule (i.e., an intact antibody), or a binding fragment thereof that competes with the intact antibody for specific antigen binding. Binding fragments may be produced by recombinant DNA techniques, or by enzymatic or chemical cleavage of intact antibodies. Binding fragments include Fab, Fab′, F(ab′)₂, Fv, scFv, disulfide linked Fv, Fd, diabodies, single-chain antibodies, single domain antibodies (such as but not limited to, NANOBODIES®) and other antibody fragments that retain at least a portion of the variable region of an intact antibody. See, e.g., Hudson et al. (Nature Med., 9:129-134 (2003)).

The term “antigen binding fragment” or “antigen-binding portion” of an antibody, as used herein, refers to one or more fragments of an antibody that retain the ability to bind to an antigen. The antigen-binding function of an antibody can be performed by fragments of an intact antibody. Examples of binding fragments encompassed within the term “antigen-binding fragment” of an antibody include but are not limited to, Fab, Fab′, F(ab′)2, Fv, scFv, disulfide linked Fv, Fd, diabodies, single-chain antibodies, single domain antibodies (such as but not limited to, NANOBODIES®), isolated CDRH3, and other antibody fragments that retain at least a portion of the variable region of an intact antibody. These antibody fragments are obtained using conventional recombinant and/or enzymatic techniques and are screened for antigen binding in the same manner as intact antibodies.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. Kappa and lambda light chains refer to the two major antibody light chain isotypes.

The terms “CDR,” and its plural “CDRs,” refer to a complementarity determining region (CDR) of an antibody or antibody fragment, which determine the binding character of an antibody or antibody fragment. In most instances, three CDRs are present in a light chain variable region (CDRL1, CDRL2 and CDRL3) and three CDRs are present in a heavy chain variable region (CDRH1, CDRH2 and CDRH3). CDRs contribute to the functional activity of an antibody molecule and are separated by amino acid sequences that comprise scaffolding or framework regions. Among the various CDRs, the CDR3 sequences, and particularly CDRH3, are the most diverse and therefore have the strongest contribution to antibody specificity. There are at least two techniques for determining CDRs: (1) an approach based on cross- species sequence variability (i.e., Kabat et al., Sequences of Proteins of Immunological Interest (National Institute of Health, Bethesda, Md. (1987), incorporated by reference in its entirety); and (2) an approach based on crystallographic studies of antigen-antibody complexes (Chothia et al., Nature, 342:877 (1989), incorporated by reference in its entirety).

The term “epitope” includes any protein determinant capable of specific binding to an immunoglobulin or T-cell receptor. In certain embodiments, an epitope is a region of an antigen that is specifically bound by an antibody. Epitopic determinants usually include chemically active surface groupings of molecules such as amino acids, sugar side chains, phosphoryl, or sulfonyl groups. In certain embodiments, an epitope may have specific three-dimensional structural characteristics (e.g., a “conformational epitope”), as well as specific charge characteristics.

An epitope is defined as “the same” as another epitope if a particular antibody specifically binds to both epitopes. In certain embodiments, polypeptides having different primary amino acid sequences may comprise epitopes that are the same. In certain embodiments, epitopes that are the same may have different primary amino acid sequences. Different antibodies are said to bind to the same epitope if they compete for specific binding to that epitope.

An antibody “specifically binds” an antigen when it preferentially recognizes the antigen in a complex mixture of proteins and/or macromolecules. In certain embodiments, an antibody comprises an antigen-binding site that specifically binds to a particular epitope. In certain such embodiments, the antibody is capable of binding different antigens so long as the different antigens comprise that particular epitope or closely related epitopes. In certain instances, for example, homologous proteins from different species may comprise the same epitope. In certain embodiments, an antibody specifically binds to an antigen with a dissociation constant of no greater than 10⁻⁶ M, 10⁻⁷ M, 10⁻⁸ M or 10⁻⁹ M. When an antibody specifically binds to a receptor or ligand (i.e., counterreceptor), it may substantially inhibit adhesion of the receptor to the ligand. As used herein, an antibody substantially inhibits adhesion of a receptor to a ligand when an excess of antibody reduces the quantity of receptor bound to ligand by at least about 20%, 40%, 60% or 80%, 85%, or 90% (as measured in an in vitro competitive binding assay).

An “isolated” antibody is one which has been separated and/or recovered from a component of the environment in which it was produced. Contaminant components of its production environment are materials which would interfere with diagnostic or therapeutic uses for the antibody, and may include enzymes, hormones, and other proteinaceous or nonproteinaceous solutes. In certain embodiments, the antibody will be purified as measurable by at least three different methods: 1) to greater than 50% by weight of antibody as determined by the Lowry method, such as more than 75% by weight, or more than 85% by weight, or more than 95% by weight, or more than 99% by weight; 2) to a degree sufficient to obtain at least 10 residues of N-terminal or internal amino acid sequence by use of a spinning cup sequentator, such as at least 15 residues of sequence; or 3) to homogeneity by SDS-PAGE under reducing or non-reducing conditions using Coomassie blue or, alternatively, silver stain. Isolated antibody includes the antibody in situ within recombinant cells since at least one component of the environment in which the antibody is produced will not be present. Ordinarily, however, isolated antibody will be prepared by at least one purification step. In addition, the “isolated antibody” is substantially free of other antibodies having different antigenic specificities. An isolated antibody may, however, have some cross- reactivity to other, related antigens.

The term “antibody mutant” refers to an amino acid sequence variant of an antibody wherein one or more of the amino acid residues have been modified. Such mutants necessarily have less than 100% sequence identity or similarity with the amino acid sequence having at least 75% amino acid sequence identity or similarity with the amino acid sequence of either the heavy or light chain variable domain of the antibody, such as at least 80%, or at least 85%, or at least 90%, or at least 95%.

The term “monoclonal antibody” as used herein refers to an antibody obtained from a population of substantially homogeneous antibodies that specifically bind to the same epitope, i.e., the individual antibodies comprising the population are identical except for possible naturally occurring mutations that may be present in minor amounts. In contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that in one method of production they may be synthesized by a hybridoma culture, and thus are uncontaminated by other immunoglobulins. The modifier “monoclonal” indicates the character of the antibody as being obtained from a substantially homogeneous population of antibodies, and is not to be construed as requiring production of the antibody by any particular method. For example, in one embodiment, the monoclonal antibodies produced in accordance with the present disclosure may be made by the hybridoma method first described by Kohler and Milstein (Nature, 256:495 (1975)).

The monoclonal antibodies utilized in accordance with the present disclosure may be produced by any methodology known in the art including, but not limited to, a result of a deliberate immunization protocol; a result of an immune response that results in the production of antibodies naturally in the course of a disease or cancer; phage-derived antibodies; and the like. In addition to the hybridoma production method listed above, the monoclonal antibodies of the present disclosure may be produced by other various methods such as, but not limited to, recombinant DNA methods (see, e.g., U.S. Pat. No. 4,816,567); isolation of antibody fragments from a phage display library (see, e.g., Clackson et al., Nature, 352: 624-628 (1991); and Marks et al., J. Mol. Biol., 222:581-597 (1991)); as well as various other monoclonal antibody production techniques (see, e.g., Harlow and Lane (1988) Antibodies: A Laboratory Manual (Cold Spring Harbor Laboratory, Cold Spring Harbor, N.Y.)).

Once the antibodies have been obtained, for example, once individual B cells have been identified and/or monoclonal antibodies have been produced, the sequences encoding the variable regions of these antibodies can be obtained. The variable region sequences can, for example, be obtained by first sequencing the antibody protein produced by the hybridoma, B-cell or phage and determining the encoding nucleic acid sequence. In one embodiment, the immunoglobulin variable region (VH and VL) DNA or cDNA may be sequenced instead. Where the antibody is derived from a hybridoma cell line or isolated B-cell, the cDNAs encoding the variable regions may be amplified using PCR by, for example, the methods described in Babcook et al. (Proc. Natl. Acad. Sci. USA, 93:7843-7848 (1996)), and in PCT Publication No. WO 92/02551. The contents of both references are expressly incorporated herein by reference in their entirety.

The term “neutralizing antibody” or “antibody that neutralizes” refers to an antibody that reduces at least one activity of a polypeptide comprising the epitope to which the antibody specifically binds. In certain embodiments, a neutralizing antibody reduces an activity in vitro and/or in vivo.

The term “antigen-binding site” refers to a portion of an antibody capable of specifically binding an antigen. In certain embodiments, an antigen-binding site is provided by one or more antibody variable regions.

As used herein, “substantially pure” means an object species is the predominant species present (i.e., on a molar basis it is more abundant than any other individual species in the composition). Generally, a substantially pure composition will comprise more than about 50% percent of all macromolecular species present in the composition, such as more than about 55%, 60%, 65%, 70%, 75%, 80%, 85%, 90%, 95%, and 99%. In one embodiment, the object species is purified to essential homogeneity (contaminant species cannot be detected in the composition by conventional detection methods) wherein the composition consists essentially of a single macromolecular species.

The term “agent” refers to a chemical compound, a mixture of chemical compounds, a biological macromolecule, or an extract made from biological materials. In certain embodiments, the “agent” may be a monoclonal antibody in accordance with the present disclosure.

The term “antagonist” refers to an agent that reduces an activity of a protein/enzyme.

The term “agonist” refers to an agent that increases an activity of a protein/enzyme.

The term “carrier protein” as used herein will be understood to refer to an immunogenic protein that can be attached to small molecules (such as (but not limited to) drugs, organic compounds, and peptides and oligosaccharides with a molecular weight of less than 2-5 kDa) that are typically not immunogenic, for the purpose of stimulating an immune system response in the form of production of antibodies against the small molecule.

Turning now to the inventive concepts, certain non-limiting embodiments of the present disclosure are directed to an antibody or functional fragment thereof that specifically binds to naloxone and/or naltrexone. These two compounds are depicted below as Formulas I and II, respectively.

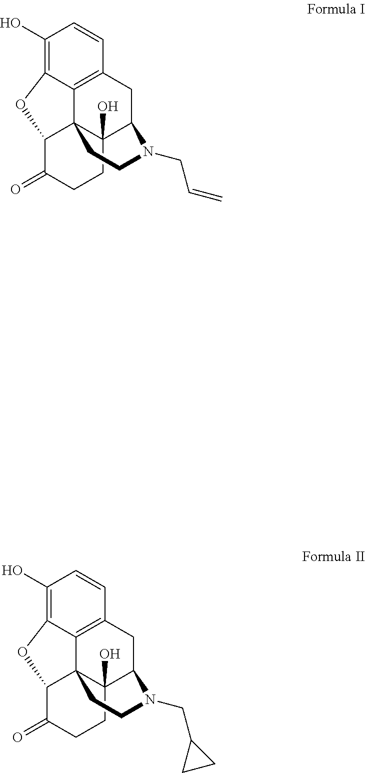

In a particular non-limiting embodiment, the antibody or functional fragment thereof specifically binds to a conjugate comprising naloxone and/or naltrexone bound to a carrier protein. The conjugate may contain any carrier protein known in the art or otherwise contemplated herein, so long as the carrier protein: (1) can be conjugated to naloxone and/or naltrexone; (2) is immunogenic; and (3) can function to stimulate an immune system response in the form of production of antibodies against naloxone and/or naltrexone. Non-limiting examples of carrier proteins that may be utilized in accordance with the present disclosure include KLH (Keyhole Limpet Hemocyanin), BSA (Bovine Serum Albumin), Ovalbumin, BTG (Bovine Thyroglobulin), and BGG (Bovine Gamma Globulin).

In certain non-limiting embodiments, the carrier protein is attached to a specific carbon position on the naloxone and/or naltrexone. For example (but not by way of limitation), the carrier protein may be attached to the 1-carbon, 2-carbon, 3-carbon, 6-carbon, 7-carbon, or 8-carbon position of naloxone and/or naltrexone. Non-limiting examples of these conjugate immunogens are shown in Formulas III-VIII below. Note that each of these formulas covers both naloxone and naltrexone, and that the carrier protein is illustrated as being attached to a different carbon in each formula (Formula III: 3-carbon; Formula IV: 1-carbon; Formula V: 2-carbon; Formula VI: 6-carbon; Formula VII: 8-carbon; and Formula VIII: 7-carbon).

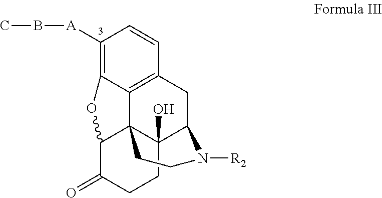

wherein, in Formula III: A is a hetero atom (such as, but not limited to: —O—, —N—, or —S—); B is a functional group (such as, but not limited to: —CO—, a lower alkyl (C₀ to C₁₀), —CONH—, —SO₂—, —PO₄—, and the like); C is a carrier protein (such as, but not limited to: KLH, BSA, OVA, BGG, G6PDH, or poly(amino acid)); and R₂ is H, alkyl, allyl, or methylenecyclopropyl.

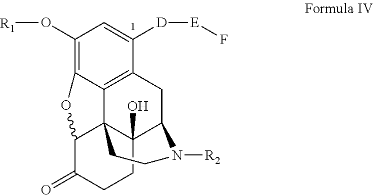

wherein, in Formula IV: D is a hetero atom (such as, but not limited to: O—, S—, CO—, or CH₂—); E is a lower alkyl, CO₂—, CONH—, SO₂—, or PO₄—; F is a carrier protein (such as, but not limited to: KLH, BSA, OVA, BGG, G6PDH, or poly (amino acid)); R₂ is H, alkyl, allyl, or methylenecyclopropyl; and R₁ is H or CH₃.

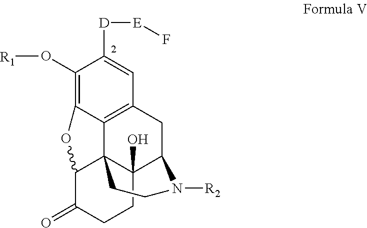

wherein, in Formula V: D is a hetero atom (such as, but not limited to: O—, S—, CO—, or CH₂—); E is a lower alkyl, CO₂—, CONH—, SO₂—, or PO₄—; F is a carrier protein (such as, but not limited to: KLH, BSA, OVA, BGG, G6PDH, or poly (amino acid)); R₂ is H, alkyl, allyl, or methylenecyclopropyl; and R₁ is H or CH₃.

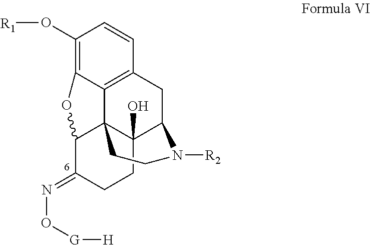

wherein, in Formula VI: G is a linking group which includes at least two of the following: a lower alkyl chain, CO₂—, CONH—, SO₂—, and PO₄—; H is a carrier protein (such as, but not limited to: KLH, BSA, OVA, BGG, G6PDH, or poly (amino acid)); R₁ is H or CH₃; and R₂ is H, alkyl, allyl, or methylenecyclopropyl.

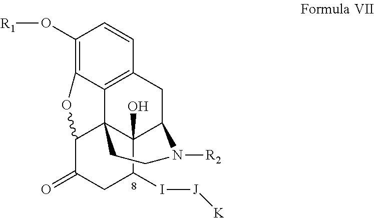

wherein, in Formula VII: I is a hetero atom (such as, but not limited to: O—, S—, CO—, or CH₂—); J is a lower alkyl chain, CO₂—, CONH—, SO₂—, or PO₄—; K is a carrier protein (such as, but not limited to: KLH, BSA, OVA, BGG, G6PDH, or poly (amino acid)); R₁ is H or CH₃; and R₂ is H, alkyl, allyl, or methylenecyclopropyl.

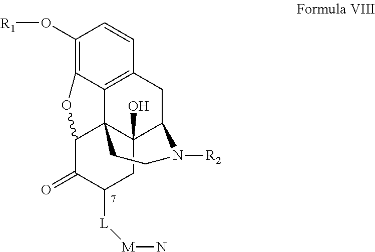

wherein, in Formula VII: L is a hetero atom (such as, but not limited to O, S, CO, or CH₂); M is a lower alkyl chain, CO₂—, CONH—, SO₂—, or PO₄—; N is a carrier protein (such as, but not limited to: KLH, BSA, OVA, BGG, G6PDH, and the like); R₁ is H or CH₃; and R₂ is H, alkyl, allyl, or methylenecyclopropyl.

In a particular (but non-limiting) embodiment, the antibody or functional fragment thereof specifically binds to a conjugate comprising OVA attached to the 3-carbon position and/or 6-carbon position of naloxone and/or naltrexone.

Certain non-limiting embodiments of the present disclosure are directed to an antibody or functional fragment thereof that specifically binds to naltrexone (and in certain particular (but non-limiting) embodiments, the conjugate of Formula III, wherein R₂ is an allyl), wherein the antibody comprises one or more of: (i) a heavy chain variable region CDR1 having the amino acid sequence of SEQ ID NO:2; (ii) a heavy chain variable region CDR2 having the amino acid sequence of SEQ ID NO:3; (iii) a heavy chain variable region CDR3 having the amino acid sequence of SEQ ID NO:4; (iv) a light chain variable region CDR1 having the amino acid sequence of SEQ ID NO:6; (v) a light chain variable region CDR2 having the amino acid sequence of SEQ ID NO:7; and (vi) a light chain variable region CDR3 having the amino acid sequence of SEQ ID NO:8. In a particular non-limiting embodiment, the antibody or functional fragment thereof comprises two, three, four, or five of (i)-(vi) above. In another particular non-limiting embodiment, the antibody or functional fragment thereof comprises all of (i)-(vi) above.

Certain non-limiting embodiments of the present disclosure are directed to an antibody or functional fragment thereof that specifically binds to naltrexone, wherein the antibody comprises one or more of: (i) a heavy chain variable region CDR1 having the amino acid sequence of SEQ ID NO:22; (ii) a heavy chain variable region CDR2 having the amino acid sequence of SEQ ID NO:23; (iii) a heavy chain variable region CDR3 having the amino acid sequence of SEQ ID NO:24; (iv) a light chain variable region CDR1 having the amino acid sequence of SEQ ID NO:26; (v) a light chain variable region CDR2 having the amino acid sequence of SEQ ID NO:27; and (vi) a light chain variable region CDR3 having the amino acid sequence of SEQ ID NO:28. In a particular non-limiting embodiment, the antibody or functional fragment thereof comprises two, three, four, or five of (i)-(vi) above. In another particular non-limiting embodiment, the antibody or functional fragment thereof comprises all of (i)-(vi) above.

Certain non-limiting embodiments of the present disclosure are directed to an antibody or functional fragment thereof that specifically binds to naltrexone, wherein the antibody comprises one or more of: (i) a heavy chain variable region CDR1 having the amino acid sequence of SEQ ID NO:42; (ii) a heavy chain variable region CDR2 having the amino acid sequence of SEQ ID NO:43; (iii) a heavy chain variable region CDR3 having the amino acid sequence of SEQ ID NO:44; (iv) a light chain variable region CDR1 having the amino acid sequence of SEQ ID NO:46; (v) a light chain variable region CDR2 having the amino acid sequence of SEQ ID NO:47; and (vi) a light chain variable region CDR3 having the amino acid sequence of SEQ ID NO:48. In a particular non-limiting embodiment, the antibody or functional fragment thereof comprises two, three, four, or five of (i)-(vi) above. In another particular non-limiting embodiment, the antibody or functional fragment thereof comprises all of (i)-(vi) above.

Certain non-limiting embodiments of the present disclosure are directed to an antibody or functional fragment thereof that specifically binds to naltrexone, wherein the antibody comprises one or more of: (i) a heavy chain variable region CDR1 having the amino acid sequence of SEQ ID NO:62; (ii) a heavy chain variable region CDR2 having the amino acid sequence of SEQ ID NO:63; (iii) a heavy chain variable region CDR3 having the amino acid sequence of SEQ ID NO:64; (iv) a light chain variable region CDR1 having the amino acid sequence of SEQ ID NO:66; (v) a light chain variable region CDR2 having the amino acid sequence of SEQ ID NO:67; and (vi) a light chain variable region CDR3 having the amino acid sequence of SEQ ID NO:68. In a particular non-limiting embodiment, the antibody or functional fragment thereof comprises two, three, four, or five of (i)-(vi) above. In another particular non-limiting embodiment, the antibody or functional fragment thereof comprises all of (i)-(vi) above.

Certain non-limiting embodiments of the present disclosure are directed to an antibody or functional fragment thereof that specifically binds to naltrexone, wherein the antibody comprises one or more of: (i) a heavy chain variable region CDR1 having the amino acid sequence of SEQ ID NO:82; (ii) a heavy chain variable region CDR2 having the amino acid sequence of SEQ ID NO:83; (iii) a heavy chain variable region CDR3 having the amino acid sequence of SEQ ID NO:84; (iv) a light chain variable region CDR1 having the amino acid sequence of SEQ ID NO:86; (v) a light chain variable region CDR2 having the amino acid sequence of SEQ ID NO:87; and (vi) a light chain variable region CDR3 having the amino acid sequence of SEQ ID NO:88. In a particular non-limiting embodiment, the antibody or functional fragment thereof comprises two, three, four, or five of (i)-(vi) above. In another particular non-limiting embodiment, the antibody or functional fragment thereof comprises all of (i)-(vi) above.

Certain non-limiting embodiments of the present disclosure are directed to an antibody or functional fragment thereof that specifically binds to naltrexone, wherein the antibody comprises one or more of: (i) a heavy chain variable region CDR1 having the amino acid sequence of SEQ ID NO:102; (ii) a heavy chain variable region CDR2 having the amino acid sequence of SEQ ID NO:103; (iii) a heavy chain variable region CDR3 having the amino acid sequence of SEQ ID NO:104; (iv) a light chain variable region CDR1 having the amino acid sequence of SEQ ID NO:106; (v) a light chain variable region CDR2 having the amino acid sequence of SEQ ID NO:107; and (vi) a light chain variable region CDR3 having the amino acid sequence of SEQ ID NO:108. In a particular non-limiting embodiment, the antibody or functional fragment thereof comprises two, three, four, or five of (i)-(vi) above. In another particular non-limiting embodiment, the antibody or functional fragment thereof comprises all of (i)-(vi) above.

Certain non-limiting embodiments of the present disclosure are directed to an antibody or functional fragment thereof that specifically binds to naltrexone, wherein the antibody comprises one or more of: (i) a heavy chain variable region CDR1 having the amino acid sequence of SEQ ID NO:122; (ii) a heavy chain variable region CDR2 having the amino acid sequence of SEQ ID NO:123; (iii) a heavy chain variable region CDR3 having the amino acid sequence of SEQ ID NO:124; (iv) a light chain variable region CDR1 having the amino acid sequence of SEQ ID NO:126; (v) a light chain variable region CDR2 having the amino acid sequence of SEQ ID NO:127; and (vi) a light chain variable region CDR3 having the amino acid sequence of SEQ ID NO:128. In a particular non-limiting embodiment, the antibody or functional fragment thereof comprises two, three, four, or five of (i)-(vi) above. In another particular non-limiting embodiment, the antibody or functional fragment thereof comprises all of (i)-(vi) above.

Certain non-limiting embodiments of the present disclosure are directed to an antibody or functional fragment thereof that specifically binds to naltrexone, wherein the antibody comprises one or more of: (i) a heavy chain variable region CDR1 having the amino acid sequence of SEQ ID NO:142; (ii) a heavy chain variable region CDR2 having the amino acid sequence of SEQ ID NO:143; (iii) a heavy chain variable region CDR3 having the amino acid sequence of SEQ ID NO:144; (iv) a light chain variable region CDR1 having the amino acid sequence of SEQ ID NO:146; (v) a light chain variable region CDR2 having the amino acid sequence of SEQ ID NO:147; and (vi) a light chain variable region CDR3 having the amino acid sequence of SEQ ID NO:148. In a particular non-limiting embodiment, the antibody or functional fragment thereof comprises two, three, four, or five of (i)-(vi) above. In another particular non-limiting embodiment, the antibody or functional fragment thereof comprises all of (i)-(vi) above.

Certain non-limiting embodiments of the present disclosure are directed to an antibody or functional fragment thereof that specifically binds to naloxone, wherein the antibody comprises one or more of: (i) a heavy chain variable region CDR1 having the amino acid sequence of SEQ ID NO:162; (ii) a heavy chain variable region CDR2 having the amino acid sequence of SEQ ID NO:163; (iii) a heavy chain variable region CDR3 having the amino acid sequence of SEQ ID NO:164; (iv) a light chain variable region CDR1 having the amino acid sequence of SEQ ID NO:166; (v) a light chain variable region CDR2 having the amino acid sequence of SEQ ID NO:167; and (vi) a light chain variable region CDR3 having the amino acid sequence of SEQ ID NO:168. In a particular non-limiting embodiment, the antibody or functional fragment thereof comprises two, three, four, or five of (i)-(vi) above. In another particular non-limiting embodiment, the antibody or functional fragment thereof comprises all of (i)-(vi) above.

Certain non-limiting embodiments of the present disclosure are directed to an antibody or functional fragment thereof that specifically binds to naloxone, wherein the antibody comprises one or more of: (i) a heavy chain variable region CDR1 having the amino acid sequence of SEQ ID NO:182; (ii) a heavy chain variable region CDR2 having the amino acid sequence of SEQ ID NO:183; (iii) a heavy chain variable region CDR3 having the amino acid sequence of SEQ ID NO:184; (iv) a light chain variable region CDR1 having the amino acid sequence of SEQ ID NO:186; (v) a light chain variable region CDR2 having the amino acid sequence of SEQ ID NO:187; and (vi) a light chain variable region CDR3 having the amino acid sequence of SEQ ID NO:188. In a particular non-limiting embodiment, the antibody or functional fragment thereof comprises two, three, four, or five of (i)-(vi) above. In another particular non-limiting embodiment, the antibody or functional fragment thereof comprises all of (i)-(vi) above.

Certain non-limiting embodiments of the present disclosure are directed to an antibody or functional fragment thereof that specifically binds to naloxone, wherein the antibody comprises one or more of: (i) a heavy chain variable region CDR1 having the amino acid sequence of SEQ ID NO:202; (ii) a heavy chain variable region CDR2 having the amino acid sequence of SEQ ID NO:203; (iii) a heavy chain variable region CDR3 having the amino acid sequence of SEQ ID NO:204; (iv) a light chain variable region CDR1 having the amino acid sequence of SEQ ID NO:206; (v) a light chain variable region CDR2 having the amino acid sequence of SEQ ID NO:207; and (vi) a light chain variable region CDR3 having the amino acid sequence of SEQ ID NO:208. In a particular non-limiting embodiment, the antibody or functional fragment thereof comprises two, three, four, or five of (i)-(vi) above. In another particular non-limiting embodiment, the antibody or functional fragment thereof comprises all of (i)-(vi) above.

Certain non-limiting embodiments of the present disclosure are directed to an antibody or functional fragment thereof that specifically binds to naloxone, wherein the antibody comprises one or more of: (i) a heavy chain variable region CDR1 having the amino acid sequence of SEQ ID NO:222; (ii) a heavy chain variable region CDR2 having the amino acid sequence of SEQ ID NO:223; (iii) a heavy chain variable region CDR3 having the amino acid sequence of SEQ ID NO:224; (iv) a light chain variable region CDR1 having the amino acid sequence of SEQ ID NO:226; (v) a light chain variable region CDR2 having the amino acid sequence of SEQ ID NO:227; and (vi) a light chain variable region CDR3 having the amino acid sequence of SEQ ID NO:228. In a particular non-limiting embodiment, the antibody or particular non-limiting embodiment, the antibody or functional fragment thereof comprises all of (i)-(vi) above.

Certain non-limiting embodiments of the present disclosure are directed to an antibody or functional fragment thereof that specifically binds to naloxone, wherein the antibody comprises one or more of: (i) a heavy chain variable region CDR1 having the amino acid sequence of SEQ ID NO:242; (ii) a heavy chain variable region CDR2 having the amino acid sequence of SEQ ID NO:243; (iii) a heavy chain variable region CDR3 having the amino acid sequence of SEQ ID NO:244; (iv) a light chain variable region CDR1 having the amino acid sequence of SEQ ID NO:246; (v) a light chain variable region CDR2 having the amino acid sequence of SEQ ID NO:247; and (vi) a light chain variable region CDR3 having the amino acid sequence of SEQ ID NO:248. In a particular non-limiting embodiment, the antibody or functional fragment thereof comprises two, three, four, or five of (i)-(vi) above. In another particular non-limiting embodiment, the antibody or functional fragment thereof comprises all of (i)-(vi) above.

In a particular (but non-limiting) embodiment, the antibody or functional fragment thereof has a heavy chain variable region with an amino acid sequence that is at least about 70% identical to any of the heavy chain variable region amino acid sequences outlined in Tables 2-14 (i.e., SEQ ID NO:17, 37, 57, 77, 97, 117, 137, 157, 177, 197, 217, 237, or 257), such as (but not limited to) at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO:17, 37, 57, 77, 97, 117, 137, 157, 177, 197, 217, 237, or 257.

In a particular (but non-limiting) embodiment, the antibody or functional fragment thereof has a heavy chain variable region with an amino acid sequence that differs from SEQ ID NO:17, 37, 57, 77, 97, 117, 137, 157, 177, 197, 217, 237, or 257 by less than about 25 amino acids, less than about 24 amino acids, less than about 23 amino acids, less than about 22 amino acids, less than about 21 amino acids, less than about 20 amino acids, less than about 19 amino acids, less than about 18 amino acids, less than about 17 amino acids, less than about 16 amino acids, less than about 15 amino acids, less than about 14 amino acids, less than about 13 amino acids, less than about 12 amino acids, less than about 11 amino acids, less than about 10 amino acids, less than about 9 amino acids, less than about 8 amino acids, less than about 7 amino acids, less than about 6 amino acids, less than about 5 amino acids, less than about 4 amino acids, less than about 3 amino acids, less than about 2 amino acids, or less than about 1 amino acids.

In a particular (but non-limiting) embodiment, and alternatively and/or in addition to the above embodiments, the antibody or functional fragment thereof has a light chain variable region with an amino acid sequence that is at least about 70% identical to any of the light chain variable region amino acid sequences outlined in Tables 2-14 (i.e., SEQ ID NO:18, 38, 58, 78, 98, 118, 138, 158, 178, 198, 218, 238, or 258), such as (but not limited to) at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO:18, 38, 58, 78, 98, 118, 138, 158, 178, 198, 218, 238, or 258.

In a particular (but non-limiting) embodiment, the antibody or functional fragment thereof has a light chain variable region with an amino acid sequence that differs from SEQ ID NO:18, 38, 58, 78, 98, 118, 138, 158, 178, 198, 218, 238, or 258 by less than about 21 amino acids, less than about 20 amino acids, less than about 19 amino acids, less than about 18 amino acids, less than about 17 amino acids, less than about 16 amino acids, less than about 15 amino acids, less than about 14 amino acids, less than about 13 amino acids, less than about 12 amino acids, less than about 11 amino acids, less than about 10 amino acids, less than about 9 amino acids, less than about 8 amino acids, less than about 7 amino acids, less than about 6 amino acids, less than about 5 amino acids, less than about 4 amino acids, less than about 3 amino acids, less than about 2 amino acids, or less than about 1 amino acids.

In a particular (but non-limiting) embodiment, the antibody or functional fragment thereof has a heavy chain variable region with an amino acid sequence that is at least about 90% identical to SEQ ID NO:17, 37, 57, 77, 97, 117, 137, 157, 177, 197, 217, 237, or 257, and/or the antibody or functional fragment thereof has a light chain variable region with an amino acid sequence that is at least about 90% identical to SEQ ID NO:18, 38, 58, 78, 98, 118, 138, 158, 178, 198, 218, 238, or 258. In a particular (but non-limiting) embodiment, the antibody or functional fragment thereof has a heavy chain variable region with an amino acid sequence that differs from SEQ ID NO:17, 37, 57, 77, 97, 117, 137, 157, 177, 197, 217, 237, or 257 by less than about 12 amino acids, and/or a light chain variable region with an amino acid sequence that differs from SEQ ID NO:18, 38, 58, 78, 98, 118, 138, 158, 178, 198, 218, 238, or 258 by less than about 12 amino acids.

In another particular (but non-limiting) embodiment, the antibody or functional fragment thereof has a heavy chain variable region with the amino acid sequence of SEQ ID NO:17, 37, 57, 77, 97, 117, 137, 157, 177, 197, 217, 237, or 257 and/or a light chain variable region with the amino acid sequence of SEQ ID NO:18, 38, 58, 78, 98, 118, 138, 158, 178, 198, 218, 238, or 258.

In another particular (but non-limiting) embodiment, the antibody or functional fragment thereof has a heavy chain with an amino acid sequence that is at least about 70% identical to any of the heavy chain amino acid sequences outlined in Tables 2-14 (i.e., SEQ ID NO:1, 21, 41, 61, 81, 101, 121, 141, 161, 181, 201, 221, or 241), such as (but not limited to) at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO:1, 21, 41, 61, 81, 101, 121, 141, 161, 181, 201, 221, or 241.

In a particular (but non-limiting) embodiment, the antibody or functional fragment thereof has a heavy chain with an amino acid sequence that differs from SEQ ID NO:1, 21, 41, 61, 81, 101, 121, 141, 161, 181, 201, 221, or 241 by less than about 100 amino acids, less than about 90 amino acids, less than about 80 amino acids, less than about 75 amino acids, less than about 70 amino acids, less than about 65 amino acids, less than about 60 amino acids, less than about 55 amino acids, less than about 50 amino acids, less than about 45 amino acids, less than about 40 amino acids, less than about 35 amino acids, less than about 30 amino acids, less than about 25 amino acids, less than about 24 amino acids, less than about 23 amino acids, less than about 22 amino acids, less than about 21 amino acids, less than about 20 amino acids, less than about 19 amino acids, less than about 18 amino acids, less than about 17 amino acids, less than about 16 amino acids, less than about 15 amino acids, less than about 14 amino acids, less than about 13 amino acids, less than about 12 amino acids, less than about 11 amino acids, less than about 10 amino acids, less than about 9 amino acids, less than about 8 amino acids, less than about 7 amino acids, less than about 6 amino acids, less than about 5 amino acids, less than about 4 amino acids, less than about 3 amino acids, less than about 2 amino acids, or less than about 1 amino acids.

In a particular (but non-limiting) embodiment, and alternatively and/or in addition to the above embodiments, the antibody or functional fragment thereof has a light chain with an amino acid sequence that is at least about 70% identical to any of the light chain amino acid sequences outlined in Tables 2-14 (i.e., SEQ ID NO:5, 25, 45, 65, 85, 105, 125, 145, 165, 185, 205, 225, or 245), such as (but not limited to) at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO:5, 25, 45, 65, 85, 105, 125, 145, 165, 185, 205, 225, or 245.

In a particular (but non-limiting) embodiment, the antibody or functional fragment thereof has a light chain with an amino acid sequence that differs from SEQ ID NO:5, 25, 45, 65, 85, 105, 125, 145, 165, 185, 205, 225, or 245 by less than about 45 amino acids, less than about 40 amino acids, less than about 35 amino acids, less than about 30 amino acids, less than about 25 amino acids, less than about 24 amino acids, less than about 23 amino acids, less than about 22 amino acids, less than about 21 amino acids, less than about 20 amino acids, less than about 19 amino acids, less than about 18 amino acids, less than about 17 amino acids, less than about 16 amino acids, less than about 15 amino acids, less than about 14 amino acids, less than about 13 amino acids, less than about 12 amino acids, less than about 11 amino acids, less than about 10 amino acids, less than about 9 amino acids, less than about 8 amino acids, less than about 7 amino acids, less than about 6 amino acids, less than about 5 amino acids, less than about 4 amino acids, less than about 3 amino acids, less than about 2 amino acids, or less than about 1 amino acids.

In a particular (but non-limiting) embodiment, the antibody or functional fragment thereof has a heavy chain with an amino acid sequence that is at least about 90% identical to SEQ ID NO:1, 21, 41, 61, 81, 101, 121, 141, 161, 181, 201, 221, or 241, and/or the antibody or functional fragment thereof has a light chain with an amino acid sequence that is at least about 70% identical to SEQ ID NO:5, 25, 45, 65, 85, 105, 125, 145, 165, 185, 205, 225, or 245. In a particular (but non-limiting) embodiment, the antibody or functional fragment thereof has a heavy chain with an amino acid sequence that differs from SEQ ID NO:1, 21, 41, 61, 81, 101, 121, 141, 161, 181, 201, 221, or 241 by less than about 46 amino acids, and/or a light chain with an amino acids sequence that differs from SEQ ID NO:5, 25, 45, 65, 85, 105, 125, 145, 165, 185, 205, 225, or 245 by less than about 24 amino acids.

In another particular (but non-limiting) embodiment, the antibody or functional fragment thereof has a heavy chain with the amino acid sequence of SEQ ID NO:1, 21, 41, 61, 81, 101, 121, 141, 161, 181, 201, 221, or 241 and/or a light chain with the amino acid sequence of SEQ ID NO:5, 25, 45, 65, 85, 105, 125, 145, 165, 185, 205, 225, or 245.

In yet another particular (but non-limiting) embodiment, the antibody or functional fragment has a heavy chain encoded by a polynucleotide that is at least about 70% identical to SEQ ID NO:9, 29, 49, 69, 89, 109, 129, 149, 169, 189, 209, 229, or 249 (and/or a heavy chain variable region encoded by a polynucleotide that is at least about 70% identical to SEQ ID NO:19, 39, 59, 79, 99, 119, 139, 159, 179, 199, 219, 239, or 259), such as (but not limited to) at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO:9, 29, 49, 69, 89, 109, 129, 149, 169, 189, 209, 229, or 249 and/or SEQ ID NO:19, 39, 59, 79, 99, 119, 139, 159, 179, 199, 219, 239, or 259.

In yet another particular (but non-limiting) embodiment, and alternatively and/or in addition to the above embodiment, the antibody or functional fragment has a light chain encoded by a polynucleotide that is at least about 70% identical to SEQ ID NO:13, 33, 53, 73, 93, 113, 133, 153, 173, 193, 213, 233, or 253 (and/or a light chain variable region encoded by a polynucleotide that is at least about 70% identical to SEQ ID NO:20, 40, 60, 80, 100, 120, 140, 160, 180, 200, 220, 240, or 260), such as (but not limited to) at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO:13, 33, 53, 73, 93, 113, 133, 153, 173, 193, 213, 233, or 253 and/or SEQ ID NO:20, 40, 60, 80, 100, 120, 140, 160, 180, 200, 220, 240, or 260.

In yet a further particular (but non-limiting) embodiment, the antibody or functional fragment has a heavy chain encoded by a polynucleotide that is at least about 70% identical to SEQ ID NO:9, 29, 49, 69, 89, 109, 129, 149, 169, 189, 209, 229, or 249, and/or the antibody or functional fragment has a light chain encoded by a polynucleotide that is at least about 70% identical to SEQ ID NO:13, 33, 53, 73, 93, 113, 133, 153, 173, 193, 213, 233, or 253.

In yet a further particular (but non-limiting) embodiment, the antibody or functional fragment has a heavy chain variable region encoded by a polynucleotide that is at least about 70% identical to SEQ ID NO:19, 39, 59, 79, 99, 119, 139, 159, 179, 199, 219, 239, or 259, and/or the antibody or functional fragment has a light chain variable region encoded by a polynucleotide that is at least about 70% identical to SEQ ID NO:20, 40, 60, 80, 100, 120, 140, 160, 180, 200, 220, 240, or 260.

In yet another particular (but non-limiting) embodiment, the antibody or functional fragment thereof has a heavy chain encoded by a sequence that differs from SEQ ID NO:9, 29, 49, 69, 89, 109, 129, 149, 169, 189, 209, 229, or 249 (and/or a heavy chain variable region encoded by a sequence that differs from SEQ ID NO:19, 39, 59, 79, 99, 119, 139, 159, 179, 199, 219, 239, or 259) by less than about 100 nucleotides, less than about 90 nucleotides, less than about 80 nucleotides, less than about 75 nucleotides, less than about 70 nucleotides, less than about 60 nucleotides, less than about 50 nucleotides, less than about 45 nucleotides, less than about 40 nucleotides, less than about 35 nucleotides, less than about 30 nucleotides, less than about 25 nucleotides, less than about 20 nucleotides, less than about 15 nucleotides, less than about 10 nucleotides, less than about 9 nucleotides, less than about 8 nucleotides, less than about 7 nucleotides, less than about 6 nucleotides, less than about 5 nucleotides, less than about 4 nucleotides, less than about 3 nucleotides, less than about 2 nucleotides, or less than about 1 nucleotide.

In yet another particular (but non-limiting) embodiment, and alternatively and/or in addition to the above embodiment, the antibody or functional fragment thereof has a light chain encoded by a sequence that differs from SEQ ID NO:9, 29, 49, 69, 89, 109, 129, 149, 169, 189, 209, 229, or 249 (and/or a light chain variable region encoded by a sequence that differs from SEQ ID NO:20, 40, 60, 80, 100, 120, 140, 160, 180, 200, 220, 240, or 260) by less than about 100 nucleotides, less than about 90 nucleotides, less than about 80 nucleotides, less than about 75 nucleotides, less than about 70 nucleotides, less than about 60 nucleotides, less than about 50 nucleotides, less than about 45 nucleotides, less than about 40 nucleotides, less than about 35 nucleotides, less than about 30 nucleotides, less than about 25 nucleotides, less than about 20 nucleotides, less than about 15 nucleotides, less than about 10 nucleotides, less than about 9 nucleotides, less than about 8 nucleotides, less than about 7 nucleotides, less than about 6 nucleotides, less than about 5 nucleotides, less than about 4 nucleotides, less than about 3 nucleotides, less than about 2 nucleotides, or less than about 1 nucleotide.

In yet another particular (but non-limiting) embodiment, the antibody or functional fragment thereof has a heavy chain encoded by a sequence that differs from SEQ ID NO:9, 29, 49, 69, 89, 109, 129, 149, 169, 189, 209, 229, or 249 by less than about 100 nucleotides and/or a light chain encoded by a sequence that differs from SEQ ID NO:13, 33, 53, 73, 93, 113, 133, 153, 173, 193, 213, 233, or 253 by less than about 70 nucleotides.

In yet another particular (but non-limiting) embodiment, the antibody or functional fragment thereof has a heavy chain variable region encoded by a sequence that differs from SEQ ID NO:19, 39, 59, 79, 99, 119, 139, 159, 179, 199, 219, 239, or 259 by less than about 35 nucleotides and/or a light chain variable region encoded by a sequence that differs from SEQ ID NO:20, 40, 60, 80, 100, 120, 140, 160, 180, 200, 220, 240, or 260 by less than about 34 nucleotides.

Certain non-limiting embodiments of the present disclosure are directed to an antibody or functional fragment thereof that specifically binds to naltrexone (and in certain particular (but non-limiting) embodiments, the conjugate of Formula III, wherein R₂ is a cyclopropyl), wherein the antibody comprises one or more of: (i) a heavy chain variable region CDR1 encoded by a sequence of SEQ ID NO:10; (ii) a heavy chain variable region CDR2 encoded by a sequence of SEQ ID NO:11; (iii) a heavy chain variable region CDR3 encoded by a sequence of SEQ ID NO:12; (iv) a light chain variable region CDR1 encoded by a sequence of SEQ ID NO:14; (v) a light chain variable region CDR2 encoded by a sequence of SEQ ID NO:15 ;and (vi) a light chain variable region CDR3 encoded by a sequence of SEQ ID NO:16. In a particular non-limiting embodiment, the antibody or functional fragment thereof comprises two, three, four, or five of (i)-(vi) above. In another particular non-limiting embodiment, the antibody or functional fragment thereof comprises all of (i)-(vi) above.

Certain non-limiting embodiments of the present disclosure are directed to an antibody or functional fragment thereof that specifically binds to naltrexone (and in certain particular (but non-limiting) embodiments, the conjugate of Formula III, wherein R₂ is a cyclopropyl), wherein the antibody comprises one or more of: (i) a heavy chain variable region CDR1 encoded by a sequence of SEQ ID NO:30; (ii) a heavy chain variable region CDR2 encoded by a sequence of SEQ ID NO:31; (iii) a heavy chain variable region CDR3 encoded by a sequence of SEQ ID NO:32; (iv) a light chain variable region CDR1 encoded by a sequence of SEQ ID NO:34; (v) a light chain variable region CDR2 encoded by a sequence of SEQ ID NO:35; and (vi) a light chain variable region CDR3 encoded by a sequence of SEQ ID NO:36. In a particular non-limiting embodiment, the antibody or functional fragment thereof comprises two, three, four, or five of (i)-(vi) above. In another particular non-limiting embodiment, the antibody or functional fragment thereof comprises all of (i)-(vi) above.

Certain non-limiting embodiments of the present disclosure are directed to an antibody or functional fragment thereof that specifically binds to naltrexone (and in certain particular (but non-limiting) embodiments, the conjugate of Formula III, wherein R₂ is a cyclopropyl), wherein the antibody comprises one or more of: (i) a heavy chain variable region CDR1 encoded by a sequence of SEQ ID NO:50; (ii) a heavy chain variable region CDR2 encoded by a sequence of SEQ ID NO:51; (iii) a heavy chain variable region CDR3 encoded by a sequence of SEQ ID NO:52; (iv) a light chain variable region CDR1 encoded by a sequence of SEQ ID NO:54; (v) a light chain variable region CDR2 encoded by a sequence of SEQ ID NO:55; and (vi) a light chain variable region CDR3 encoded by a sequence of SEQ ID NO:56. In a particular non-limiting embodiment, the antibody or functional fragment thereof comprises two, three, four, or five of (i)-(vi) above. In another particular non-limiting embodiment, the antibody or functional fragment thereof comprises all of (i)-(vi) above.

Certain non-limiting embodiments of the present disclosure are directed to an antibody or functional fragment thereof that specifically binds to naltrexone (and in certain particular (but non-limiting) embodiments, the conjugate of Formula III, wherein R₂ is a cyclopropyl), wherein the antibody comprises one or more of: (i) a heavy chain variable region CDR1 encoded by a sequence of SEQ ID NO:70; (ii) a heavy chain variable region CDR2 encoded by a sequence of SEQ ID NO:71; (iii) a heavy chain variable region CDR3 encoded by a sequence of SEQ ID NO:72; (iv) a light chain variable region CDR1 encoded by a sequence of SEQ ID NO:74; (v) a light chain variable region CDR2 encoded by a sequence of SEQ ID NO:75; and (vi) a light chain variable region CDR3 encoded by a sequence of SEQ ID NO:76. In a particular non-limiting embodiment, the antibody or functional fragment thereof comprises two, three, four, or five of (i)-(vi) above. In another particular non-limiting embodiment, the antibody or functional fragment thereof comprises all of (i)-(vi) above.

Certain non-limiting embodiments of the present disclosure are directed to an antibody or functional fragment thereof that specifically binds to naltrexone (and in certain particular (but non-limiting) embodiments, the conjugate of Formula III, wherein R₂ is a cyclopropyl), wherein the antibody comprises one or more of: (i) a heavy chain variable region CDR1 encoded by a sequence of SEQ ID NO:90; (ii) a heavy chain variable region CDR2encoded by a sequence of SEQ ID NO:91; (iii) a heavy chain variable region CDR3 encoded by a sequence of SEQ ID NO:92; (iv) a light chain variable region CDR1 encoded by a sequence of SEQ ID NO:94; (v) a light chain variable region CDR2 encoded by a sequence of SEQ ID NO:95; and (vi) a light chain variable region CDR3 encoded by a sequence of SEQ ID NO:96. In a particular non-limiting embodiment, the antibody or functional fragment thereof comprises two, three, four, or five of (i)-(vi) above. In another particular non-limiting embodiment, the antibody or functional fragment thereof comprises all of (i)-(vi) above.

Certain non-limiting embodiments of the present disclosure are directed to an antibody or functional fragment thereof that specifically binds to naltrexone (and in certain particular (but non-limiting) embodiments, the conjugate of Formula III, wherein R₂ is a cyclopropyl), wherein the antibody comprises one or more of: (i) a heavy chain variable region CDR1 encoded by a sequence of SEQ ID NO:110; (ii) a heavy chain variable region CDR2 encoded by a sequence of SEQ ID NO:111; (iii) a heavy chain variable region CDR3 encoded by a sequence of SEQ ID NO:112; (iv) a light chain variable region CDR1 encoded by a sequence of SEQ ID NO:114; (v) a light chain variable region CDR2 encoded by a sequence of SEQ ID NO:115; and (vi) a light chain variable region CDR3 encoded by a sequence of SEQ ID NO:116. In a particular non-limiting embodiment, the antibody or functional fragment thereof comprises two, three, four, or five of (i)-(vi) above. In another particular non-limiting embodiment, the antibody or functional fragment thereof comprises all of (i)-(vi) above.

Certain non-limiting embodiments of the present disclosure are directed to an antibody or functional fragment thereof that specifically binds to naltrexone (and in certain particular (but non-limiting) embodiments, the conjugate of Formula III, wherein R₂ is a cyclopropyl), wherein the antibody comprises one or more of: (i) a heavy chain variable region CDR1 encoded by a sequence of SEQ ID NO:130; (ii) a heavy chain variable region CDR2 encoded by a sequence of SEQ ID NO:131; (iii) a heavy chain variable region CDR3 encoded by a sequence of SEQ ID NO:132; (iv) a light chain variable region CDR1 encoded by a sequence of SEQ ID NO:134; (v) a light chain variable region CDR2 encoded by a sequence of SEQ ID NO:135; and (vi) a light chain variable region CDR3 encoded by a sequence of SEQ ID NO:136. In a particular non-limiting embodiment, the antibody or functional fragment thereof comprises two, three, four, or five of (i)-(vi) above. In another particular non-limiting embodiment, the antibody or functional fragment thereof comprises all of (i)-(vi) above.

Certain non-limiting embodiments of the present disclosure are directed to an antibody or functional fragment thereof that specifically binds to naltrexone (and in certain particular (but non-limiting) embodiments, the conjugate of Formula III, wherein R₂ is a cyclopropyl), wherein the antibody comprises one or more of: (i) a heavy chain variable region CDR1 encoded by a sequence of SEQ ID NO:150; (ii) a heavy chain variable region CDR2 encoded by a sequence of SEQ ID NO:151; (iii) a heavy chain variable region CDR3 encoded by a sequence of SEQ ID NO:152; (iv) a light chain variable region CDR1 encoded by a sequence of SEQ ID NO:154; (v) a light chain variable region CDR2 encoded by a sequence of SEQ ID NO:155; and (vi) a light chain variable region CDR3 encoded by a sequence of SEQ ID NO:156. In a particular non-limiting embodiment, the antibody or functional fragment thereof comprises two, three, four, or five of (i)-(vi) above. In another particular non-limiting embodiment, the antibody or functional fragment thereof comprises all of (i)-(vi) above.

Certain non-limiting embodiments of the present disclosure are directed to an antibody or functional fragment thereof that specifically binds to naloxone (and in certain particular (but non-limiting) embodiments, the conjugate of Formula III, wherein R₂ is a cyclopropyl), wherein the antibody comprises one or more of: (i) a heavy chain variable region CDR1 encoded by a sequence of SEQ ID NO:170; (ii) a heavy chain variable region CDR2encoded by a sequence of SEQ ID NO:171; (iii) a heavy chain variable region CDR3 encoded by a sequence of SEQ ID NO:172; (iv) a light chain variable region CDR1 encoded by a sequence of SEQ ID NO:174; (v) a light chain variable region CDR2 encoded by a sequence of SEQ ID NO:175; and (vi) a light chain variable region CDR3 encoded by a sequence of SEQ ID NO:176. In a particular non-limiting embodiment, the antibody or functional fragment thereof comprises two, three, four, or five of (i)-(vi) above. In another particular non-limiting embodiment, the antibody or functional fragment thereof comprises all of (i)-(vi) above.

Certain non-limiting embodiments of the present disclosure are directed to an antibody or functional fragment thereof that specifically binds to naloxone (and in certain particular (but non-limiting) embodiments, the conjugate of Formula III, wherein R₂ is a cyclopropyl), wherein the antibody comprises one or more of: (i) a heavy chain variable region CDR1 encoded by a sequence of SEQ ID NO:190; (ii) a heavy chain variable region CDR2 encoded by a sequence of SEQ ID NO:191; (iii) a heavy chain variable region CDR3 encoded by a sequence of SEQ ID NO:192; (iv) a light chain variable region CDR1 encoded by a sequence of SEQ ID NO:194; (v) a light chain variable region CDR2 encoded by a sequence of SEQ ID NO:195; and (vi) a light chain variable region CDR3 encoded by a sequence of SEQ ID NO:196. In a particular non-limiting embodiment, the antibody or functional fragment thereof comprises two, three, four, or five of (i)-(vi) above. In another particular non-limiting embodiment, the antibody or functional fragment thereof comprises all of (i)-(vi) above.

Certain non-limiting embodiments of the present disclosure are directed to an antibody or functional fragment thereof that specifically binds to naloxone (and in certain particular (but non-limiting) embodiments, the conjugate of Formula III, wherein R₂ is a cyclopropyl), wherein the antibody comprises one or more of: (i) a heavy chain variable region CDR1 encoded by a sequence of SEQ ID NO:210; (ii) a heavy chain variable region CDR2 encoded by a sequence of SEQ ID NO:211; (iii) a heavy chain variable region CDR3 encoded by a sequence of SEQ ID NO:212; (iv) a light chain variable region CDR1 encoded by a sequence of SEQ ID NO:214; (v) a light chain variable region CDR2 encoded by a sequence of SEQ ID NO:215; and (vi) a light chain variable region CDR3 encoded by a sequence of SEQ ID NO:216. In a particular non-limiting embodiment, the antibody or functional fragment thereof comprises two, three, four, or five of (i)-(vi) above. In another particular non-limiting embodiment, the antibody or functional fragment thereof comprises all of (i)-(vi) above.

Certain non-limiting embodiments of the present disclosure are directed to an antibody or functional fragment thereof that specifically binds to naloxone (and in certain particular (but non-limiting) embodiments, the conjugate of Formula III, wherein R₂ is a cyclopropyl), wherein the antibody comprises one or more of: (i) a heavy chain variable region CDR1 encoded by a sequence of SEQ ID NO:230; (ii) a heavy chain variable region CDR2 encoded by a sequence of SEQ ID NO:231; (iii) a heavy chain variable region CDR3 encoded by a sequence of SEQ ID NO:232; (iv) a light chain variable region CDR1 encoded by a sequence of SEQ ID NO:234; (v) a light chain variable region CDR2 encoded by a sequence of SEQ ID NO:235; and (vi) a light chain variable region CDR3 encoded by a sequence of SEQ ID NO:236. In a particular non-limiting embodiment, the antibody or functional fragment thereof comprises two, three, four, or five of (i)-(vi) above. In another particular non-limiting embodiment, the antibody or functional fragment thereof comprises all of (i)-(vi) above.

Certain non-limiting embodiments of the present disclosure are directed to an antibody or functional fragment thereof that specifically binds to naloxone (and in certain particular (but non-limiting) embodiments, the conjugate of Formula III, wherein R₂ is a cyclopropyl), wherein the antibody comprises one or more of: (i) a heavy chain variable region CDR1 encoded by a sequence of SEQ ID NO:250; (ii) a heavy chain variable region CDR2encoded by a sequence of SEQ ID NO:251; (iii) a heavy chain variable region CDR3 encoded by a sequence of SEQ ID NO:252; (iv) a light chain variable region CDR1 encoded by a sequence of SEQ ID NO:254; (v) a light chain variable region CDR2 encoded by a sequence of SEQ ID NO:255; and (vi) a light chain variable region CDR3 encoded by a sequence of SEQ ID NO:256. In a particular non-limiting embodiment, the antibody or functional fragment thereof comprises two, three, four, or five of (i)-(vi) above. In another particular non-limiting embodiment, the antibody or functional fragment thereof comprises all of (i)-(vi) above.

The antibody or functional fragment thereof may be a monoclonal antibody or functional fragment thereof. Alternatively, the antibody or functional fragment thereof may be a polyclonal antibody or functional fragment thereof.

In certain non-limiting embodiments, the antibody or functional fragment thereof is further defined as being selected from a full length immunoglobulin molecule, an scFv, a Fab fragment, a Fab′ fragment, a F(ab′)2, a Fv, a disulfide linked Fv, and combinations thereof.

In certain non-limiting embodiments, the antibody or functional fragment thereof is isolated. In particular (but non-limiting) embodiments, the antibody or functional fragment thereof is purified.

The present disclosure is also directed to antibodies or functional fragments thereof that bind to the same epitope as any of the antibodies or functional fragments described herein above.

Certain non-limiting embodiments of the present disclosure are also directed to a method of producing an antibody or functional fragment thereof which can specifically bind to naloxone and/or naltrexone. The method includes immunizing a non-human animal with an antigenic compound comprising a conjugate of a carrier protein attached to naloxone and/or naltrexone; and recovering the antibody or functional fragment thereof from blood plasma of the non-human animal.

Certain non-limiting embodiments of the present disclosure are also directed to a method of producing an antibody or functional fragment thereof which can specifically bind to naloxone and/or naltrexone. The method includes immunizing a non-human animal with an antigenic compound comprising a conjugate of a carrier protein attached to naloxone and/or naltrexone, thereby inducing B-cells producing antibodies that bind to the conjugate; and obtaining an antibody or functional fragment thereof produced by the B-cells. The antibody or functional fragment thereof can be obtained by various means known in the art, such as (but not limited to) via hybridoma technology or by B-cell PCR technology. The method may further include the optional step of further selecting for the antibody or functional fragment thereof based on binding to naloxone and/or naltrexone.

Certain non-limiting embodiments of the present disclosure are directed to a hybridoma producing any of the antibodies or functional fragments thereof described herein above.

Certain non-limiting embodiments of the present disclosure are directed to a method of producing an antibody or functional fragment thereof which can specifically bind to naloxone and/or naltrexone. In the method, a hybridoma capable of producing any of the antibodies or functional fragments thereof described or otherwise contemplated herein is cultured to produce any of the antibodies or functional fragments thereof described herein above. In at least certain non-limiting embodiments, the antibody or functional fragment thereof is recovered.

Certain non-limiting embodiments of the present disclosure are also directed to a conjugate comprising any of the antibodies or functional fragments thereof disclosed or otherwise contemplated herein attached to a detectable label. Non-limiting examples of detectable labels that may be utilized in accordance with the present disclosure include an enzyme label, a radioactive label, a fluorescent label, a chemiluminescent label, a bioluminescent label, and a particulate label, as well as any combinations thereof. In addition, the detectable label may be attached to the antibody or functional fragment via direct or indirect conjugation.

Certain non-limiting embodiments of the present disclosure are also directed to a conjugate comprising any of the antibodies or functional fragments thereof disclosed or otherwise contemplated herein attached to a solid support. Attachment of the antibody/function fragment to a solid support (via direct or indirect conjugation) produces an affinity purification chromatography substrate such as, for example, a column.

Certain non-limiting embodiments of the present disclosure are directed to a polynucleotide encoding any of the antibodies or functional fragments thereof disclosed or otherwise contemplated herein.

In a particular (but non-limiting) embodiment, a portion of the polynucleotide encoding a heavy chain of the antibody or functional fragment thereof is at least about 70% identical to SEQ ID NO:9, 29, 49, 69, 89, 109, 129, 149, 169, 189, 209, 229, or 249 (and/or a portion of the polynucleotide encoding a heavy chain variable region of the antibody or functional fragment thereof is at least about 70% identical to SEQ ID NO:19, 39, 59, 79, 99, 119, 139, 159, 179, 199, 219, 239, or 259), such as (but not limited to) at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO:9, 29, 49, 69, 89, 109, 129, 149, 169, 189, 209, 229, or 249 and/or SEQ ID NO:19, 39, 59, 79, 99, 119, 139, 159, 179, 199, 219, 239, or 259.

In yet another particular (but non-limiting) embodiment, and alternatively and/or in addition to the above embodiment, a portion of the polynucleotide encoding a light chain of the antibody or functional fragment thereof is at least about 70% identical to SEQ ID NO:13, 33, 53, 73, 93, 113, 133, 153, 173, 193, 213, 233, or 253 (and/or a portion of the polynucleotide encoding a light chain variable region of the antibody or functional fragment thereof is at least about 70% identical to SEQ ID NO:20, 40, 60, 80, 100, 120, 140, 160, 180, 200, 220, 240, or 260), such as (but not limited to) at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical, or at least about 99% identical to SEQ ID NO:13, 33, 53, 73, 93, 113, 133, 153, 173, 193, 213, 233, or 253 and/or SEQ ID NO:20, 40, 60, 80, 100, 120, 140, 160, 180, 200, 220, 240, or 260.

In yet another particular (but non-limiting) embodiment, a portion of the polynucleotide encoding a heavy chain of the antibody or functional fragment thereof is at least about 90% identical to SEQ ID NO:9, 29, 49, 69, 89, 109, 129, 149, 169, 189, 209, 229, or 249 and/or a portion of the polynucleotide encoding a light chain of the antibody or functional fragment thereof is at least about 90% identical to SEQ ID NO:13, 33, 53, 73, 93, 113, 133, 153, 173, 193, 213, 233, or 253.

In yet another particular (but non-limiting) embodiment, a portion of the polynucleotide encoding a heavy chain variable region of the antibody or functional fragment thereof is at least about 90% identical to SEQ ID NO:19, 39, 59, 79, 99, 119, 139, 159, 179, 199, 219, 239, or 259 and/or a portion of the polynucleotide encoding a light chain variable region of the antibody or functional fragment thereof is at least about 90% identical to SEQ ID NO:20, 40, 60, 80, 100, 120, 140, 160, 180, 200, 220, 240, or 260.

In another particular (but non-limiting) embodiment, the portion of the sequence corresponding to the heavy chain of the antibody or functional fragment thereof and/or the portion of the sequence corresponding to the light chain of the antibody or functional fragment thereof differs from SEQ ID NO:9, 29, 49, 69, 89, 109, 129, 149, 169, 189, 209, 229, or 249 or SEQ ID NO:13, 33, 53, 73, 93, 113, 133, 153, 173, 193, 213, 233, or 253, respectively (and/or the portion of the sequence corresponding to the heavy chain variable region of the antibody or functional fragment thereof and/or the portion of the sequence corresponding to the light chain variable region of the antibody or functional fragment thereof differs from SEQ ID NO:19, 39, 59, 79, 99, 119, 139, 159, 179, 199, 219, 239, or 259 or SEQ ID NO:20, 40, 60, 80, 100, 120, 140, 160, 180, 200, 220, 240, or 260, respectively), by less than about 100 nucleotides, less than about 90 nucleotides, less than about 80 nucleotides, less than about 75 nucleotides, less than about 70 nucleotides, less than about 60 nucleotides, less than about 50 nucleotides, less than about 45 nucleotides, less than about 40 nucleotides, less than about 35 nucleotides, less than about 30 nucleotides, less than about 25 nucleotides, less than about 20 nucleotides, less than about 15 nucleotides, less than about 10 nucleotides, less than about 9 nucleotides, less than about 8 nucleotides, less than about 7 nucleotides, less than about 6 nucleotides, less than about 5 nucleotides, less than about 4 nucleotides, less than about 3 nucleotides, less than about 2 nucleotides, or less than about 1 nucleotide.

In yet another particular (but non-limiting) embodiment, the portion of the sequence corresponding to the heavy chain of the antibody or functional fragment thereof differs from SEQ ID NO:9, 29, 49, 69, 89, 109, 129, 149, 169, 189, 209, 229, or 249 by less than about 100 nucleotides, and/or the portion of the sequence corresponding to the light chain of the antibody or functional fragment thereof differs from SEQ ID NO:13, 33, 53, 73, 93, 113, 133, 153, 173, 193, 213, 233, or 253 by less than about 70 nucleotides.

In yet another particular (but non-limiting) embodiment, the portion of the sequence corresponding to the heavy chain variable region of the antibody or functional fragment thereof differs from SEQ ID NO:19, 39, 59, 79, 99, 119, 139, 159, 179, 199, 219, 239, or 259 by less than about 35 nucleotides, and/or the portion of the sequence corresponding to the light chain variable region of the antibody or functional fragment thereof differs from SEQ ID NO:20, 40, 60, 80, 100, 120, 140, 160, 180, 200, 220, 240, or 260 by less than about 34 nucleotides.

Certain non-limiting embodiments of the present disclosure are directed to a vector comprising any of the polynucleotides encoding an antibody or functional fragment thereof described or otherwise contemplated herein.

Certain non-limiting embodiments of the present disclosure are directed to a recombinant host cell comprising any of the polynucleotides encoding an antibody or functional fragment thereof described or otherwise contemplated herein. Certain non-limiting embodiments of the present disclosure are directed to a recombinant host cell comprising any of the vectors described or otherwise contemplated herein.

Certain non-limiting embodiments of the present disclosure are directed to a method of producing an antibody or functional fragment thereof which can specifically bind to naloxone and/or naltrexone. The method comprises: (a) culturing any of the recombinant host cells described or otherwise contemplated herein in a cell culture under conditions that allow for the expression of the antibody or functional fragment thereof encoded by the polynucleotide; and (b) isolating the antibody or functional fragment thereof from the cell culture.

Certain non-limiting embodiments of the present disclosure are directed to a method of detecting naloxone and/or naltrexone present in a biological sample. The method comprises: contacting the biological sample with any of the antibodies or functional fragments thereof disclosed or otherwise contemplated herein under conditions whereby an antibody/naloxone and/or antibody/naltrexone complex is formed if naloxone and/or naltrexone, respectively, is present in the sample; and detecting any antibody/naloxone and/or antibody/naltrexone complex formed, wherein an amount of antibody/naloxone and/or antibody/naltrexone complex formed is directly proportional to an amount of naloxone and/or naltrexone, respectively, present in the sample.

In a particular (but non-limiting) embodiment, a label as described herein above is attached to the antibody/functional fragment for use in the detection of the antibody/naloxone and/or antibody/naltrexone complex.

Certain non-limiting embodiments of the present disclosure are directed to methods of substantially reducing (or substantially removing) naloxone and/or naltrexone interference from an opiate assay of a biological sample. The method comprises contacting the biological sample with one or more of any of the antibodies or functional fragments thereof disclosed or otherwise contemplated herein under conditions whereby an antibody/naloxone and/or antibody/naltrexone complex is formed if naloxone and/or naltrexone, respectively, is present in the sample; removing any antibody/naloxone and/or antibody/naltrexone complex formed; and performing the opiate assay on the biological sample in which naloxone and/or naltrexone has been substantially reduced/removed. This method may be utilized with an assay for any opiate as well as any opiate assay format. For example (but not by way of limitation), the opiate to be assayed may be codeine, hydrocodone, hydromorphone, morphine, oxycodone, oxymorphone, heroin (6-acetylmorphine), combinations thereof, and the like. Non-limiting examples of opiate assay formats include ELISAs, chip assays, LC/MS/MS, immunoassays, enzyme immunoassays, enzyme multiplied immunoassays, fluorescence polarization immunoassays, combinations thereof, and the like. Various types of opiate assays are well known and well used in the art. Thus, no further description thereof is deemed necessary to allow a person of ordinary skill in the art to perform the methods disclosed herein.

In a particular (but non-limiting) embodiment, the at least one antibody or functional fragment thereof used in the method of substantially reducing (or substantially removing) naloxone and/or naltrexone interference from an opiate assay includes at least two antibodies or functional fragments thereof: (1) at least one antibody or functional fragment thereof that specifically binds naloxone, and (2) at least one antibody or functional fragment thereof that specifically binds naltrexone.

Certain non-limiting embodiments of the present disclosure also include kits containing any of the antibodies/functional fragments and/or compositions disclosed or otherwise contemplated herein (such as the compositions comprising antibody/functional fragment attached to a detectable label or a solid support), as well as any other reagents that may be utilized in the assays/methods described herein. For example (but not by way of limitation), the kit may further include one or more components of the opiate assays described herein above. For example (but not by way of limitation), the kit may include an antibody or functional fragment thereof that binds to any of the opiates described or otherwise contemplated herein, for use in an opiate immunoassay.

The assay components/reagents of the compositions/kits/methods may be provided in any form that allows them to function in accordance with the present disclosure. For example, but not by way of limitation, each of the reagents may be provided in liquid form and disposed in bulk and/or single aliquot form within the kit. Alternatively, in a particular (but non-limiting) embodiment, one or more of the reagents may be disposed in the kit in the form of a single aliquot lyophilized reagent. The use of dried reagents in microfluidics devices is described in detail in U.S. Pat. No. 9,244,085 (Samproni), the entire contents of which are hereby expressly incorporated herein by reference.

In addition to the assay components/reagents described in detail herein above, the kits may further contain other reagent(s) for conducting any of the particular assays described or otherwise contemplated herein. The nature of these additional reagent(s) will depend upon the particular assay format, and identification thereof is well within the skill of one of ordinary skill in the art; therefore, no further description thereof is deemed necessary. Also, the components/reagents present in the kits may each be in separate containers/compartments, or various components/reagents can be combined in one or more containers/compartments, depending on the cross-reactivity and stability of the components/reagents. In addition, the kit may include a microfluidics device in which the components/reagents are disposed.

The relative amounts of the various components/reagents in the kits can vary widely to provide for concentrations of the components/reagents that substantially optimize the reactions that need to occur during the assay methods and further to optimize substantially the sensitivity of an assay. Under appropriate circumstances, one or more of the components/reagents in the kit can be provided as a dry powder, such as a lyophilized powder, and the kit may further include excipient(s) for dissolution of the dried reagents; in this manner, a reagent solution having the appropriate concentrations for performing a method or assay in accordance with the present disclosure can be obtained from these components. Positive and/or negative controls may also be included with the kit. In addition, the kit can further include a set of written instructions explaining how to use the kit. A kit of this nature can be used in any of the methods described or otherwise contemplated herein.

### EXAMPLES

Examples are provided hereinbelow. However, the present disclosure is to be understood to not be limited in its application to the specific experimentation, results, and laboratory procedures disclosed herein. Rather, the Examples are simply provided as one of various embodiments and are meant to be exemplary, not exhaustive.

### Example 1: Production of Immunogens Comprising Naloxone and Naltrexone Conjugates

This Example illustrates the production of various conjugates containing naloxone or naltrexone through multiple C-position linkages.

Materials and Equipment: The compounds were purified on a Shimadzu HPLC system equipped with a Silica-bond—C18 reverse phase column. The chemical reactions were monitored by TLC (thin layer chromatography) using Silica gel plates from Analtech Inc. (Newark, DE). The silica gel plates were visualized using UV short wave (254 nm). All chemicals were obtained from Sigma Aldrich (St. Louis, MO), Fluka (Waltham, MA), Thermo Scientific (Waltham, MA), and VWR (Radnor, PA), and were used as received. ¹H NMR was recorded on a Bruker ULTRASHIELD™ 600 MHz spectrometer (Bruker, Billerica, MA). Chemical shifts were reported in parts per million (ppm, δ) and related to tetramethylsilane or with deuterated solvent as internal reference. NMR abbreviations used are: s (singlet), br s (broad singlet), d (doublet), t (triplet), q (quartet), dd (doublet of doublets), dt (doublet of triplets), td (triplet of doublets), ddd (doublet of doublet of doublets), J (coupling constant), Hz (Hertz). ESI-MS spectra were recorded on an Agilent HPLC 1290 single quad system equipped with a 6130 mass detector at Siemens Healthineers analytical facilities (Newark, DE). UV: Carry 60 was used for OD280 and BCA concentration measurements.

The following abbreviations have the meanings set forth below:
BCA assay—Bicinchoninic acid assay
eq.—molar equivalent
g—grams
mg—milligram
mmol, mM—millimole
nm—nanometer
CV—column volume
cm²—square centimeter
DCM—Dichloromethane
MeOH—Methanol
MeOD-d₄—Deuterated Methanol having 4 deuterium atoms (for NMR spectra)
DMF—N,N-Dimethylformamide
SuOH—N-hydroxysuccinimide
NaOAc—Sodium acetate
AcOH—Acetic acid
AcO⁻—Acetate salt
EDAC.HCl—N-(3-Dimethylaminopropyl)-N′-ethylcarbodiimide hydrochloride
TFA—Trifluoroacetic acid
TLC—Thin layer chromatography
Rf—retention factor in TLC analysis
UV—Ultra Violet
v/v—Volume ratio
ESI-MS—Electrospray ionization mass spectrometry
m/z—mass to charge ratio
NMR—Nuclear magnetic resonance
MHz—Megahertz
nG6PDH—native Glucose-6-phosphate dehydrogenase enzyme
β-NADH—Nicotinamide adenine dinucleotide
G₆PDNa₂—lucose-6-phosphate disodium salt
OVA—Ovalbumin isolated from chicken egg white
mcKLH—Mariculture Keyhole Limpet Hemocyanin (ThermoFisher Scientific)
TRIS—Tris(hydroxymethyl)aminomethane
KU—dimeric protein complex of nG₆PDH
RPM—rotations per minute
CFA—Complete Freund's Adjuvant
IFA—Incomplete Freund's Adjuvant
ELISA—Enzyme-Linked Immunosorbant Assay
EIA—Enzyme Immunoassay
PBS—Phosphate Buffered Saline
HRP—Horseradish Peroxidase
TMB—3,3′,5,5′-Tetramethylbenzidine
μL—Microliter
μg—Microgram
V_{H}—Variable Heavy Chain
V_{L}—Variable Light Chain
C_{H}—Constant Heavy Chain
C_{L}—Constant Light Chain
NAD⁺—Oxidized Nicotinamide Adenine Dinucleotide
NADH—Reduced Nicotinamide Adenine Dinucleotide

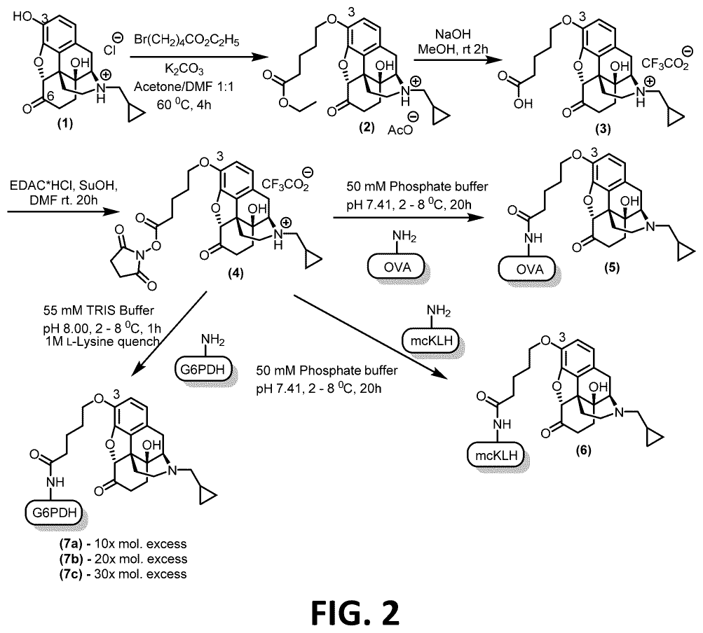

FIG. 2 schematically depicts syntheses of naltrexone hapten, KLH immunogen, and G6PDH conjugates through C-3 position linkage, as described in further detail herein below.

Preparation of Compound (2): In an oven-dried 50 ml round bottom flask equipped with a magnetic stir bar, naltrexone hydrochloride (1) (330 mg, 0.87 mmol) was added into the flask followed by DMF (5 ml), acetone (5 mL), and potassium carbonate (575 mg, 4.16 mmol, 4.78 eq.). The resulting reaction mixture was stirred at room temperature (rt.) for 5 minutes under nitrogen flow; then, ethyl-5-bromovaleric acid (375 μL, 495 mg, 2.36 mmol, ˜2.71 eq.) was added into the mixture. The resulting reaction mixture was placed in an oil bath, and a condenser was attached to the round bottom flask. The reaction mixture was heated at 60° C. under stirring, and the reaction was completed after 4 hours by TLC [(DCM/MeOH 8/2 v/v) Rf₍₁₎=0.36, Rf₍₂₎=0.80]. The solvent was removed on a rotatory evaporator to give a white precipitate which was suspended in acetonitrile (2×10 mL) and filtered-off. The organic extract was concentrated on a rotatory evaporator and further dried overnight (16 h) on an oil pump to remove traces of volatiles. The product was suspended in Acetonitrile/Water (30/70 v/v containing 0.1% Acetic acid) and injected into a Shimadzu HPLC system for purification. Fractions containing the desired product were pooled out, concentrated on a rotatory evaporator, and lyophilized overnight (16-20 hours) to give 474.8 mg of compound (2) as a colorless powder in 93% yield. Compound (2): ESI-MS m/z calcd. for: [C₂₇H₃₆NO₆]⁺ 470.3, found [M+H]⁺=470.3; ¹H NMR (600 MHz, MeOD-d₄) δ: 6.86 (d, J=8.28 Hz, 1H), 6.81 (d, J=8.28 Hz, 1H), 4.97 (br s, 1H), 4.90 (s, 1H), 4.18-4.16 (m, 2H), 4.11 (q, J=7.14 Hz, 3H), 3.45 (d, J=19.74 Hz, 1H), 3.40 (dd, J=13.62; 7.20 Hz, 1H), 3.25 (dd, J=12.69, 4.35 Hz, 1H), 3.19 (dd, J=19.77, 6.27 Hz, 1H), 3.08-3.01 (m, 2H), 2.84 (td, J=13.28, 4.68 Hz, 1H), 2.74 (td, J=12.93, 3.86 Hz, 1H), 2.41-2.38 (m, 2H), 2.25 (dt, J=14.64, 3.00 Hz, 1H), 2.12-2.09 (m, 1H), 1.78-1.74 (m, 4H), 1.72-1.68 (m, 2H), 1.24 (t, J=7.14 Hz, 3H), 1.18-1.15 (m, 1H), 0.86-0.83 (m, 1H), 0.79-0.75 (m, 1H), 0.59-0.50 (m, 2H).

Preparation of compound (3): Compound (2) (474.8 mg, 0.81 mmol) was dissolved in MeOH (5 mL); then, sodium hydroxide 10N solution was added (0.53 mL), and the resulting reaction mixture was stirred at room temperature for 2 hours. The reaction completion was confirmed by checking by TLC [(DCM/MeOH 8/2 v/v), Rf₍₂₎=0.80 Rf₍₃₎=0.20]. The reaction mixture was acidified with HCl (12N, 0.5 mL) on an ice bath. The solvent was removed on a rotatory evaporator. The resulting white cake was suspended in acetonitrile (2 mL) and filtered-off. The filtrate was concentrated on a rotatory evaporator. Then water containing 0.1% TFA was added, and the resulting sample was lyophilized overnight (16-20 hours). After lyophilization, 471 mg of the title compound was obtained as a colorless powder in 100% yield. Compound (3): ESI-MS m/z calcd. for: [C₂₅H₃₂NO₆]⁺ 442.2, found [M+H]⁺=442.2; ¹H NMR (600 MHz, MeOD-d₄) δ: 6.85 (d, J=8.28 Hz, 1H), 6.78 (d, J=8.28 Hz, 1H), 4.86 (s, 1H), 4.20-4.18 (m, 2H), 3.91 (d, J=6.06 Hz, 1H), 3.37 (d, J=19.54 Hz, 1H), 3.19 (dd, J=13.32, 7.08 Hz, 1H), 3.11 (dd, J=12.66, 4.74 Hz, 1H), 3.07-3.06 (m, 1H), 3.02 (dd, J=14.55, 5.13 Hz, 1H), 2.89 (dd, J=13.38, 7.32 Hz, 1H), 2.76 (td, J=13.16, 4.90 Hz, 1H), 2.61 (td, J=12.82, 4.00 Hz, 1H), 2.34-2.32 (m, 2H), 2.26 (dt, J=14.61, 3.14 Hz, 1H), 2.04-2.02 (m, 1H), 1.81-1.76 (m, 4H), 1.70 (td, J=14.31, 3.52 Hz, 1H), 1.65 (dd, J=13.44, 3.00 Hz, 1H), 1.28-1.10 (m, 1H), 0.81-0.78 (m, 1H), 0.75-0.71 (m, 1H), 0.51-0.44 (m, 2H).

Preparation of compound (4): Compound (3) (10.6 mg, 0.019 mmol) was dissolved in degassed DMF (1.06 mL) to make a 10 mg/ml hapten-DMF solution; then, H-hydroxysuccinimide (6.58 mg, 0.057 mmol, 3 eq.) was added. The resulting reaction mixture was stirred until homogenous, and then EDAC.HCl (7.30 mg, 0.038 mmol, 2 eq.) was added. The resulting reaction mixture was stirred at room temperature for 20 hours and monitored by TLC [(DCM/MeOH 8/2 v/v), Rf₍₃₎=0.80, Rf₍₄₎=0.20)]. The formation of compound (4) was confirmed by TLC and MS direct loop: ESI m/z calcd. for: [C₂₉H₃₅N₂O₈]⁺=539.2, found [M+H]⁺=539.3 and [M+Na]⁺=561.3. This compound (4) was used for the next reaction without any further purification.

Preparation of naltrexone OVA (5): OVA, (12 mg, 2.81×10⁻⁴ mmol) was dissolved in 50 mM phosphate buffer, pH 7.41 (2 mL). The resulting protein solution was gentle vortexed until homogenous; then, it was cooled to 0-4° C. using an ice bath. The compound (4)—DMF solution (0.501 mL, 40 eq.) prepared previously was added dropwise under gentle swirling to the protein solution. The resulting reaction mixtures were rocked at 4° C. for 20 hours (overnight), then purified on the G-25M SEPHADEX® column (GE Healthcare, Chicago, IL) (CV=70 cm³, pre-equilibrated with 2 CV of 50 mM phosphate buffer pH 7.00). The conjugate elution was monitored at OD₂₈₀. A sharp peak containing the desired naltrexone OVA conjugate was eluted in the interval 67-90 mL. Naltrexone OVA (5) was collected and concentrated. The concentration of the conjugate was determined by OD₂₈₀ to be 0.89 mg/ml. OVA conjugate (5) was obtained in a total of 9 mg with 75% yield.

Preparation of naltrexone KLH immunogen (6): Naltrexone KLH immunogen (6) was prepared using the same protocol as described for preparation of naltrexone OVA (5). In this case, 20 mg of mcKLH was used, which was treated with compound (4)—DMF solution (0.488 mL, 7×10⁻³ mmol). After purification, the concentration of the conjugate was determined by BCA assay to be 0.96 mg/ml. KLH immunogen (6) was obtained in 15.35 mg with 76.8% yield.

Preparation of native G6PDH for bio-conjugation: Native G6PDH enzyme emulsion (5.5 mL, 5.5 KU, 64.35 mg) was centrifuged at 18,000 RPM, 4° C. for 30 minutes. The resulting clear aqueous layer was discarded and the remaining white cake was suspended in 55 mM TRIS buffer pH 8.00 (2.00 mL). The suspension was gentle swirled until dissolved; then, it was loaded onto a pre-equilibrated G-25M Sephadex column (CV=196 mL) with 55 mM TRIS buffer pH 8.00. The enzyme eluted in the interval 75-116 mL. The enzyme's concentration was adjusted to 5 mg/mL using an Amicon stirring cell concentrator (30,000 MW cut-off); then, it was cooled to 0-4° C. using an ice-bath. Glucose-6-phosphate di-sodium salt [G6PDNa₂] 240 mg was then added. The resulting reaction mixture was gentle swirled to 1 minute; then, β-NADH (120 mg) was added, and the resulting reaction mixture was stirred again for 1 minute. This enzyme solution was equally divided onto three plastic tubes (1.90 mL corresponding to 9.50 mg of enzyme per tube). The tubes 1-3 containing native G6PDHs were stored on an ice bath and ready for compound (4) addition.

Preparation of compound (4) for bio-conjugation: Compound (3) (9.71 mg, 0.017 mmol) was dissolved in degassed DMF (0.242 mL) to make a 40 mg/ml hapten-DMF solution; then, N-hydroxysuccinimide (6.20 mg, 0.054 mmol, ˜3 eq.) was added. The resulting reaction mixture was stirred until homogenous, and then EDAC.HCl (7.30 mg, 0.038 mmol, 2 eq.) was added. The resulting reaction mixture was stirred for 22 hours at room temperature and monitored by TLC [(DCM/MeOH 8/2 v/v), Rf₍₄₎=0.80, Rf₍₃₎=0.20]. After the formation of compound (4), degassed DMF (0.242 μL) was added to make a 20 mg/ml compound (4)-DMF solution. ESI m/z calcd. for: [C₂₉H₃₅N₂O₈]⁺=539.3, found [M+H]⁺=539.3, [M+Na]⁺=561.3; This compound (4) was used for bio-conjugation any without further purification.

Preparation of naltrexone nG6PDH enzyme (7a-c): In tube 1, compound (4) (243 μL—DMF solution) was added [10× molar excess of compound (4) to nG6PDH]. The reaction mixture was rocked at 2-8° C. for 1 hour; then, it was quenched with L-lysine [1M solution, 9 μL, 10× molar excess L-Lysine respective compound (4)]. The reaction mixture was then rocked at room temperature for 20 minutes. This conjugate was purified by a G-50M SEPHADEX® column (CV=67 cm²) pre-equilibrated with 55 mM TRIS buffer pH 7.00 (2 CV). The conjugate was eluted in the interval 45-60 mL as a sharp peak. All fractions containing the desired conjugate were collected and concentrated. The concentration of the conjugate was determined by OD₂₈₀ to be 1.16 mg/mL. The G6PDH conjugate (7a) was obtained in 7.93 mg, with a yield of 83%.

In tube 2, compound (4) (485 μL of—DMF solution) was added (20× molar excess of compound (4) to nG6PDH); the reaction mixture was rocked at 2-8° C. for 1 hour; then, it was quenched with L-lysine [1M solution, 18 μL, 10× molar excess L-Lysine to compound (4)]. The reaction mixture was then rocked at room temperature for 20 minutes. This conjugate was purified by a G-50M SEPHADEX® column (CV=67 cm²) pre-equilibrated with 55 mM TRIS buffer pH 7.00 (2 CV). The conjugate was eluted in the interval 45-60 mL as a sharp peak. All fractions containing the desired conjugate were collected and concentrated. The concentration of the conjugate was determined by OD₂₈₀ to be 1.25 mg/ml. The G6PDH conjugate (7b) was obtained in 8.09 mg, with a yield of 85%.

In tube 3, compound (4) (729 μL—DMF solution) was added [30× molar excess of compound (4) to nG6PDH]. The reaction mixture was rocked at 2-8° C. for 1 hour; then, it was quenched with L-lysine [1M solution, 27 μL, 10× molar excess L-Lysine to compound (4)]. The reaction mixture was then rocked at room temperature for 20 minutes. This conjugate was purified by a G-50M SEPHADEX® column (CV=67 cm²) pre-equilibrated with 55 mM TRIS buffer pH 7.00 (2 CV). The conjugate was eluted in the interval 45-60 mL as a sharp peak. All fractions containing the desired conjugate were collected and concentrated. The concentration of the conjugate was determined by OD₂₈₀ to be 1.28 mg/ml. The G6PDH conjugate (7c) was obtained in 8.27 mg, with a yield of 87%.

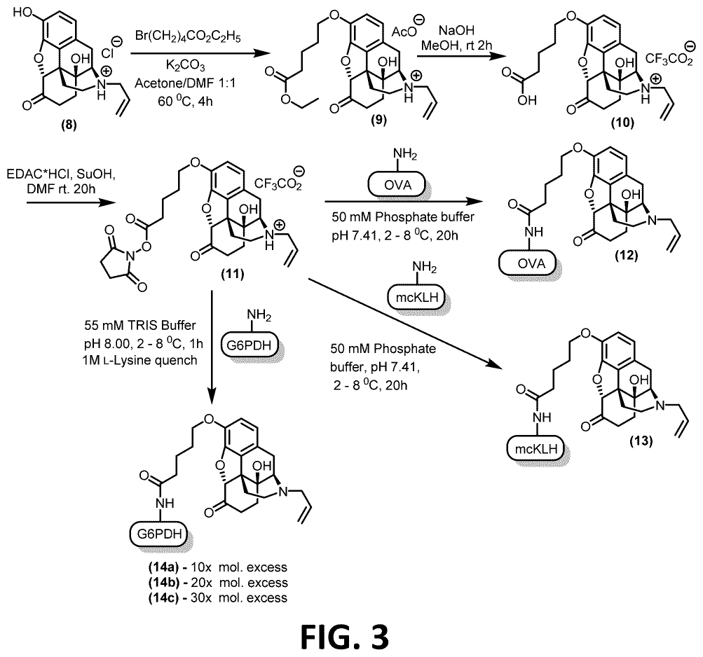

FIG. 3 schematically depicts syntheses of naloxone hapten, KLH immunogen, and G6PDH conjugates through C-3 position linkage, as described in further detail herein below.

Preparation of compound (9): This compound was prepared using the same synthetic method as described for preparation of compound (2). After lyophilization, compound (9) (465.5 mg) was obtained as a colorless powder in 99% yield. Compound (9): ESI-MS m/z calcd. for: [C₂₆H₃₄NO₆]⁺ 456.2, found [M+H]⁺=456.2; 1H NMR (600 MHZ, MeOD-d₄) δ: 6.87 (d, J=8.28 Hz, 1H), 6.82 (d, J=8.28 Hz, 1H), 6.03-5.96 (m, 1H), 5.71 (d, J=16.98 Hz, 1H), 5.64 (d, J=10.26 Hz, 1H), 4.90 (s, 1H), 4.20-4.16 (m, 2H), 4.11 (q, J=7.14 Hz, 2H), 3.98 (dd, J=13.59, 8.62 Hz, 1H), 3.89 (dd, J=13.62, 5.64 Hz, 1H), 3.78 (d, J=6.00 Hz, 1H), 3.48 (d, J=19.80 Hz, 1H), 3.31-3.28 (m, 1H), 3.10 (dd, J=19.80, 6.18 Hz, 1H), 3.03 (td, J=14.65, 4.97 Hz, 1H), 2.87-2.75 (m, 2H), 2.40 (m, 2H), 2.24 (dt, J=14.70, 3.00 Hz, 1H), 2.06-2.02 (m, 1H), 1.78-1.76 (m, 4H), 1.73-1.66 (m, 2H), 1.25 (t, J=7.14 Hz, 3H).

Preparation of compound (10): This compound was prepared using the same synthetic method as described for preparation of compound (3). After lyophilization, compound (10) (447 mg) was obtained as a colorless powder with a yield of nearly 100%. Compound (10): ESI-MS m/z calcd. for: [C₂₄H₃₀NO₆]⁺=428.2, found [M+H]⁺=428.2; ¹H NMR (600 MHz, MeOD-d₄) δ: 6.82 (d, J=8.22 Hz, 1H), 6.75 (d, J=8.22 Hz, 1H), 5.99-5.91 (m, 1H), 5.48 (dd, J=17.13, 1.17 Hz, 1H), 5.43-5.41 (m, 2H), 4.82 (s 1H), 4.18-4.16 (m, 2H), 3.60 (dd, J=13.60, 7.53 Hz, 1H), 3.55 (dd, J=13.59, 6.03 Hz, 1H), 3.42 (d, J=5.88 Hz, 1H), 3.30 (s, 1H), 3.03 (td, J=14.55, 5.10 Hz, 1H), 2.96 (dd, J=12.48, 4.80 Hz, 1H), 2.85 (dd, J=19.32, 6.06 Hz, 1H), 2.65 (td, J=12.99, 5.04 Hz, 1H), 2.46 (td, J=12.63, 3.92 Hz, 1H), 2.36-2.33 (m, 2H), 2.22 (dt, J=14.52, 3.12 Hz, 1H), 1.97-1.94 (m, 1H), 1.80-1.76 (m, 4H), 1.65 (dd, J=14.25, 3.45 Hz, 1H), 1.61-1.58 (m, 1H).

Preparation of compound (11): This compound was prepared using the same synthetic method as described for preparation of compound (4).

Preparation of naloxone OVA (12): This conjugate was prepared using the same synthetic method as described for preparation of OVA conjugate (5).

Preparation of naloxone KLH Immunogen (13): This immunogen was prepared using the same synthetic method as described for preparation of KLH immunogen (6).

Preparation of naloxone nG6PDH [14(a-c)]: These nG6PDH conjugates were prepared using the same synthetic method as described for preparation of nG6PDHs [7(a-c)].

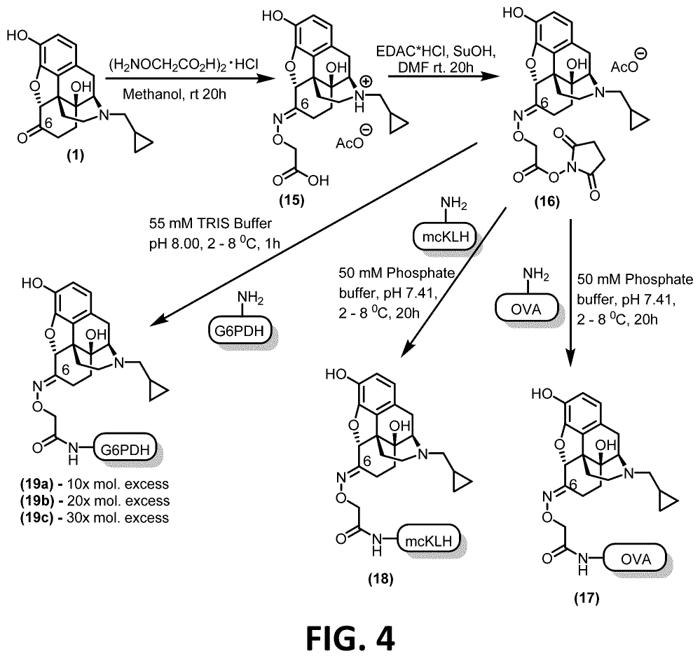

FIG. 4 schematically depicts syntheses of naltrexone hapten, KLH immunogen, and G6PDH conjugates through C-6 position linkage, as described in detail herein below.

Preparation of compound (15): Naltrexone hydrochloride (1) (118 mg, 0.31 mmol) was dissolved in MeOH (7 mL); then, NaOAc (292 mg, 2.24 mmol) was added. The resulting reaction mixture was stirred at room temperature for 5 minutes; then, O-(carboxymethyl)hydroxylamine hemihydrochloride (210.7 mg, 1.92 mmol) was added into the mixture. The resulting reaction mixture was stirred at room temperature overnight (20 hours). TLC analysis of the reaction mixture showed reaction completion [(DCM/MeOH 8/2 v/v) R_{f(1)}=0.4, R_{f(15)}=0.02]. The solvent was removed on a rotatory evaporator to give a white suspension. Then, water/acetonitrile (1.4 mL/0.6 mL v/v) containing 0.1% AcOH was added. The crude reaction mixture was then injected into a preparative Shimadzu HPLC system for purification. Fractions containing the desired product were pooled together, concentrated on a rotatory evaporator, and lyophilized overnight (16-20 hours). This gave the compound (15) (124 mg) as a colorless powder in 75% yield. Compound (15): ESI-MS m/z calcd. for: [C₂₂H₂₇NO₆]⁺ 415.2, found [M+H]⁺=415.2, and [M+Na]⁺=429.2; ¹H NMR (600 MHZ, MeOD-d₄) δ: 6.76 (d, J=8.10 Hz, 1H), 6.72 (d, J=8.22 Hz, 1H), 5.06 (s, 1H), 4.66 (s, 2H), 3.99 (d, J=6.60 Hz, 1H), 3.42 (d, J=19.74 Hz, 1H), 3.38 (dd, J=13.32, 7.03 Hz, 1H), 3.17 (dd, J=12.99, 5.01 Hz, 1H), 3.12 (dd, J=19.86, 6.84 Hz, 1H), 2.97 (dd, J=13.62, 7.50 Hz, 1H), 2.91 (td, J=13.05, 4.04 Hz, 1H), 2.82 (ddd, J=17.76, 6.54, 1.86 Hz, 1H), 2.68 (td, J=13.43, 5.00 Hz, 1H), 2.65-2.59 (m, 1H), 1.80-1.76 (m, 2H), 1.51 (ddd, J=14.25, 12.09, 6.54 Hz, 1H), 1.17-1.10 (m, 1H), 0.88-0.84 (m, 1H), 0.80-0.75 (m, 1H), 0.57-0.50 (m, 2H).

Preparation of compound (16): This compound was prepared using the same synthetic method as described for preparation of compound (4).

Preparation of naltrexone OVA (17): This conjugate was prepared using the same synthetic method as described for preparation of OVA conjugate (5).

Preparation of naltrexone KLH Immunogen (18): This immunogen was prepared using the same synthetic method as described for preparation of KLH immunogen (6).

Preparation of naltrexone nG6PDH [19(a-c)]: These nG6PDH conjugates were prepared using the same synthetic method as described for preparation of nG6PDHs [7(a-c)].

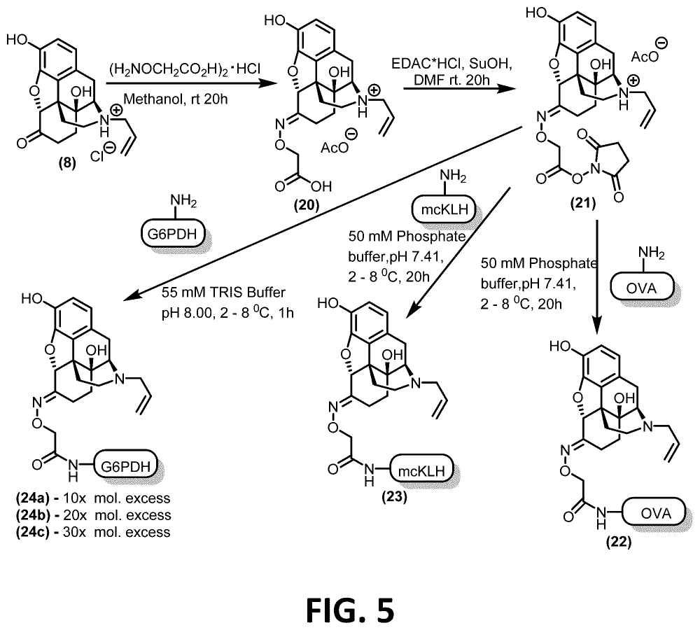

FIG. 5 schematically depicts syntheses of naloxone hapten, KLH immunogen, and G6PDH conjugates through C-6 position linkage, as described in detail herein below.

Preparation of compound (20): Naloxone hydrochloride (8) (110 mg, 0.27 mmol) was dissolved in MeOH (7 mL); then, NaOAc (260 mg, 2.00 mmol) was added. The resulting reaction mixture was stirred at room temperature for 5 minutes; then, O-(carboxymethyl)hydroxylamine hemihydrochloride (186 mg, 1.69 mmol) was added. The resulting reaction mixture was stirred at room temperature overnight. TLC analysis showed the reaction completion (DCM/MeOH 8/2 v/v Rf₍₁₎=0.40, Rf₍₂₀₎=0.02). The reaction mixture formed a white precipitate. The reaction mixture was concentrated on a rotatory evaporator to give a white suspension, which was dissolved in water containing 0.1% AcOH and injected into a Shimadzu HPLC for purification. Fractions containing the desired product were pooled together, concentrated on a rotatory evaporator, and lyophilized overnight (16-20 hours). This gave the compound (20) (125 mg) in 87% yield. Compound (20): ESI-MS m/z calcd. for: [C₂₁H₂₅NO₆]⁺ 401.2, found [M+H]⁺=401.2, [M+Na]⁺=423.2; ¹H NMR (600 MHz, MeOD-d₄) δ: 6.74 (d, J=8.17 Hz, 1H), 6.70 (d, J=8.22 Hz, 1H), 5.98-5.90 (m, 1H), 5.68 (d, J=17.16 Hz, 1H), 5.64 (d, J=10.38 Hz, 1H), 5.04 (s, 1H), 4.64 (s, 2H), 3.95 (dd, J=13.65, 8.67 Hz, 1H), 3.81 (dd, J=13.68, 5.58 Hz, 1H), 3.63 (d, J=6.54 Hz, 1H), 3.43 (d, J=19.74 Hz, 1H), 3.22 (dd, J=12.93, 4.95 Hz, 1H), 3.02 (dd, J=19.80, 6.60 Hz, 1H), 2.92 (td, J=13.13, 4.10 Hz, 1H), 2.80 (ddd, J=17.73, 6.39, 1.89 Hz, 1H), 2.65 (td, J=13.47, 5.04 Hz, 1H), 2.61-2.54 (m, 1H), 1.77 (dd, J=13.68, 3.30 Hz, 1H), 1.71 (ddd, J=14.26, 6.67, 2.38 Hz, 1H), 1.45 (ddd, J=14.26, 12.20, 6.43 Hz, 1H).

Preparation of compound (21): This compound was prepared using the same synthetic method as described for preparation of compound (4).

Preparation of naloxone OVA (22): This conjugate was prepared using the same synthetic method as described for preparation of OVA conjugate (5).

Preparation of naloxone KLH Immunogen (23): This immunogen was prepared using the same synthetic method as described for preparation of KLH immunogen (6).

Preparation of naloxone nG6PDH [24(a-c)]: These nG6PDH conjugates were prepared using the same synthetic method as described for preparation of nG6PDHs [7(a-c)].

### Example 2: Production of Anti-Naloxone and Anti-Naltrexone Monoclonal Antibodies

This Example illustrates the production of specific monoclonal antibodies to naloxone and naltrexone. These monoclonal antibodies can be used in naloxone and naltrexone specific assays, respectively.

In addition, these monoclonal antibodies can be used as blocker antibodies in opiate assays (such as, but not limited to, a hydrocodone assay). It is commonly observed that various opiate assays provide false positive results for patients undergoing naloxone/naltrexone treatment. In such cases, the patients can be screened using the naloxone/naltrexone specific assays disclosed herein to rule out any false positives. In addition (and/or alternatively), biological samples utilized in various opiate assays can be subjected to a pretreatment step with these naloxone/naltrexone-specific monoclonal antibodies to remove any naloxone/naltrexone present in the samples and thus prevent or substantially reduce any naloxone/naltrexone interference observed in quantitative clinical opiate assays.

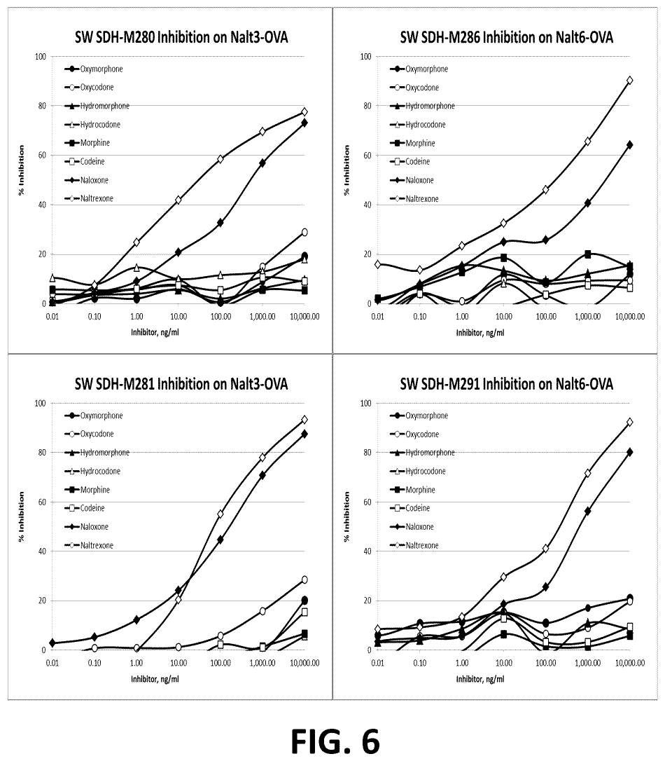

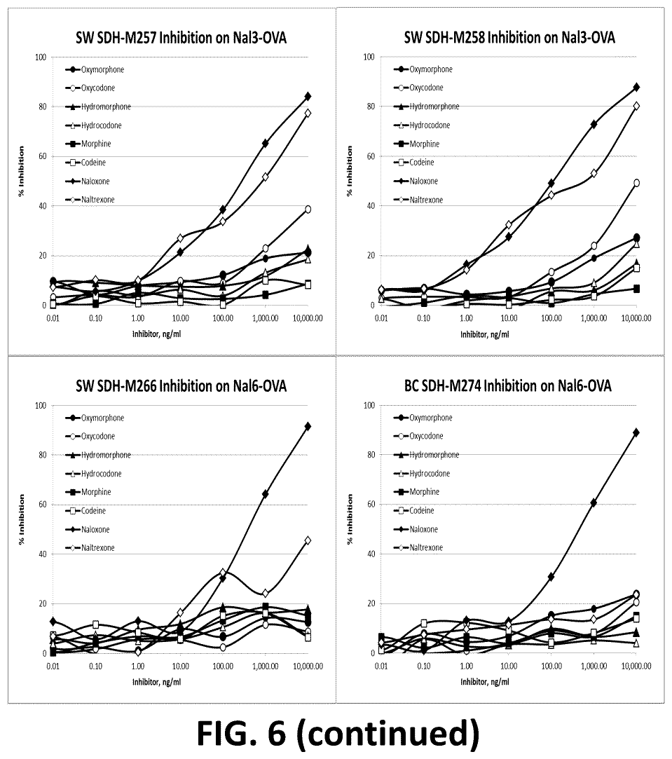

In this Example, various immunogens comprising naloxone or naltrexone attached to KLH at various positions were prepared and used as immunogens to immunize mice (e.g., BALB/c mice, Swiss Webster mice, or an AJ strain of mice) intraperitoneally. Mice were immunized three or more times to generate high titer immune response. Primary immunization was performed using adjuvants like CFA (immunogen emulsified using CFA), followed by subsequent booster immunizations using adjuvants like IFA. Following immunizations, mice were bled, and serum samples were isolated using centrifugation technique. Serum samples from these mice were tested for anti-naloxone and anti-naltrexone antibodies using a conjugate of naloxone and/or naltrexone with ovalbumin (ovalbumin conjugate). A microtiter plate ELISA was employed, and the antibodies were examined for binding to the ovalbumin conjugate and subsequently to free drug molecules using an inhibition ELISA. FIG. 6 shows inhibition ELISA results using eight important opioid drugs. The immunogens used to obtain the mice bleeds shown in FIG. 6 are as follows. Top row, left to right: naltrexone 3-KLH (Formula III, wherein R₂ is a cyclopropyl); naltrexone 6-KLH (Formula VI, wherein R₂ is a cyclopropyl); naloxone 3-KLH (Formula III, wherein R₂ is an allyl); and naloxone 3-KLH. Bottom row, left to right: naltrexone 3-KLH; naltrexone 6-KLH; naloxone 6-KLH (Formula VI, wherein R₂ is an allyl); and naloxone 6-KLH.

Mice with good anti-drug antibody titers and binding to free drug in the ELISA inhibition assays were selected to proceed with generation of monoclonal antibodies, in a similar manner to that described in U.S. Pat. No. 9,815,907 (issued to Sharma et al. on Nov. 14, 2017). In particular, mice with highest titers and best specificity were boosted three days prior to fusion. On the day of fusion, spleen cells were harvested from these mice and were fused with myeloma cell line P3X63Ag8.653 using PEG-assisted fusion protocols. After about ten days, hybridoma supernatants were screened for anti-naloxone and anti- naltrexone antibodies using a plate ELISA (direct binding and inhibition ELISA for specificity). Positive clones were further expanded and sub-cloned, and supernatants were purified using a Protein A SEPHAROSE® (GE Healthcare, Chicago, IL) column. Purified antibody samples were tested using ELISA for binding to the immunogen (i.e., a conjugate of drug-carrier protein) and to free opioids.

Table 1 lists the designations for some of the selected hybridoma clones generated against naloxone and naltrexone drug molecules, as described herein.

### Table 1: List of Monoclonal Antibodies Generated Against Naloxone- and Naltrexone-KLH Conjugates

TABLE 1
List of Monoclonal Antibodies Generated
Against Naloxone- and Naltrexone-KLH Conjugates

| mAb Designation | Drug |
|---|---|
| 179B 9G1.1 | Naloxone |
| 179B 9E12 | Naloxone |
| 179B 10A7 | Naloxone |
| 179A 6H6 | Naloxone |
| 179B 9E7 | Naloxone |
| 180A 3D3 | Naltrexone |
| 180B 13F7 | Naltrexone |
| 180C 1F8 | Naltrexone |
| 180C 7G8 | Naltrexone |
| 180C 8A10 | Naltrexone |
| 180C 9D5 | Naltrexone |
| 180C 9G8 | Naltrexone |
| 180C 2A12 | Naltrexone |

Antibody solution (Immune mouse bleeds or hybridoma clones supernatant or purified antibody) was screened using an inhibition ELISA procedure according to the following protocol. Plates were coated with Naloxone 3-Ovalbumin, Naloxone 6-Ovalbumin, Naltrexone 3-Ovalbumin, or Naltrexone 6-Ovalbumin at 1 μg/mL in phosphate buffered saline at 50 μl per well. Plate coating was performed for 1 hour or more at room temperature or overnight at about 2° C. to 8° C. The plates were then flicked dry and blocked with 200 μL per well of blocking buffer solution (0.5% Casein solution in PBS containing 0.05% TWEEN® 20 surfactant (Croda International PLC, Snaith, United Kingdom). Plate blocking was performed by incubation for 30 minutes or more at room temperature with plate shaking. The plates were washed using a plate washer (BioTek®, Winooski, VT) with plate stacker with the washing buffer being MILLIQ® water (Millipore Corporation, Billerica, MA) containing 0.05% TWEEN® 20. The monoclonal antibody to be screened was then added to each well along with free drug as follows: 25 μL per well of appropriately diluted culture supernatant (or antibody solution) and added 25 μL of 20 μg/ml of free drug. Incubation was performed for about 1 hour at room temperature with plate shaking. The plate was washed. An enzyme conjugate (goat anti-mouse IgG coupled to HRP diluted in blocking buffer diluted to 1:3000) was added at 50 μL per well. Incubation was performed for about 1 hour at room temperature with shaking. The plate was then washed, and a chromogenic solution (TMB from Moss Substrates, Pasadena MD) was added at a volume of 100 μL per well. If a desired antibody was present in the hybridoma supernatant, then a decrease in optical density was observed compared to the well containing no free drug. The plates were read at 650 nm wavelength using an ELISA plate reader (Molecular Devices, San Jose, CA). Note: appropriate dilution of antibody solution was determined by performing a titration ELISA. The antibody dilution was chosen where around 1.0 optical density was observed.

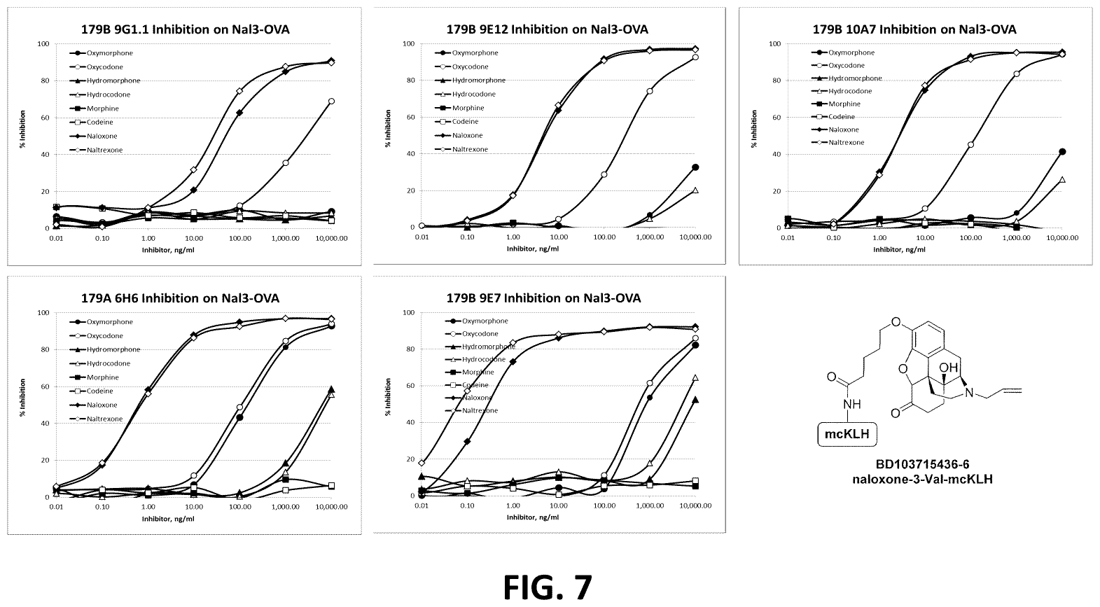

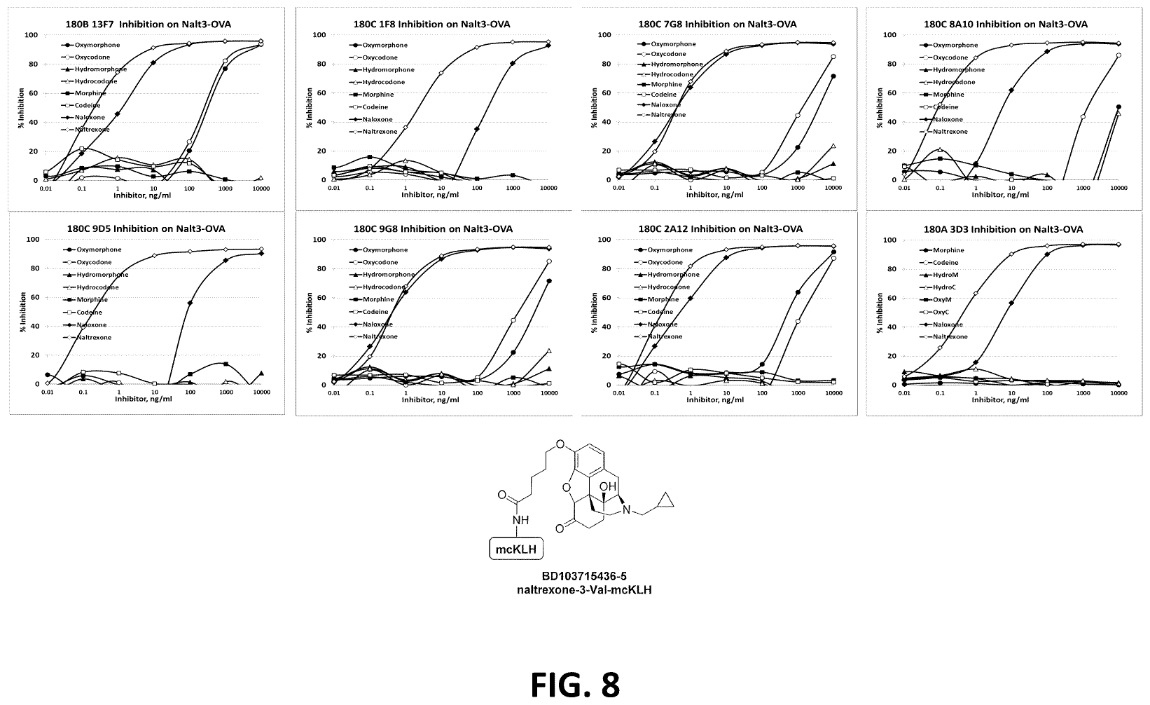

As shown in FIG. 7, monoclonal antibodies designated 179B 9G1.1, 179B 9E12, 179B 10A7, 179A 6H6, and 179B 9E7 were generated using naloxone-3-Val-mcKLH (Formula III, wherein R₂ is an allyl) and possessed good specificity for naloxone over the eight other key opioids tested in the inhibition ELISA assay. As shown in FIG. 8, monoclonal antibodies designated 180B 13F7, 180C 1F8, 180C 7G8, 180C 8A10, 180C 9D5, 180C 9G8, 180C 2A12, and 180A 3D3 were generated using naltrexone-3-Val-mcKLH (Formula III, wherein R₂ is a cyclopropyl) and possessed good specificity for naltrexone over the eight other key opioids tested in the inhibition ELISA assay. Based on these results, the monoclonal antibodies were then used in naloxone/naltrexone specific immunoassays as well as in methods of preventing or substantially reducing naloxone/naltrexone interference in other opiate assays, as described in further detail below.

### Example 3: Monoclonal Antibody Sequencing

Hybridoma cells produced as in Example 2 were used to sequence the various monoclonal antibodies' heavy and light chains. The sequences of different clones were aligned, and the consensus sequences were provided. The various sequences obtained for each monoclonal antibody are shown in Tables 2-14 below.

### Table 2: Sequence Identifiers for Various Sequences of the Anti-Naltrexone Monoclonal Antibody 180C 2A12

TABLE 2
Sequence Identifiers for Various Sequences of the
Anti-Naltrexone Monoclonal Antibody 180C 2A12

| | SEQ ID NO: | SEQ ID NO: |
|---|---|---|
| | of Amino | of DNA |
| Sequence | Acid Sequence | Sequence |
| Heavy Chain | 1 | 9 |
| Heavy Chain CDR1 | 2 | 10 |
| Heavy Chain CDR2 | 3 | 11 |
| Heavy Chain CDR3 | 4 | 12 |
| Light Chain | 5 | 13 |
| Light Chain CDR1 | 6 | 14 |
| Light Chain CDR2 | 7 | 15 |
| Light Chain CDR3 | 8 | 16 |
| Heavy Chain Variable Region | 17 | 19 |
| Light Chain Variable Region | 18 | 20 |

### Table 3: Sequence Identifiers for Various Sequences of the Anti-Naltrexone Monoclonal Antibody 180A 3D3

TABLE 3
Sequence Identifiers for Various Sequences of the
Anti-Naltrexone Monoclonal Antibody 180A 3D3

| | SEQ ID NO: | SEQ ID NO: |
|---|---|---|
| | of Amino | of DNA |
| Sequence | Acid Sequence | Sequence |
| Heavy Chain | 21 | 29 |
| Heavy Chain CDR1 | 22 | 30 |
| Heavy Chain CDR2 | 23 | 31 |
| Heavy Chain CDR3 | 24 | 32 |
| Light Chain | 25 | 33 |
| Light Chain CDR1 | 26 | 34 |
| Light Chain CDR2 | 27 | 35 |
| Light Chain CDR3 | 28 | 36 |
| Heavy Chain Variable Region | 37 | 39 |
| Light Chain Variable Region | 38 | 40 |

### Table 4: Sequence Identifiers for Various Sequences of the Anti-Naltrexone Monoclonal Antibody 180B13F7

TABLE 4
Sequence Identifiers for Various Sequences of the
Anti-Naltrexone Monoclonal Antibody 180B13F7

| | SEQ ID NO: | SEQ ID NO: |
|---|---|---|
| | of Amino | of DNA |
| Sequence | Acid Sequence | Sequence |
| Heavy Chain | 41 | 49 |
| Heavy Chain CDR1 | 42 | 50 |
| Heavy Chain CDR2 | 43 | 51 |
| Heavy Chain CDR3 | 44 | 52 |
| Light Chain | 45 | 53 |
| Light Chain CDR1 | 46 | 54 |
| Light Chain CDR2 | 47 | 55 |
| Light Chain CDR3 | 48 | 56 |
| Heavy Chain Variable Region | 57 | 59 |
| Light Chain Variable Region | 58 | 60 |

### Table 5: Sequence Identifiers for Various Sequences of the Anti-Naltrexone Monoclonal Antibody 180C 1F8

TABLE 5
Sequence Identifiers for Various Sequences of the
Anti-Naltrexone Monoclonal Antibody 180C 1F8

| | SEQ ID NO: | SEQ ID NO: |
|---|---|---|
| | of Amino | of DNA |
| Sequence | Acid Sequence | Sequence |
| Heavy Chain | 61 | 69 |
| Heavy Chain CDR1 | 62 | 70 |
| Heavy Chain CDR2 | 63 | 71 |
| Heavy Chain CDR3 | 64 | 72 |
| Light Chain | 65 | 73 |
| Light Chain CDR1 | 66 | 74 |
| Light Chain CDR2 | 67 | 75 |
| Light Chain CDR3 | 68 | 76 |
| Heavy Chain Variable Region | 77 | 79 |
| Light Chain Variable Region | 78 | 80 |

### Table 6: Sequence Identifiers for Various Sequences of the Anti-Naltrexone Monoclonal Antibody 180C 7G8

TABLE 6
Sequence Identifiers for Various Sequences of the
Anti-Naltrexone Monoclonal Antibody 180C 7G8

| | SEQ ID NO: | SEQ ID NO: |
|---|---|---|
| | of Amino | of DNA |
| Sequence | Acid Sequence | Sequence |
| Heavy Chain | 81 | 89 |
| Heavy Chain CDR1 | 82 | 90 |
| Heavy Chain CDR2 | 83 | 91 |
| Heavy Chain CDR3 | 84 | 92 |
| Light Chain | 85 | 93 |
| Light Chain CDR1 | 86 | 94 |
| Light Chain CDR2 | 87 | 95 |
| Light Chain CDR3 | 88 | 96 |
| Heavy Chain Variable Region | 97 | 99 |
| Light Chain Variable Region | 98 | 100 |

### Table 7: Sequence Identifiers for Various Sequences of the Anti-Naltrexone Monoclonal Antibody 180C 8A10

TABLE 7
Sequence Identifiers for Various Sequences of the
Anti-Naltrexone Monoclonal Antibody 180C 8A10

| | SEQ ID NO: | SEQ ID NO: |
|---|---|---|
| | of Amino | of DNA |
| Sequence | Acid Sequence | Sequence |
| Heavy Chain | 101 | 109 |
| Heavy Chain CDR1 | 102 | 110 |
| Heavy Chain CDR2 | 103 | 111 |
| Heavy Chain CDR3 | 104 | 112 |
| Light Chain | 105 | 113 |
| Light Chain CDR1 | 106 | 114 |
| Light Chain CDR2 | 107 | 115 |
| Light Chain CDR3 | 108 | 116 |
| Heavy Chain Variable Region | 117 | 119 |
| Light Chain Variable Region | 118 | 120 |

### Table 8: Sequence Identifiers for Various Sequences of the Anti-Naltrexone Monoclonal Antibody 180C 9D5

TABLE 8
Sequence Identifiers for Various Sequences of the
Anti-Naltrexone Monoclonal Antibody 180C 9D5

| | SEQ ID NO: | SEQ ID NO: |
|---|---|---|
| | of Amino | of DNA |
| Sequence | Acid Sequence | Sequence |
| Heavy Chain | 121 | 129 |
| Heavy Chain CDR1 | 122 | 130 |
| Heavy Chain CDR2 | 123 | 131 |
| Heavy Chain CDR3 | 124 | 132 |
| Light Chain | 125 | 133 |
| Light Chain CDR1 | 126 | 134 |
| Light Chain CDR2 | 127 | 135 |
| Light Chain CDR3 | 128 | 136 |
| Heavy Chain Variable Region | 137 | 139 |
| Light Chain Variable Region | 138 | 140 |

### Table 9: Sequence Identifiers for Various Sequences of the Anti-Naltrexone Monoclonal Antibody 180C 9G8

TABLE 9
Sequence Identifiers for Various Sequences of the
Anti-Naltrexone Monoclonal Antibody 180C 9G8

| | SEQ ID NO: | SEQ ID NO: |
|---|---|---|
| | of Amino | of DNA |
| Sequence | Acid Sequence | Sequence |
| Heavy Chain | 141 | 149 |
| Heavy Chain CDR1 | 142 | 150 |
| Heavy Chain CDR2 | 143 | 151 |
| Heavy Chain CDR3 | 144 | 152 |
| Light Chain | 145 | 153 |
| Light Chain CDR1 | 146 | 154 |
| Light Chain CDR2 | 147 | 155 |
| Light Chain CDR3 | 148 | 156 |
| Heavy Chain Variable Region | 157 | 159 |
| Light Chain Variable Region | 158 | 160 |

### Table 10: Sequence Identifiers for Various Sequences of the Anti-Naloxone Monoclonal Antibody 179B 9G1.1

TABLE 10
Sequence Identifiers for Various Sequences of the
Anti-Naloxone Monoclonal Antibody 179B 9G1.1

| | SEQ ID NO: | SEQ ID NO: |
|---|---|---|
| | of Amino | of DNA |
| Sequence | Acid Sequence | Sequence |
| Heavy Chain | 161 | 169 |
| Heavy Chain CDR1 | 162 | 170 |
| Heavy Chain CDR2 | 163 | 171 |
| Heavy Chain CDR3 | 164 | 172 |
| Light Chain | 165 | 173 |
| Light Chain CDR1 | 166 | 174 |
| Light Chain CDR2 | 167 | 175 |
| Light Chain CDR3 | 168 | 176 |
| Heavy Chain Variable Region | 177 | 179 |
| Light Chain Variable Region | 178 | 180 |

### Table 11: Sequence Identifiers for Various Sequences of the Anti-Naloxone Monoclonal Antibody 179B 9E12

TABLE 11
Sequence Identifiers for Various Sequences of the
Anti-Naloxone Monoclonal Antibody 179B 9E12

| | SEQ ID NO: | SEQ ID NO: |
|---|---|---|
| | of Amino | of DNA |
| Sequence | Acid Sequence | Sequence |
| Heavy Chain | 181 | 189 |
| Heavy Chain CDR1 | 182 | 190 |
| Heavy Chain CDR2 | 183 | 191 |
| Heavy Chain CDR3 | 184 | 192 |
| Light Chain | 185 | 193 |
| Light Chain CDR1 | 186 | 194 |
| Light Chain CDR2 | 187 | 195 |
| Light Chain CDR3 | 188 | 196 |
| Heavy Chain Variable Region | 197 | 199 |
| Light Chain Variable Region | 198 | 200 |

### Table 12: Sequence Identifiers for Various Sequences of the Anti-Naloxone Monoclonal Antibody 179B 10A7

TABLE 12
Sequence Identifiers for Various Sequences of the
Anti-Naloxone Monoclonal Antibody 179B 10A7

| | SEQ ID NO: | SEQ ID NO: |
|---|---|---|
| | of Amino | of DNA |
| Sequence | Acid Sequence | Sequence |
| Heavy Chain | 201 | 209 |
| Heavy Chain CDR1 | 202 | 210 |
| Heavy Chain CDR2 | 203 | 211 |
| Heavy Chain CDR3 | 204 | 212 |
| Light Chain | 205 | 213 |
| Light Chain CDR1 | 206 | 214 |
| Light Chain CDR2 | 207 | 215 |
| Light Chain CDR3 | 208 | 216 |
| Heavy Chain Variable Region | 217 | 219 |
| Light Chain Variable Region | 218 | 220 |

### Table 13: Sequence Identifiers for Various Sequences of the Anti-Naloxone Monoclonal Antibody 179A 6H6

TABLE 13
Sequence Identifiers for Various Sequences of the
Anti-Naloxone Monoclonal Antibody 179A 6H6

| | SEQ ID NO: | SEQ ID NO: |
|---|---|---|
| | of Amino | of DNA |
| Sequence | Acid Sequence | Sequence |
| Heavy Chain | 221 | 229 |
| Heavy Chain CDR1 | 222 | 230 |
| Heavy Chain CDR2 | 223 | 231 |
| Heavy Chain CDR3 | 224 | 232 |
| Light Chain | 225 | 233 |
| Light Chain CDR1 | 226 | 234 |
| Light Chain CDR2 | 227 | 235 |
| Light Chain CDR3 | 228 | 236 |
| Heavy Chain Variable Region | 237 | 239 |
| Light Chain Variable Region | 238 | 240 |

Table 14: Sequence Identifiers for Various Sequences of the Anti-Naloxone Monoclonal Antibody 179B 9E7

TABLE 14
Sequence Identifiers for Various Sequences of the
Anti-Naloxone Monoclonal Antibody 179B 9E7

| | SEQ ID NO: | SEQ ID NO: |
|---|---|---|
| | of Amino | of DNA |
| Sequence | Acid Sequence | Sequence |
| Heavy Chain | 241 | 249 |
| Heavy Chain CDR1 | 242 | 250 |
| Heavy Chain CDR2 | 243 | 251 |
| Heavy Chain CDR3 | 244 | 252 |
| Light Chain | 245 | 253 |
| Light Chain CDR1 | 246 | 254 |
| Light Chain CDR2 | 247 | 255 |
| Light Chain CDR3 | 248 | 256 |
| Heavy Chain Variable Region | 257 | 259 |
| Light Chain Variable Region | 258 | 260 |

### Example 4: Use of Monoclonal Antibodies in Naloxone and Naltrexone-Specific Assays

In this Example, monoclonal antibodies produced in Example 2 were utilized in naloxone-and naltrexone-specific immunoassays to demonstrate the ability of these monoclonal antibodies to detect one or both drugs in a clinical immunoassay.

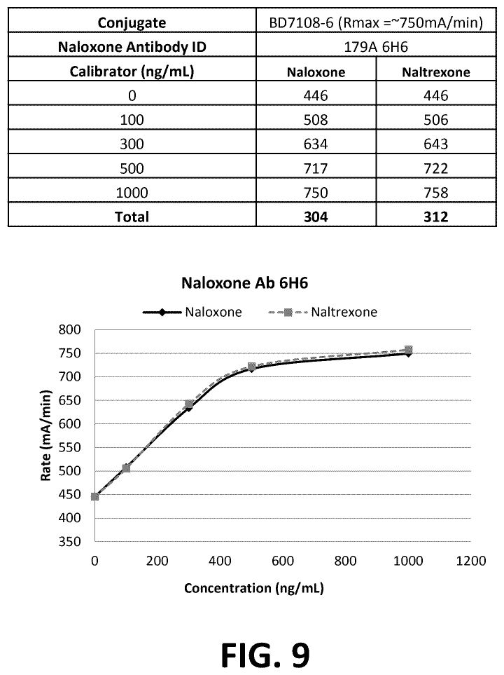

FIG. 9 graphically depicts naloxone and naltrexone specific assays using the anti-naloxone monoclonal antibody 179A 6H6. As can be seen, the anti-naloxone monoclonal antibody detects both naloxone and naltrexone at substantially the same specificity. Similar results were also obtained with anti-naloxone monoclonal antibodies 179B 10A7, 179B 9G1, 179B 9E12, and 179B 9E7. Therefore, these antibodies could be used in a clinical detection immunoassay for either or both drugs.

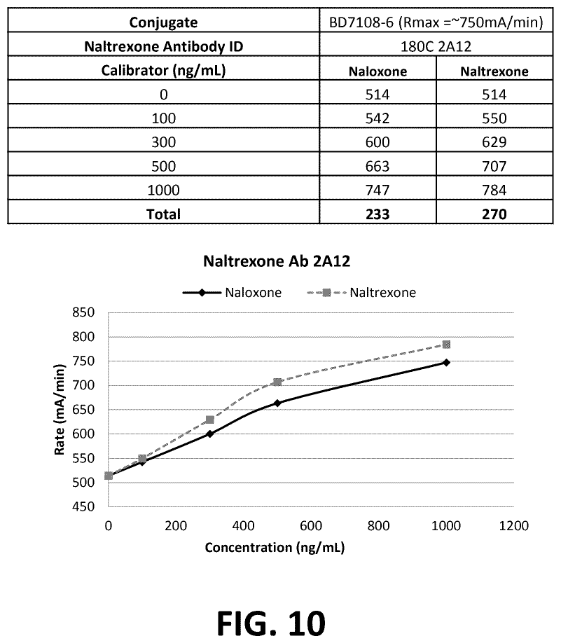

FIG. 10 graphically depicts naloxone and naltrexone specific assays using the anti-naltrexone monoclonal antibody 180C 2A12. This anti-naltrexone monoclonal antibody was also capable of detecting naloxone in addition to naltrexone, albeit at a lower specificity compared to naltrexone. However, this antibody could still be used in a clinical detection immunoassay for either or both drugs.

Based on these results, these antibodies can be utilized in an assay structure as follows. An EMIT® II (enzyme multiplied immunoassay, Siemens Healthineers, Newark, DE) Plus Naloxone and/or Naltrexone Assay is a homogeneous enzyme immunoassay technique used for the analysis of specific compounds in human urine. The assay is based on competition between drug in the specimen and drug labeled with glucose-6-phosphate dehydrogenase (rG6PDH) for antibody binding sites. Enzyme activity decreases upon binding to the antibody, so the drug concentration in the specimen can be measured in terms of enzyme activity. Active enzyme converts nicotinamide adenine dinucleotide (NAD) to NADH in the presence of glucose-6-phosphate (G6P), resulting in an absorbance change that is measured spectrophotometrically. Endogenous serum G6PDH does not interfere because the coenzyme NAD functions only with the bacterial (Leuconostoc mesenteroides) enzyme employed in the assay.

The EMIT® assay format comprises an antibody/substrate reagent, known as reagent 1. This reagent includes the antibody as well as buffers, preservatives, and stabilizers for the assay. The EMIT® assay format also comprises an enzyme reagent, known as reagent 2. This reagent includes the conjugate as well as buffers, preservatives, and stabilizers for the assay.

### Example 5: Reduction of Naloxone/Naltrexone Interference in Opioid Assays

In this Example, monoclonal antibodies produced in Example 2 were utilized in opiate enzyme multiplied immunoassays to demonstrate the ability to reduce naloxone/naltrexone interference in these assays.

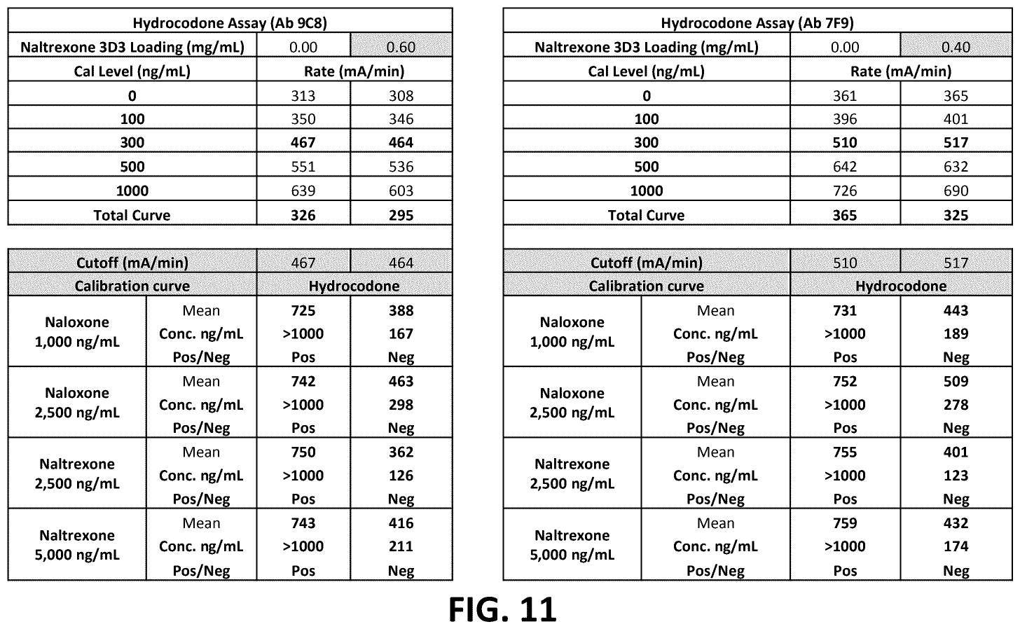

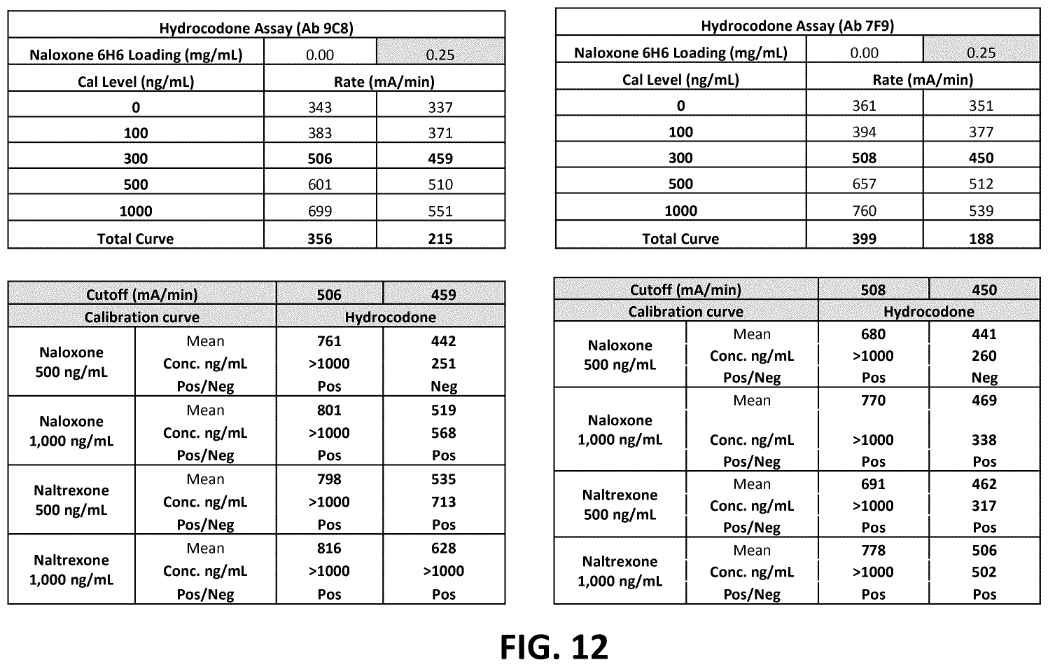

FIG. 11 demonstrates that, in the presence of the naltrexone antibody blocker 3D3, the cross reactivity of naloxone and naltrexone changes from a positive to a negative response to the cutoff (300 ng/ml). Similarly, FIG. 12 demonstrates that, in the presence of the naloxone antibody blocker 6H6, the cross reactivity of naloxone changes from a positive to a negative response to the cutoff (300 ng/ml), only at a much lower concentration.

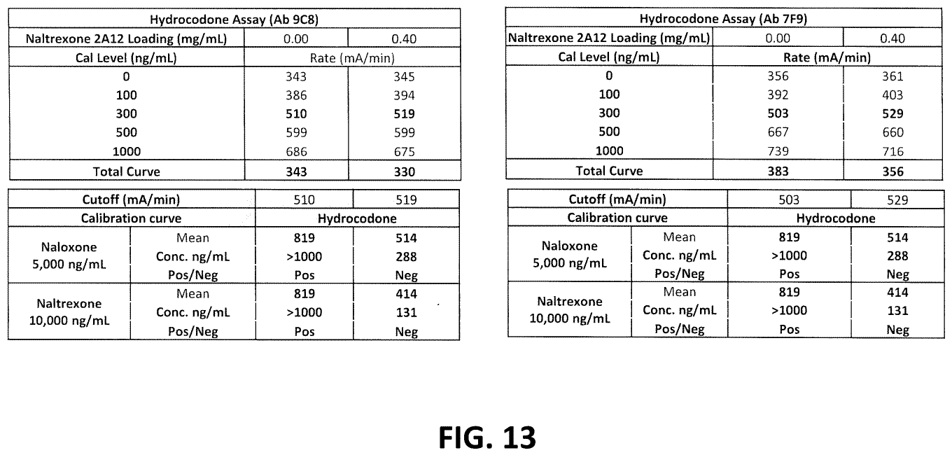

In FIG. 13, the anti-naltrexone monoclonal antibody 2A12 was used as a blocker in a hydrocodone assay to increase the amount of naloxone and naltrexone that produces a negative response to the cutoff (300 ng/ml).

Thus, in accordance with the present disclosure, there have been provided compositions and methods of producing and using same which fully satisfy the objectives and advantages set forth hereinabove. Although the present disclosure has been described in conjunction with the specific drawings, experimentation, results, and language set forth hereinabove, it is evident that many alternatives, modifications, and variations will be apparent to those skilled in the art. Accordingly, it is intended to embrace all such alternatives, modifications, and variations that fall within the spirit and broad scope of the present disclosure.

What is claimed is:

1. A method of detecting naloxone and/or naltrexone present in a biological sample, the method comprising the steps of:
contacting the biological sample with an antibody or functional fragment thereof under conditions whereby an antibody/naloxone and/or antibody/naltrexone complex is formed if naloxone and/or naltrexone, respectively, is present in the biological sample; and
detecting any antibody/naloxone and/or antibody/naltrexone complex formed, wherein an amount of antibody/naloxone and/or antibody/naltrexone complex formed is directly proportional to an amount of naloxone and/or naltrexone present in the biological sample, wherein the antibody or functional fragment thereof comprises any one of Formulas III-VIII: 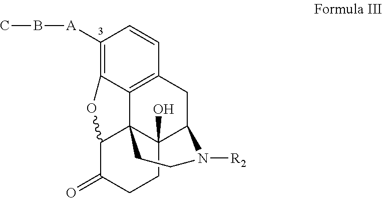
wherein, in Formula III: A is a hetero atom selected from the group consisting of —O—, —N—, or —S—; B is a functional group selected from the group consisting of —CO—, a lower alkyl (C₀ to C₁₀), —CONH—, —SO₂—, or —PO₄—; C is a carrier protein; and R₂ is H, alkyl, allyl, or methylenecyclopropyl; 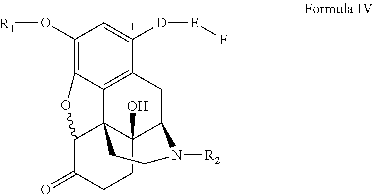
wherein, in Formula IV: D is a hetero atom selected from the group consisting of O—, S—, CO—, or CH₂—; E is a lower alkyl, CO₂—, CONH—, SO₂—, or PO₄—; F is a carrier protein; R₂ is H, alkyl, allyl, or methylenecyclopropyl; and R₁ is H or CH₃; 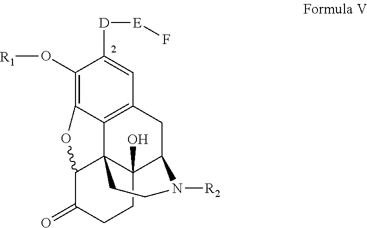
wherein, in Formula V: D is a hetero atom selected from the group consisting of O—, S—, CO—, or CH₂—; E is a lower alkyl, CO₂—, CONH—, SO₂—, or PO₄—; F is a carrier protein; R₂ is H, alkyl, allyl, or methylenecyclopropyl; and R₁ is H or CH₃; 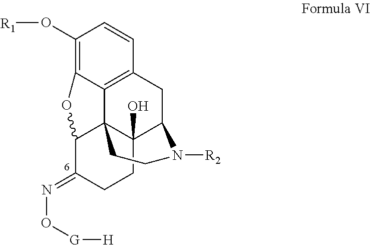
wherein, in Formula VI: G is a linking group which includes at least two of the following: a lower alkyl chain, CO₂—, CONH—, SO₂—, and PO₄—; H is a carrier protein; R₁ is H or CH₃; and R₂ is H, alkyl, allyl, or methylenecyclopropyl; 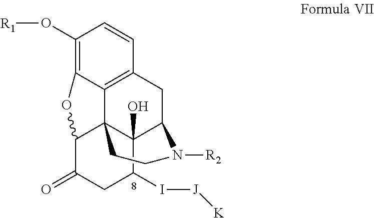
wherein, in Formula VII: I is a hetero atom selected from the group consisting of O—, S—, CO—, or CH₂—; J is a lower alkyl chain, CO₂—, CONH—, SO₂—, or PO₄—; K is a carrier protein; R₁ is H or CH₃; and R₂ is H, alkyl, allyl, or methylenecyclopropyl; and 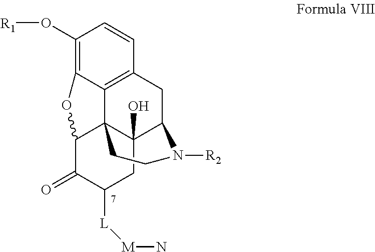
wherein, in Formula VII: L is a hetero atom selected from the group consisting of O, S, CO, or CH₂; M is a lower alkyl chain, CO₂—, CONH—, SO₂—, or PO₄—; N is a carrier protein; R₁ is H or CH₃; and R₂ is H, alkyl, allyl, or methylenecyclopropyl.

2. The method of claim 1, wherein the antibody or functional fragment thereof has a label attached thereto that is used in the detection of the antibody/naloxone and/or antibody/naltrexone complex.

3. A method of substantially reducing naloxone and/or naltrexone interference from at least one opiate assay of a biological sample, the method comprising the steps of:
contacting the biological sample with at least one antibody or functional fragment thereof of claim 1 under conditions whereby an antibody/naloxone and/or antibody/naltrexone complex is formed if naloxone and/or naltrexone, respectively, is present in the sample;
removing any antibody/naloxone and/or antibody/naltrexone complex formed; and
performing at least one opiate assay on the biological sample in which naloxone and/or naltrexone has been substantially reduced.

4. The method of claim 3, wherein the opiate to be assayed is selected from the group consisting of codeine, hydrocodone, hydromorphone, morphine, oxycodone, oxymorphone, heroin (6-acetylmorphine), and combinations thereof.

5. The method of claim 3, wherein the opiate assay is selected from the group consisting of an ELISA, a chip assay, LC/MS/MS, an immunoassay, an enzyme immunoassay, an enzyme multiplied immunoassay, a fluorescence polarization immunoassay, and combinations thereof.

6. The method of claim 3, wherein the at least one antibody or functional fragment thereof comprises at least one antibody or functional fragment thereof that specifically binds naloxone and at least one antibody or functional fragment thereof that specifically binds naltrexone.


SEQUENCE LISTING (260 sequences)

SEQ ID NO: 1 - protein, 460 aa, synthetic construct
  REGION 1..460: note = Amino Acid sequence of heavy chain of monoclonal antibody SHC 004
MGWSWIFLFLLSGTAGVHCQVQLQQSGPELVKPGGSVKLSCKASGYIFNDYTIHWMKQSPGQGLEWIGWMNRRSFNTMYNDKFKGKATMTVDKSSSTAYIQLSSLTSDDSAVYFCVRDVYDNLDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPGK

SEQ ID NO: 2 - protein, 5 aa, synthetic construct
  REGION 1..5: note = Amino acid sequence of Heavy Chain CDR1 of monoclonal antibody SHC 004
DYTIH

SEQ ID NO: 3 - protein, 17 aa, synthetic construct
  REGION 1..17: note = Amino acid sequence of Heavy Chain CDR2 of monoclonal antibody SHC 004
WMNRRSFNTMYNDKFKG

SEQ ID NO: 4 - protein, 8 aa, synthetic construct
  REGION 1..8: note = Amino acid sequence of Heavy Chain CDR3 of monoclonal antibody SHC 004
DVYDNLDY

SEQ ID NO: 5 - protein, 238 aa, synthetic construct
  REGION 1..238: note = Amino acid sequence of Light Chain of monoclonal antibody SHC 004
MKLPVRLLVLMFWIPASSSDVLMTQTPLSLPVSLGDQASISCRSSQSIVHSNGNTYLEWYLQKPGQSPRLLIYKVSNRFSGVPDRFSGSGSGTDFTLRISRVEAEDLGLYYCFQGSHVPLTFGAGTKLELKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 6 - protein, 16 aa, synthetic construct
  REGION 1..16: note = Amino acid sequence of Light Chain CDR1 of monoclonal antibody SHC 004
RSSQSIVHSNGNTYLE

SEQ ID NO: 7 - protein, 7 aa, synthetic construct
  REGION 1..7: note = Amino acid sequence of Light Chain CDR2 of monoclonal antibody SHC 004
KVSNRFS

SEQ ID NO: 8 - protein, 9 aa, synthetic construct
  REGION 1..9: note = Amino acid sequence of Light Chain CDR3 of monoclonal antibody SHC 004
FQGSHVPLT

SEQ ID NO: 9 - protein, 1383 aa, synthetic construct
  REGION 1..1383: note = DNA sequence of Heavy Chain of monoclonal antibody SHC 004
ATGGGGTGGAGCTGGATCTTTCTCTTCCTCCTGTCAGGAACTGCAGGTGTCCATTGCCAGGTCCAGCTGCAGCAGTCTGGACCTGAGCTGGTGAAGCCTGGAGGTTCAGTGAAGCTGTCCTGCAAGGCTTCTGGCTACATCTTCAATGACTATACTATACACTGGATGAAGCAGAGTCCTGGACAGGGACTTGAGTGGATTGGATGGATGAATCGTAGAAGTTTTAACACCATGTACAATGACAAGTTCAAGGGCAAGGCCACAATGACTGTAGACAAATCCTCCAGCACAGCCTACATTCAACTCAGCAGCCTGACCTCTGACGACTCTGCGGTCTATTTCTGTGTAAGAGATGTTTATGATAATCTGGACTACTGGGGTCAAGGAACCTCAGTCACCGTCTCCTCAGCCAAAACGACACCCCCATCTGTCTATCCACTGGCCCCTGGATCTGCTGCCCAAACTAACTCCATGGTGACCCTGGGATGCCTGGTCAAGGGCTATTTCCCTGAGCCAGTGACAGTGACCTGGAACTCTGGATCCCTGTCCAGCGGTGTGCACACCTTCCCAGCTGTCCTGCAGTCTGACCTCTACACTCTGAGCAGCTCAGTGACTGTCCCCTCCAGCACCTGGCCCAGCGAGACCGTCACCTGCAACGTTGCCCACCCGGCCAGCAGCACCAAGGTGGACAAGAAAATTGTGCCCAGGGATTGTGGTTGTAAGCCTTGCATATGTACAGTCCCAGAAGTATCATCTGTCTTCATCTTCCCCCCAAAGCCCAAGGATGTGCTCACCATTACTCTGACTCCTAAGGTCACGTGTGTTGTGGTAGACATCAGCAAGGATGATCCCGAGGTCCAGTTCAGCTGGTTTGTAGATGATGTGGAGGTGCACACAGCTCAGACGCAACCCCGGGAGGAGCAGTTCAACAGCACTTTCCGCTCAGTCAGTGAACTTCCCATCATGCACCAGGACTGGCTCAATGGCAAGGAGTTCAAATGCAGGGTCAACAGTGCAGCTTTCCCTGCCCCCATCGAGAAAACCATCTCCAAAACCAAAGGCAGACCGAAGGCTCCACAGGTGTACACCATTCCACCTCCCAAGGAGCAGATGGCCAAGGATAAAGTCAGTCTGACCTGCATGATAACAGACTTCTTCCCTGAAGACATTACTGTGGAGTGGCAGTGGAATGGGCAGCCAGCGGAGAACTACAAGAACACTCAGCCCATCATGGACACAGATGGCTCTTACTTCATCTACAGCAAGCTCAATGTGCAGAAGAGCAACTGGGAGGCAGGAAATACTTTCACCTGCTCTGTGTTACATGAGGGCCTGCACAACCACCATACTGAGAAGAGCCTCTCCCACTCTCCTGGTAAATGA

SEQ ID NO: 10 - DNA, 15 bp, synthetic construct
  misc_feature 1..15: note = DNA sequence of Heavy Chain CDR1 of monoclonal antibody SHC 004
  source 1..15: mol_type = other DNA
gactatactatacac

SEQ ID NO: 11 - DNA, 51 bp, synthetic construct
  misc_feature 1..51: note = DNA sequence of Heavy Chain CDR2 of monoclonal antibody SHC 004
  source 1..51: mol_type = other DNA
tggatgaatcgtagaagttttaacaccatgtacaatgacaagttcaagggc

SEQ ID NO: 12 - DNA, 24 bp, synthetic construct
  misc_feature 1..24: note = DNA sequence of Heavy Chain CDR3 of monoclonal antibody SHC 004
  source 1..24: mol_type = other DNA
gatgtttatgataatctggactac

SEQ ID NO: 13 - DNA, 717 bp, synthetic construct
  misc_feature 1..717: note = DNA sequence of Light Chain of monoclonal antibody SHC 004
  source 1..717: mol_type = other DNA
atgaagttgcctgttaggctgttggtgctgatgttctggattcctgcttccagcagtgatgttttgatgacccaaactccactctccctgcctgtcagtcttggagatcaagcctccatctcttgcagatccagtcagagtattgtacatagtaatggaaacacctatttagagtggtacctgcagaaaccaggccagtctccaaggctcctgatctacaaagtttccaaccgattttctggggtcccagacaggttcagtggcagtggatccgggacagatttcacactcaggatcagtagagtggaggctgaggatctgggactttattactgctttcaaggttcacacgttcctctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccagagacatcaatgtcaagtggaagattgatggcagtgaacgacaaaatggtgtcctgaacagttggactgatcaggacagcaaagacagcacctacagcatgagcagcaccctcacattgaccaaggacgagtatgaacgacataacagctatacctgtgaggccactcacaagacatcaacttcacccatcgtcaagagcttcaacaggaatgagtgttag

SEQ ID NO: 14 - DNA, 48 bp, synthetic construct
  misc_feature 1..48: note = DNA sequence of Light Chain CDR1 of monoclonal antibody SHC 004
  source 1..48: mol_type = other DNA
agatccagtcagagtattgtacatagtaatggaaacacctatttagag

SEQ ID NO: 15 - DNA, 21 bp, synthetic construct
  misc_feature 1..21: note = DNA sequence of Light Chain CDR2 of monoclonal antibody SHC 004
  source 1..21: mol_type = other DNA
aaagtttccaaccgattttct

SEQ ID NO: 16 - DNA, 27 bp, synthetic construct
  misc_feature 1..27: note = DNA sequence of Light Chain CDR3 of monoclonal antibody SHC 004
  source 1..27: mol_type = other DNA
tttcaaggttcacacgttcctctcacg

SEQ ID NO: 17 - protein, 117 aa, synthetic construct
  REGION 1..117: note = Amino acid sequence of Heavy Chain variable region of monoclonal antibody SHC 004
QVQLQQSGPELVKPGGSVKLSCKASGYIFNDYTIHWMKQSPGQGLEWIGWMNRRSFNTMYNDKFKGKATMTVDKSSSTAYIQLSSLTSDDSAVYFCVRDVYDNLDYWGQGTSVTVSS

SEQ ID NO: 18 - protein, 112 aa, synthetic construct
  REGION 1..112: note = Amino acid sequence of Light Chain variable region of monoclonal antibody SHC 004
DVLMTQTPLSLPVSLGDQASISCRSSQSIVHSNGNTYLEWYLQKPGQSPRLLIYKVSNRFSGVPDRFSGSGSGTDFTLRISRVEAEDLGLYYCFQGSHVPLTFGAGTKLELK

SEQ ID NO: 19 - DNA, 351 bp, synthetic construct
  misc_feature 1..351: note = DNA sequence of Heavy Chain variable region of monoclonal antibody SHC 004
  source 1..351: mol_type = other DNA
caggtccagctgcagcagtctggacctgagctggtgaagcctggaggttcagtgaagctgtcctgcaaggcttctggctacatcttcaatgactatactatacactggatgaagcagagtcctggacagggacttgagtggattggatggatgaatcgtagaagttttaacaccatgtacaatgacaagttcaagggcaaggccacaatgactgtagacaaatcctccagcacagcctacattcaactcagcagcctgacctctgacgactctgcggtctatttctgtgtaagagatgtttatgataatctggactactggggtcaaggaacctcagtcaccgtctcctca

SEQ ID NO: 20 - DNA, 336 bp, synthetic construct
  misc_feature 1..336: note = DNA sequence of Light Chain variable region of monoclonal antibody SHC 004
  source 1..336: mol_type = other DNA
gatgttttgatgacccaaactccactctccctgcctgtcagtcttggagatcaagcctccatctcttgcagatccagtcagagtattgtacatagtaatggaaacacctatttagagtggtacctgcagaaaccaggccagtctccaaggctcctgatctacaaagtttccaaccgattttctggggtcccagacaggttcagtggcagtggatccgggacagatttcacactcaggatcagtagagtggaggctgaggatctgggactttattactgctttcaaggttcacacgttcctctcacgttcggtgctgggaccaagctggagctgaaa

SEQ ID NO: 21 - protein, 137 aa, synthetic construct
  REGION 1..137: note = Amino Acid sequence of heavy chain of monoclonal antibody 180A 3D3
MYLGLNCVFIVFLLKGVQSEVNLEESGGGLVQPGGSMKLSCVASGFTFSNYWMNWVRQSPERGLEWVAEISLKSYNYATHYAESVKGRFTISGDESKNSVYLQMNNLRAEDTGIYFCTRGDSYALDYGQRTLVTVSS

SEQ ID NO: 22 - protein, 5 aa, synthetic construct
  REGION 1..5: note = Amino Acid sequence of heavy chain CDR1 of monoclonal antibody 180A 3D3
NYWMN

SEQ ID NO: 23 - protein, 19 aa, synthetic construct
  REGION 1..19: note = Amino Acid sequence of heavy chain CDR2 of monoclonal antibody 180A 3D3
EISLKSYNYATHYAESVKG

SEQ ID NO: 24 - protein, 8 aa, synthetic construct
  REGION 1..8: note = Amino Acid sequence of heavy chain CDR3 of monoclonal antibody 180A 3D3
GDSYALDY

SEQ ID NO: 25 - protein, 132 aa, synthetic construct
  REGION 1..132: note = Amino Acid sequence of light chain of monoclonal antibody 180A 3D3
MEFQTQVLMSLLLCMSGACADIVMTQSPTFLAVTVSKKVTISCTASESLYSSKHKVHYLAWYQKKPEQSPKLLIYGASNRYIGVPDRFTGSGSGTDFTLTISSVQVEDLTHYYCAQFYSYPYTFGGGKLEIK

SEQ ID NO: 26 - protein, 17 aa, synthetic construct
  REGION 1..17: note = Amino Acid sequence of light chain CDR1 of monoclonal antibody 180A 3D3
TASESLYSSKHKVHYLA

SEQ ID NO: 27 - protein, 7 aa, synthetic construct
  REGION 1..7: note = Amino Acid sequence of light chain CDR2 of monoclonal antibody 180A 3D3
GASNRYI

SEQ ID NO: 28 - protein, 9 aa, synthetic construct
  REGION 1..9: note = Amino Acid sequence of light chain CDR3 of monoclonal antibody 180A 3D3
AQFYSYPYT

SEQ ID NO: 29 - DNA, 411 bp, synthetic construct
  misc_feature 1..411: note = DNA sequence of heavy chain of monoclonal antibody 180A 3D3
  source 1..411: mol_type = other DNA
atgtacttgggactgaactgtgtattcatagtttttctcttaaaaggtgtccagagtgaagtaaaccttgaggagtctggaggaggcttggtgcaacctggaggatccatgaaactctcctgtgttgctctggattcactttcagtaactactggatgaactgggtccgccagtctccagagagggggcttgagtgggttgctgaaattagcttgaaatcttataactatgcaacacattatgcggagtctgtgaagggaggttcaccatctcaggagatgaatccaaaaatagtgtctacctgcaaatgaacaacttaagagctgaagatactggcatttatttctgtaccaggggagattcctatgctctggactactgggtcaaagaaccttagtcaccgtctcctca

SEQ ID NO: 30 - DNA, 15 bp, synthetic construct
  misc_feature 1..15: note = DNA sequence of heavy chain CDR1 of monoclonal antibody 180A 3D3
  source 1..15: mol_type = other DNA
aactactggatgaac

SEQ ID NO: 31 - DNA, 56 bp, synthetic construct
  misc_feature 1..56: note = DNA sequence of heavy chain CDR2 of monoclonal antibody 180A 3D3
  source 1..56: mol_type = other DNA
gaaattagcttgaaatcttataactatgcaacacattatgcggagtctgtgaaggg

SEQ ID NO: 32 - DNA, 24 bp, synthetic construct
  misc_feature 1..24: note = DNA sequence of heavy chain CDR3 of monoclonal antibody 180A 3D3
  source 1..24: mol_type = other DNA
ggagattcctatgctctggactac

SEQ ID NO: 33 - DNA, 396 bp, synthetic construct
  misc_feature 1..396: note = DNA sequence of light chain of monoclonal antibody 180A 3D3
  source 1..396: mol_type = other DNA
atggagtttcagacccaggtactcatgtccctgctgctctgcatgtctggtgcctgtgcagacattgtgatgactcagtctccaactttccttgctgtgacagtaagtaagaaggtcaccattagttcacggccagtgagagcctttattcaagcaaacacaaggtgcactacttggcttggtaccagaagaaaccagagcaatctcctaaactgctgatatacggggcatccaaccgatacattggggtccctatcgcttcacaggcagtggatctgggacagatttcactctgaccatcagcagtgtacaggttgaagacctcacacattattactgtgcacagttttacagctatccgtacacgttcggaggggggacaagctggaaataaaa

SEQ ID NO: 34 - DNA, 51 bp, synthetic construct
  misc_feature 1..51: note = DNA sequence of light chain CDR1 of monoclonal antibody 180A 3D3
  source 1..51: mol_type = other DNA
acggccagtgagagcctttattcaagcaaacacaaggtgcactacttggct

SEQ ID NO: 35 - DNA, 21 bp, synthetic construct
  misc_feature 1..21: note = DNA sequence of light chain CDR2 of monoclonal antibody 180A 3D3
  source 1..21: mol_type = other DNA
ggggcatccaaccgatacatt

SEQ ID NO: 36 - DNA, 27 bp, synthetic construct
  misc_feature 1..27: note = DNA sequence of light chain CDR3 of monoclonal antibody 180A 3D3
  source 1..27: mol_type = other DNA
gcacagttttacagctatccgtacacg

SEQ ID NO: 37 - protein, 118 aa, synthetic construct
  REGION 1..118: note = Amino Acid sequence of heavy chain variable region of monoclonal antibody 180A 3D3
EVNLEESGGGLVQPGGSMKLSCVASGFTFSNYWMNWVRQSPERGLEWVAEISLKSYNYATHYAESVKGRFTISGDESKNSVYLQMNNLRAEDTGIYFCTRGDSYALDYGQRTLVTVSS

SEQ ID NO: 38 - protein, 112 aa, synthetic construct
  REGION 1..112: note = Amino Acid sequence of light chain variable region of monoclonal antibody 180A 3D3
DIVMTQSPTFLAVTVSKKVTISCTASESLYSSKHKVHYLAWYQKKPEQSPKLLIYGASNRYIGVPDRFTGSGSGTDFTLTISSVQVEDLTHYYCAQFYSYPYTFGGGKLEIK

SEQ ID NO: 39 - DNA, 284 bp, synthetic construct
  misc_feature 1..284: note = DNA sequence of heavy chain variable region of monoclonal antibody 180A 3D3
  source 1..284: mol_type = other DNA
ctctggattcactttcagtaactactggatgaactgggtccgccagtctccagagagggggcttgagtgggttgctgaaattagcttgaaatcttataactatgcaacacattatgcggagtctgtgaagggaggttcaccatctcaggagatgaatccaaaaatagtgtctacctgcaaatgaacaacttaagagctgaagatactggcatttatttctgtaccaggggagattcctatgctctggactactgggtcaaagaaccttagtcaccgtctcctca

SEQ ID NO: 40 - DNA, 336 bp, synthetic construct
  misc_feature 1..336: note = DNA sequence of light chain variable region of monoclonal antibody 180A 3D3
  source 1..336: mol_type = other DNA
gacattgtgatgactcagtctccaactttccttgctgtgacagtaagtaagaaggtcaccattagttcacggccagtgagagcctttattcaagcaaacacaaggtgcactacttggcttggtaccagaagaaaccagagcaatctcctaaactgctgatatacggggcatccaaccgatacattggggtccctatcgcttcacaggcagtggatctgggacagatttcactctgaccatcagcagtgtacaggttgaagacctcacacattattactgtgcacagttttacagctatccgtacacgttcggaggggggacaagctggaaataaaa

SEQ ID NO: 41 - protein, 135 aa, synthetic construct
  REGION 1..135: note = Amino Acid sequence of heavy chain of monoclonal antibody 180B 13F7
MRVLILVYLLTALPGILSDIQLQESGPGLVKPSQTVSLTCTVTGYSITYVNHWWNWIRQVSGNKLEWIGYISSSGSIDSNPSLKSRIAITRDTSKNQLFLQLKSVTTEDVGTYYCVSEDSSGYHYWGGTTLTVSS

SEQ ID NO: 42 - protein, 7 aa, synthetic construct
  REGION 1..7: note = Amino Acid sequence of heavy chain CDR1 of monoclonal antibody 180B 13F7
YVNHWWN

SEQ ID NO: 43 - protein, 16 aa, synthetic construct
  REGION 1..16: note = Amino Acid sequence of heavy chain CDR2 of monoclonal antibody 180B 13F7
YISSSGSIDSNPSLKS

SEQ ID NO: 44 - protein, 8 aa, synthetic construct
  REGION 1..8: note = Amino Acid sequence of heavy chain CDR3 of monoclonal antibody 180B 13F7
EDSSGYHY

SEQ ID NO: 45 - protein, 131 aa, synthetic construct
  REGION 1..131: note = Amino Acid sequence of light chain of monoclonal antibody 180B 13F7
MKLPVRLLVLMFWIPASSSDVVMTQTPLSLPVSLGDQASISCRSSQSLVFSNGKTYFHWYLQKPGQSPKLLIYRVSNRFSGVPDRFSGSGSGTDFTLKISRVEAEDLGVYFCSQSTHVPWTFGGGTKLEIK

SEQ ID NO: 46 - protein, 16 aa, synthetic construct
  REGION 1..16: note = Amino Acid sequence of light chain CDR1 of monoclonal antibody 180B 13F7
RSSQSLVFSNGKTYFH

SEQ ID NO: 47 - protein, 7 aa, synthetic construct
  REGION 1..7: note = Amino Acid sequence of light chain CDR2 of monoclonal antibody 180B 13F7
RVSNRFS

SEQ ID NO: 48 - protein, 9 aa, synthetic construct
  REGION 1..9: note = Amino Acid sequence of light chain CDR3 of monoclonal antibody 180B 13F7
SQSTHVPWT

SEQ ID NO: 49 - DNA, 405 bp, synthetic construct
  misc_feature 1..405: note = DNA sequence of heavy chain of monoclonal antibody 180B 13F7
  source 1..405: mol_type = other DNA
atgagagtgttgattcttgtgtacctgttgacagcccttcctggtatcttgtctgatatacagcttcaggagtcaggacctggcctggtgaagccttctcagacagtgtccctcacctgcactgtcatggctactctatcacttatgttaatcactggtggaactggatccggcaggtttcaggaaacaaactggagtggatagggtacataagttccagtggcagtattgacagcaatccatctctcaaaagtgaatcgccatcactagagacacttccaagaaccagttattcttgcagttgaaatctgtgactactgaagatgtgggcacatattactgtgtaagcgaagacagctcgggataccactactggggccaggcactactctcacagtctcctca

SEQ ID NO: 50 - DNA, 21 bp, synthetic construct
  misc_feature 1..21: note = DNA sequence of heavy chain CDR1 of monoclonal antibody 180B 13F7
  source 1..21: mol_type = other DNA
tatgttaatcactggtggaac

SEQ ID NO: 51 - DNA, 48 bp, synthetic construct
  misc_feature 1..48: note = DNA sequence of heavy chain CDR2 of monoclonal antibody 180B 13F7
  source 1..48: mol_type = other DNA
tacataagttccagtggcagtattgacagcaatccatctctcaaaagt

SEQ ID NO: 52 - DNA, 24 bp, synthetic construct
  misc_feature 1..24: note = DNA sequence of heavy chain CDR3 of monoclonal antibody 180B 13F7
  source 1..24: mol_type = other DNA
gaagacagctcgggataccactac

SEQ ID NO: 53 - DNA, 390 bp, synthetic construct
  misc_feature 1..390: note = DNA sequence of light chain of monoclonal antibody 180B 13F7
  source 1..390: mol_type = other DNA
atgaagttgcctgttaggctgttggtgctgatgttctggattcctgcttccagtagtgatgttgtgatgacccaaactccactctccctgcctgtcagtcttggagatcaagcctccatctcttgcagtctagtcagagccttgtattcagtaatggaaaaacctatttccattggtacctgcagaagccaggccagtctccaaagctcctgatctacagagtttccaaccgattttctggggtcccagacaggtcagtggcagtggatcagggacagatttcacactcaagatcagcagagtggaggctgaggatctgggagtttatttctgctctcaaagtacacatgttccgtggacgttcggtggaggcaccaagctgaaatcaaa

SEQ ID NO: 54 - DNA, 47 bp, synthetic construct
  misc_feature 1..47: note = DNA sequence of light chain CDR1 of monoclonal antibody 180B 13F7
  source 1..47: mol_type = other DNA
agtctagtcagagccttgtattcagtaatggaaaaacctatttccat

SEQ ID NO: 55 - DNA, 21 bp, synthetic construct
  misc_feature 1..21: note = DNA sequence of light chain CDR2 of monoclonal antibody 180B 13F7
  source 1..21: mol_type = other DNA
agagtttccaaccgattttct

SEQ ID NO: 56 - DNA, 27 bp, synthetic construct
  misc_feature 1..27: note = DNA sequence of light chain CDR3 of monoclonal antibody 180B 13F7
  source 1..27: mol_type = other DNA
tctcaaagtacacatgttccgtggacg

SEQ ID NO: 57 - protein, 117 aa, synthetic construct
  REGION 1..117: note = Amino Acid sequence of heavy chain variable region of monoclonal antibody 180B 13F7
DIQLQESGPGLVKPSQTVSLTCTVTGYSITYVNHWWNWIRQVSGNKLEWIGYISSSGSIDSNPSLKSRIAITRDTSKNQLFLQLKSVTTEDVGTYYCVSEDSSGYHYWGGTTLTVSS

SEQ ID NO: 58 - protein, 112 aa, synthetic construct
  REGION 1..112: note = Amino Acid sequence of light chain variable region of monoclonal antibody 180B 13F7
DVVMTQTPLSLPVSLGDQASISCRSSQSLVFSNGKTYFHWYLQKPGQSPKLLIYRVSNRFSGVPDRFSGSGSGTDFTLKISRVEAEDLGVYFCSQSTHVPWTFGGGTKLEIK

SEQ ID NO: 59 - DNA, 405 bp, synthetic construct; identical to SEQ ID NO: 49
  misc_feature 1..405: note = DNA sequence of heavy chain variable region of monoclonal antibody 180B 13F7
  source 1..405: mol_type = other DNA

SEQ ID NO: 60 - DNA, 333 bp, synthetic construct
  misc_feature 1..333: note = DNA sequence of light chain variable region of monoclonal antibody 180B 13F7
  source 1..333: mol_type = other DNA
gatgttgtgatgacccaaactccactctccctgcctgtcagtcttggagatcaagcctccatctcttgcagtctagtcagagccttgtattcagtaatggaaaaacctatttccattggtacctgcagaagccaggccagtctccaaagctcctgatctacagagtttccaaccgattttctggggtcccagacaggtcagtggcagtggatcagggacagatttcacactcaagatcagcagagtggaggctgaggatctgggagtttatttctgctctcaaagtacacatgttccgtggacgttcggtggaggcaccaagctgaaatcaaa

SEQ ID NO: 61 - protein, 134 aa, synthetic construct
  REGION 1..134: note = Amino Acid sequence of heavy chain of monoclonal antibody 180C 1F8
MEWSWIFLFFLSVTTGVHSQVQLQQSDAELVKPGTSVKISCKASGFTFTDHAIHWVKQRPEQGLEWIGYISPGNGDIKYNEKFKGKATLTADKSSSTAYMQLNSLTSEDSAVYFCDYYNGRVDYWGQTTLTVSS

SEQ ID NO: 62 - protein, 5 aa, synthetic construct
  REGION 1..5: note = Amino Acid sequence of heavy chain CDR1 of monoclonal antibody 180C 1F8
DHAIH

SEQ ID NO: 63 - protein, 17 aa, synthetic construct
  REGION 1..17: note = Amino Acid sequence of heavy chain CDR2 of monoclonal antibody 180C 1F8
YISPGNGDIKYNEKFKG

SEQ ID NO: 64 - protein, 7 aa, synthetic construct
  REGION 1..7: note = Amino Acid sequence of heavy chain CDR3 of monoclonal antibody 180C 1F8
YNGRVDY

SEQ ID NO: 65 - protein, 127 aa, synthetic construct
  REGION 1..127: note = Amino Acid sequence of light chain of monoclonal antibody 180C 1F8
MEFHTQVFVFVFLWLSGVDGDIVMTQSQKFMTTSVGDRVSITCKASQNVGSAVAWYQQKPGQSPKLLIYSASNRFTGVPDRFTGSGSRTDFTLTISNMQSDDLADYFCHQYRSYPYTFGGGTKLEIK

SEQ ID NO: 66 - protein, 11 aa, synthetic construct
  REGION 1..11: note = Amino Acid sequence of light chain CDR1 of monoclonal antibody 180C 1F8
KASQNVGSAVA

SEQ ID NO: 67 - protein, 7 aa, synthetic construct
  REGION 1..7: note = Amino Acid sequence of light chain CDR2 of monoclonal antibody 180C 1F8
SASNRFT

SEQ ID NO: 68 - protein, 9 aa, synthetic construct
  REGION 1..9: note = Amino Acid sequence of light chain CDR3 of monoclonal antibody 180C 1F8
HQYRSYPYT

SEQ ID NO: 69 - DNA, 402 bp, synthetic construct
  misc_feature 1..402: note = DNA sequence of heavy chain of monoclonal antibody 180C 1F8
  source 1..402: mol_type = other DNA
atggaatggagctggatctttctcttcttcctgtcagtaactacaggtgtccactcccaggttcagctgcaacagtctgacgctgagttggtgaaacctgggacttcagtgaagatatcctgcaaggttctggcttcaccttcactgaccatgctattcactgggtgaagcagaggcctgaacagggcctggaatggattggatatatttctcccggaaatggtgatattaagtataatgagaaattcaagggcaggccacactgactgcagacaaatcctccagcactgcctacatgcagctcaacagtctgacatctgaagattctgcagtgtatttctgtgattactacaatggtagggttgactactggggccaaggaccactctcacagtctcctca

SEQ ID NO: 70 - DNA, 15 bp, synthetic construct
  misc_feature 1..15: note = DNA sequence of heavy chain CDR1 of monoclonal antibody 180C 1F8
  source 1..15: mol_type = other DNA
gaccatgctattcac

SEQ ID NO: 71 - DNA, 51 bp, synthetic construct
  misc_feature 1..51: note = DNA sequence of heavy chain CDR2 of monoclonal antibody 180C 1F8
  source 1..51: mol_type = other DNA
tatatttctcccggaaatggtgatattaagtataatgagaaattcaagggc

SEQ ID NO: 72 - DNA, 21 bp, synthetic construct
  misc_feature 1..21: note = DNA sequence of heavy chain CDR1 of monoclonal antibody 180C 1F8
  source 1..21: mol_type = other DNA
tacaatggtagggttgactac

SEQ ID NO: 73 - DNA, 379 bp, synthetic construct
  misc_feature 1..379: note = DNA sequence of light chain of monoclonal antibody 180C 1F8
  source 1..379: mol_type = other DNA
atggagtttcatactcaggtctttgtattcgtgtttctctggttgtctggtgttgatggagacattgtgatgacccagtctcaaaagttcatgaccacgtcagtaggagacagggtcagtatcacctcaaggccagtcagaatgtgggctctgctgtagcctggtatcaacagaaaccaggacaatctcctaaacttctgatttactcagcatccaatcggttcactggagtccctgatcgcttcacaggcagtgatctcggacagatttcactctcaccattagcaatatgcagtctgatgacctggcagattatttctgtcaccaatataggagctatccgtacacgttcggaggggggaccaagctggaaataaaa

SEQ ID NO: 74 - DNA, 33 bp, synthetic construct
  misc_feature 1..33: note = DNA sequence of light chain CDR1 of monoclonal antibody 180C 1F8
  source 1..33: mol_type = other DNA
aaggccagtcagaatgtgggctctgctgtagcc

SEQ ID NO: 75 - DNA, 21 bp, synthetic construct
  misc_feature 1..21: note = DNA sequence of light chain CDR2 of monoclonal antibody 180C 1F8
  source 1..21: mol_type = other DNA
tcagcatccaatcggttcact

SEQ ID NO: 76 - DNA, 27 bp, synthetic construct
  misc_feature 1..27: note = DNA sequence of light chain CDR3 of monoclonal antibody 180C 1F8
  source 1..27: mol_type = other DNA
caccaatataggagctatccgtacacg

SEQ ID NO: 77 - protein, 115 aa, synthetic construct
  REGION 1..115: note = Amino Acid sequence of heavy chain variable region of monoclonal antibody 180C 1F8
QVQLQQSDAELVKPGTSVKISCKASGFTFTDHAIHWVKQRPEQGLEWIGYISPGNGDIKYNEKFKGKATLTADKSSSTAYMQLNSLTSEDSAVYFCDYYNGRVDYWGQTTLTVSS

SEQ ID NO: 78 - protein, 107 aa, synthetic construct
  REGION 1..107: note = Amino Acid sequence of light chain variable region of monoclonal antibody 180C 1F8
DIVMTQSQKFMTTSVGDRVSITCKASQNVGSAVAWYQQKPGQSPKLLIYSASNRFTGVPDRFTGSGSRTDFTLTISNMQSDDLADYFCHQYRSYPYTFGGGTKLEIK

SEQ ID NO: 79 - DNA, 345 bp, synthetic construct
  misc_feature 1..345: note = DNA sequence of heavy chain variable region of monoclonal antibody 180C 1F8
  source 1..345: mol_type = other DNA
caggttcagctgcaacagtctgacgctgagttggtgaaacctgggacttcagtgaagatatcctgcaaggttctggcttcaccttcactgaccatgctattcactgggtgaagcagaggcctgaacagggcctggaatggattggatatatttctcccggaaatggtgatattaagtataatgagaaattcaagggcaggccacactgactgcagacaaatcctccagcactgcctacatgcagctcaacagtctgacatctgaagattctgcagtgtatttctgtgattactacaatggtagggttgactactggggccaaggaccactctcacagtctcctca

SEQ ID NO: 80 - DNA, 319 bp, synthetic construct
  misc_feature 1..319: note = DNA sequence of light chain variable region of monoclonal antibody 180C 1F8
  source 1..319: mol_type = other DNA
gacattgtgatgacccagtctcaaaagttcatgaccacgtcagtaggagacagggtcagtatcacctcaaggccagtcagaatgtgggctctgctgtagcctggtatcaacagaaaccaggacaatctcctaaacttctgatttactcagcatccaatcggttcactggagtccctgatcgcttcacaggcagtgatctcggacagatttcactctcaccattagcaatatgcagtctgatgacctggcagattatttctgtcaccaatataggagctatccgtacacgttcggaggggggaccaagctggaaataaaa

SEQ ID NO: 81 - protein, 136 aa, synthetic construct
  REGION 1..136: note = Amino acid sequence of heavy chain of monoclonal antibody 180C 7G8
MYLGLNCVIIVFLLKGVQSEVKLEESGGGLVQPGGSVKLSCAVFGFTFSDPWMDWVRQSPEKGLEWVAEIRNKAYNHATYYAESVKGRFTISRDDSNSRVSLQMNNLRAEDTGVYYCTAGDSSHSYWQGTLVTVSA

SEQ ID NO: 82 - protein, 5 aa, synthetic construct
  REGION 1..5: note = Amino acid sequence of heavy chain CDR1 of monoclonal antibody 180C 7G8
DPWMD

SEQ ID NO: 83 - protein, 19 aa, synthetic construct
  REGION 1..19: note = Amino acid sequence of heavy chain CDR2 of monoclonal antibody 180C 7G8
EIRNKAYNHATYYAESVKG

SEQ ID NO: 84 - protein, 7 aa, synthetic construct
  REGION 1..7: note = Amino acid sequence of heavy chain CDR3 of monoclonal antibody 180C 7G8
GDSSHSY

SEQ ID NO: 85 - protein, 127 aa, synthetic construct
  REGION 1..127: note = Amino acid sequence of light chain of monoclonal antibody 180C 7G8
MEFQTQVFVFVLLWLSGVDGDIVMTQSQKFMSTSVGDRVSIICKASQNVRTAVAWYQQKPGQSPKTLIYLASNRHTGVPDRFTGSGSGTDFTLTISNVQAEDLADYFCLQHWNYPLTFGGGTKLEIK

SEQ ID NO: 86 - protein, 11 aa, synthetic construct
  REGION 1..11: note = Amino acid sequence of light chain CDR1 of monoclonal antibody 180C 7G8
KASQNVRTAVA

SEQ ID NO: 87 - protein, 7 aa, synthetic construct
  REGION 1..7: note = Amino acid sequence of light chain CDR2 of monoclonal antibody 180C 7G8
LASNRHT

SEQ ID NO: 88 - protein, 9 aa, synthetic construct
  REGION 1..9: note = Amino acid sequence of light chain CDR3 of monoclonal antibody 180C 7G8
LQHWNYPLT

SEQ ID NO: 89 - DNA, 408 bp, synthetic construct
  misc_feature 1..408: note = DNA sequence of heavy chain of monoclonal antibody 180C 7G8
  source 1..408: mol_type = other DNA
atgtacttgggactgaactgtgtaatcatagtttttctcttaaaaggtgtccagagtgaagtgaaacttgaggagtctggaggaggcttggtgcaacctggaggatccgtgaaactctcttgtgctgttttggattcactttcagtgatccctggatggactgggtccgccagtcgccagagaaggggcttgagtgggttgctgaaattagaaacaaagcttataatcatgccacatattatgctgagtctgttaagggaggttcaccatctcaagagatgattccaacagtagagtgtccctgcaaatgaacaacttaagagctgaagacactggcgtttattactgtaccgccggtgactccagtcattcttattggggcaagggactctggtcactgtctctgca

SEQ ID NO: 90 - DNA, 15 bp, synthetic construct
  misc_feature 1..15: note = DNA sequence of heavy chain CDR1 of monoclonal antibody 180C 7G8
  source 1..15: mol_type = other DNA
gatccctggatggac

SEQ ID NO: 91 - DNA, 56 bp, synthetic construct
  misc_feature 1..56: note = DNA sequence of heavy chain CDR2 of monoclonal antibody 180C 7G8
  source 1..56: mol_type = other DNA
gaaattagaaacaaagcttataatcatgccacatattatgctgagtctgttaaggg

SEQ ID NO: 92 - DNA, 21 bp, synthetic construct
  misc_feature 1..21: note = DNA sequence of heavy chain CDR3 of monoclonal antibody 180C 7G8
  source 1..21: mol_type = other DNA
ggtgactccagtcattcttat

SEQ ID NO: 93 - DNA, 379 bp, synthetic construct
  misc_feature 1..379: note = DNA sequence of light chain of monoclonal antibody 180C 7G8
  source 1..379: mol_type = other DNA
atggagtttcagacccaggtctttgtattcgtgttgctctggttgtctggtgttgatggagacattgtgatgacccagtctcaaaaattcatgtccacatcagtaggagacagggtcagcatcatctcaaggccagtcagaatgttcgtactgctgtagcctggtatcaacagaaaccagggcagtctcctaaaacactgatttacttggcatccaaccggcacactggagtccctgatcgcttcacaggcagtgatctgggacagatttcactctcaccattagcaatgtacaagctgaagacctggcagattatttctgtctgcaacattggaattatcctctcacgttcggaggggggaccaaactggaaataaaa

SEQ ID NO: 94 - DNA, 33 bp, synthetic construct
  misc_feature 1..33: note = DNA sequence of light chain CDR1 of monoclonal antibody 180C 7G8
  source 1..33: mol_type = other DNA
aaggccagtcagaatgttcgtactgctgtagcc

SEQ ID NO: 95 - DNA, 21 bp, synthetic construct
  misc_feature 1..21: note = DNA sequence of light chain CDR2 of monoclonal antibody 180C 7G8
  source 1..21: mol_type = other DNA
ttggcatccaaccggcacact

SEQ ID NO: 96 - DNA, 27 bp, synthetic construct
  misc_feature 1..27: note = DNA sequence of light chain CDR3 of monoclonal antibody 180C 7G8
  source 1..27: mol_type = other DNA
ctgcaacattggaattatcctctcacg

SEQ ID NO: 97 - protein, 117 aa, synthetic construct
  REGION 1..117: note = Amino acid sequence of heavy chain variable region of monoclonal antibody 180C 7G8
EVKLEESGGGLVQPGGSVKLSCAVFGFTFSDPWMDWVRQSPEKGLEWVAEIRNKAYNHATYYAESVKGRFTISRDDSNSRVSLQMNNLRAEDTGVYYCTAGDSSHSYWQGTLVTVSA

SEQ ID NO: 98 - protein, 107 aa, synthetic construct
  REGION 1..107: note = Amino acid sequence of light chain variable region of monoclonal antibody 180C 7G8
DIVMTQSQKFMSTSVGDRVSIICKASQNVRTAVAWYQQKPGQSPKTLIYLASNRHTGVPDRFTGSGSGTDFTLTISNVQAEDLADYFCLQHWNYPLTFGGGTKLEIK

SEQ ID NO: 99 - DNA, 351 bp, synthetic construct
  misc_feature 1..351: note = DNA sequence of heavy chain variable region of monoclonal antibody 180C 7G8
  source 1..351: mol_type = other DNA
gaagtgaaacttgaggagtctggaggaggcttggtgcaacctggaggatccgtgaaactctcttgtgctgttttggattcactttcagtgatccctggatggactgggtccgccagtcgccagagaaggggcttgagtgggttgctgaaattagaaacaaagcttataatcatgccacatattatgctgagtctgttaagggaggttcaccatctcaagagatgattccaacagtagagtgtccctgcaaatgaacaacttaagagctgaagacactggcgtttattactgtaccgccggtgactccagtcattcttattggggcaagggactctggtcactgtctctgca

SEQ ID NO: 100 - DNA, 319 bp, synthetic construct
  misc_feature 1..319: note = DNA sequence of light chain variable region of monoclonal antibody 180C 7G8
  source 1..319: mol_type = other DNA
gacattgtgatgacccagtctcaaaaattcatgtccacatcagtaggagacagggtcagcatcatctcaaggccagtcagaatgttcgtactgctgtagcctggtatcaacagaaaccagggcagtctcctaaaacactgatttacttggcatccaaccggcacactggagtccctgatcgcttcacaggcagtgatctgggacagatttcactctcaccattagcaatgtacaagctgaagacctggcagattatttctgtctgcaacattggaattatcctctcacgttcggaggggggaccaaactggaaataaaa

SEQ ID NO: 101 - protein, 134 aa, synthetic construct
  REGION 1..134: note = Amino acid sequence of heavy chain of monoclonal antibody 180C 8A10
MGWSYIFLFLVATVTDVHSQVQLQQPGAELVKPGASVKLSCKASGYTFTNYWINWVKQRPGQGLEWLGEINPSNSNTKYNEKFKSRATLTADKSSSTAYMQLHSLTSEDSAVYYCNYYDGGHDSWGQTTLTVSS

SEQ ID NO: 102 - protein, 5 aa, synthetic construct
  REGION 1..5: note = Amino acid sequence of heavy chain CDR1 of monoclonal antibody 180C 8A10
NYWIN

SEQ ID NO: 103 - protein, 17 aa, synthetic construct
  REGION 1..17: note = Amino acid sequence of heavy chain CDR2 of monoclonal antibody 180C 8A10
EINPSNSNTKYNEKFKS

SEQ ID NO: 104 - protein, 7 aa, synthetic construct
  REGION 1..7: note = Amino acid sequence of heavy chain CDR3 of monoclonal antibody 180C 8A10
YDGGHDS

SEQ ID NO: 105 - protein, 127 aa, synthetic construct
  REGION 1..127: note = Amino acid sequence of light chain of monoclonal antibody 180C 8A10
MEFHTQVFVFVFLWLSGVDGDIVMTQSQKFMSTTLGDRVSIICKASQNVGTAVAWYQQKPGQSPKLLIYSTSNRYTGVPDRFTGSGSGTDFSLTISSVQSEDLADYFCHQYSSYLYTFGGGTRLEIK

SEQ ID NO: 106 - protein, 11 aa, synthetic construct
  REGION 1..11: note = Amino acid sequence of light chain CDR1 of monoclonal antibody 180C 8A10
KASQNVGTAVA

SEQ ID NO: 107 - protein, 7 aa, synthetic construct
  REGION 1..7: note = Amino acid sequence of light chain CDR2 of monoclonal antibody 180C 8A10
STSNRYT

SEQ ID NO: 108 - protein, 9 aa, synthetic construct
  REGION 1..9: note = Amino acid sequence of light chain CDR3 of monoclonal antibody 180C 8A10
HQYSSYLYT

SEQ ID NO: 109 - DNA, 402 bp, synthetic construct
  misc_feature 1..402: note = DNA sequence of heavy chain of monoclonal antibody 180C 8A10
  source 1..402: mol_type = other DNA
atgggatggagctatatcttcctctttttggtagcaacagttacagatgtccactcccaggtccaactgcagcagcctggggctgagcttgtgaagcctggggcttcagtgaagttgtcctgcaaggttctggctacaccttcaccaactactggataaactgggtgaaacagaggcctgggcaaggccttgagtggcttggagaaattaatcctagcaatagtaatactaaatataatgagaaattcaaaagcgggccacactgactgcagacaaatcctccagcacagcctacatgcaactccacagcctaacctctgaggactctgcggtctactactgtaattattacgatggtggccacgactcctggggccagggaccactctcacagtctcctca

SEQ ID NO: 110 - DNA, 15 bp, synthetic construct
  misc_feature 1..15: note = DNA sequence of heavy chain CDR1 of monoclonal antibody 180C 8A10
  source 1..15: mol_type = other DNA
aactactggataaac

SEQ ID NO: 111 - DNA, 51 bp, synthetic construct
  misc_feature 1..51: note = DNA sequence of heavy chain CDR2 of monoclonal antibody 180C 8A10
  source 1..51: mol_type = other DNA
gaaattaatcctagcaatagtaatactaaatataatgagaaattcaaaagc

SEQ ID NO: 112 - DNA, 21 bp, synthetic construct
  misc_feature 1..21: note = DNA sequence of heavy chain CDR3 of monoclonal antibody 180C 8A10
  source 1..21: mol_type = other DNA
tacgatggtggccacgactcc

SEQ ID NO: 113 - DNA, 379 bp, synthetic construct
  misc_feature 1..379: note = DNA sequence of light chain of monoclonal antibody 180C 8A10
  source 1..379: mol_type = other DNA
atggagtttcatactcaggtctttgtattcgtgtttctctggttgtctggtgttgatggagacattgtgatgacccagtctcaaaaattcatgtccacaacattaggagacagggtcagcatcatctcaaggccagtcagaatgtgggtactgctgtcgcctggtatcaacagaaaccaggacaatctcctaaactactgatttactcaacatccaatcgctacactggagtccctgatcgcttcacaggcagtgatctgggacagatttcagtctcaccattagcagtgtgcagtctgaggacctggcagattatttctgtcaccaatatagcagctatctatacacgttcgggggggggaccaggctggaaattaaa

SEQ ID NO: 114 - DNA, 33 bp, synthetic construct
  misc_feature 1..33: note = DNA sequence of light chain CDR1 of monoclonal antibody 180C 8A10
  source 1..33: mol_type = other DNA
aaggccagtcagaatgtgggtactgctgtcgcc

SEQ ID NO: 115 - DNA, 21 bp, synthetic construct
  misc_feature 1..21: note = DNA sequence of light chain CDR2 of monoclonal antibody 180C 8A10
  source 1..21: mol_type = other DNA
tcaacatccaatcgctacact

SEQ ID NO: 116 - DNA, 27 bp, synthetic construct
  misc_feature 1..27: note = DNA sequence of light chain CDR3 of monoclonal antibody 180C 8A10
  source 1..27: mol_type = other DNA
caccaatatagcagctatctatacacg

SEQ ID NO: 117 - protein, 115 aa, synthetic construct
  REGION 1..115: note = Amino acid sequence of heavy chain variable region of monoclonal antibody 180C 8A10
QVQLQQPGAELVKPGASVKLSCKASGYTFTNYWINWVKQRPGQGLEWLGEINPSNSNTKYNEKFKSRATLTADKSSSTAYMQLHSLTSEDSAVYYCNYYDGGHDSWGQTTLTVSS

SEQ ID NO: 118 - protein, 107 aa, synthetic construct
  REGION 1..107: note = Amino acid sequence of light chain variable region of monoclonal antibody 180C 8A10
DIVMTQSQKFMSTTLGDRVSIICKASQNVGTAVAWYQQKPGQSPKLLIYSTSNRYTGVPDRFTGSGSGTDFSLTISSVQSEDLADYFCHQYSSYLYTFGGGTRLEIK

SEQ ID NO: 119 - DNA, 345 bp, synthetic construct
  misc_feature 1..345: note = DNA sequence of heavy chain variable region of monoclonal antibody 180C 8A10
  source 1..345: mol_type = other DNA
caggtccaactgcagcagcctggggctgagcttgtgaagcctggggcttcagtgaagttgtcctgcaaggttctggctacaccttcaccaactactggataaactgggtgaaacagaggcctgggcaaggccttgagtggcttggagaaattaatcctagcaatagtaatactaaatataatgagaaattcaaaagcgggccacactgactgcagacaaatcctccagcacagcctacatgcaactccacagcctaacctctgaggactctgcggtctactactgtaattattacgatggtggccacgactcctggggccagggaccactctcacagtctcctca

SEQ ID NO: 120 - DNA, 319 bp, synthetic construct
  misc_feature 1..319: note = DNA sequence of light chain variable region of monoclonal antibody 180C 8A10
  source 1..319: mol_type = other DNA
gacattgtgatgacccagtctcaaaaattcatgtccacaacattaggagacagggtcagcatcatctcaaggccagtcagaatgtgggtactgctgtcgcctggtatcaacagaaaccaggacaatctcctaaactactgatttactcaacatccaatcgctacactggagtccctgatcgcttcacaggcagtgatctgggacagatttcagtctcaccattagcagtgtgcagtctgaggacctggcagattatttctgtcaccaatatagcagctatctatacacgttcgggggggggaccaggctggaaattaaa

SEQ ID NO: 121 - protein, 133 aa, synthetic construct
  REGION 1..133: note = Amino acid sequence for heavy chain of monoclonal antibody 180C 9D5
MGWNYIFLFLIATVTDVHSQVRLQQPGTDLVKPGASVKLSCRASGFTFTNYWMNWVKQRPGQGLEWIGEINTNGGTKYNENFKNKATLTVDQSSSTAYIQLSGLTSEDSAVYFCNYFDGNYDYWGQGTLTVSS

SEQ ID NO: 122 - protein, 5 aa, synthetic construct
  REGION 1..5: note = Amino acid sequence for heavy chain CDR1 of monoclonal antibody 180C 9D5
NYWMN

SEQ ID NO: 123 - protein, 16 aa, synthetic construct
  REGION 1..16: note = Amino acid sequence for heavy chain CDR2 of monoclonal antibody 180C 9D5
EINTNGGTKYNENFKN

SEQ ID NO: 124 - protein, 7 aa, synthetic construct
  REGION 1..7: note = Amino acid sequence for heavy chain CDR3 of monoclonal antibody 180C 9D5
FDGNYDY

SEQ ID NO: 125 - protein, 127 aa, synthetic construct
  REGION 1..127: note = Amino acid sequence for light chain of monoclonal antibody 180C 9D5
MEFHTQVFVFVFLWLSGVDGDIVMTQSQKFMSTPVGDRVSLTCRASQNVGTAVAWYQQKPGQPPKLLIYSTSNRFTGVPSRFTGSGSGTDFTLTITNMQSEDLADYFCHQYSSYPYTFGGGTKLEIK

SEQ ID NO: 126 - protein, 11 aa, synthetic construct
  REGION 1..11: note = Amino acid sequence for light chain CDR1 of monoclonal antibody 180C 9D5
RASQNVGTAVA

SEQ ID NO: 127 - protein, 7 aa, synthetic construct
  REGION 1..7: note = Amino acid sequence for light chain CDR2 of monoclonal antibody 180C 9D5
STSNRFT

SEQ ID NO: 128 - protein, 9 aa, synthetic construct
  REGION 1..9: note = Amino acid sequence for light chain CDR3 of monoclonal antibody 180C 9D5
HQYSSYPYT

SEQ ID NO: 129 - DNA, 399 bp, synthetic construct
  misc_feature 1..399: note = DNA sequence for heavy chain of monoclonal antibody 180C 9D5
  source 1..399: mol_type = other DNA
atgggatggaactatatcttcctctttttgatagcaacagttacagatgtccactcccaggtccgactgcagcagcctgggactgatcttgtgaagcctggggcttcagtgaagctgtcctgcagggttctggcttcacattcaccaattattggatgaattgggtgaaacagaggcctggacaaggccttgagtggattggagagattaataccaacggtggtactaagtacaatgagaatttcaagaataagccacactgactgtggaccaatcctccagcacagcctacattcaactcagcggcctgacatctgaggactctgcggtctatttctgtaattacttcgatggtaactacgactactggggccaaggcacactctcacagtctcctca

SEQ ID NO: 130 - DNA, 15 bp, synthetic construct
  misc_feature 1..15: note = DNA sequence for heavy chain CDR1 of monoclonal antibody 180C 9D5
  source 1..15: mol_type = other DNA
aattattggatgaat

SEQ ID NO: 131 - DNA, 48 bp, synthetic construct
  misc_feature 1..48: note = DNA sequence for heavy chain CDR2 of monoclonal antibody 180C 9D5
  source 1..48: mol_type = other DNA
gagattaataccaacggtggtactaagtacaatgagaatttcaagaat

SEQ ID NO: 132 - DNA, 21 bp, synthetic construct
  misc_feature 1..21: note = DNA sequence for heavy chain CDR3 of monoclonal antibody 180C 9D5
  source 1..21: mol_type = other DNA
ttcgatggtaactacgactac

SEQ ID NO: 133 - DNA, 379 bp, synthetic construct
  misc_feature 1..379: note = DNA sequence for light chain of monoclonal antibody 180C 9D5
  source 1..379: mol_type = other DNA
atggagtttcatactcaggtctttgtattcgtgtttctctggttgtctggtgttgatggagacattgtgatgacccagtctcaaaaattcatgtccacaccagtgggagacagggtcagcctcacctcagggccagtcagaatgtgggtactgctgtagcctggtatcaacagaaaccaggacaacctcctaaattactaatttattcaacatccaatcgattcactggagtccctagtcgcttcacaggcagtgatctgggacagattttactctcaccattaccaatatgcagtctgaagacctggcagattatttctgtcaccaatatagcagctatccgtacacattcggaggggggaccaaactggaaataaaa

SEQ ID NO: 134 - DNA, 33 bp, synthetic construct
  misc_feature 1..33: note = DNA sequence for light chain CDR1 of monoclonal antibody 180C 9D5
  source 1..33: mol_type = other DNA
agggccagtcagaatgtgggtactgctgtagcc

SEQ ID NO: 135 - DNA, 21 bp, synthetic construct
  misc_feature 1..21: note = DNA sequence for light chain CDR2of monoclonal antibody 180C 9D5
  source 1..21: mol_type = other DNA
tcaacatccaatcgattcact

SEQ ID NO: 136 - DNA, 27 bp, synthetic construct
  misc_feature 1..27: note = DNA sequence for light chain CDR3 of monoclonal antibody 180C 9D5
  source 1..27: mol_type = other DNA
caccaatatagcagctatccgtacaca

SEQ ID NO: 137 - protein, 114 aa, synthetic construct
  REGION 1..114: note = Amino acid sequence for heavy chain variable region of monoclonal antibody 180C 9D5
QVRLQQPGTDLVKPGASVKLSCRASGFTFTNYWMNWVKQRPGQGLEWIGEINTNGGTKYNENFKNKATLTVDQSSSTAYIQLSGLTSEDSAVYFCNYFDGNYDYWGQGTLTVSS

SEQ ID NO: 138 - protein, 107 aa, synthetic construct
  REGION 1..107: note = Amino acid sequence for light chain variable region of monoclonal antibody 180C 9D5
DIVMTQSQKFMSTPVGDRVSLTCRASQNVGTAVAWYQQKPGQPPKLLIYSTSNRFTGVPSRFTGSGSGTDFTLTITNMQSEDLADYFCHQYSSYPYTFGGGTKLEIK

SEQ ID NO: 139 - DNA, 342 bp, synthetic construct
  misc_feature 1..342: note = DNA sequence for heavy chain variable region of monoclonal antibody 180C 9D5
  source 1..342: mol_type = other DNA
caggtccgactgcagcagcctgggactgatcttgtgaagcctggggcttcagtgaagctgtcctgcagggttctggcttcacattcaccaattattggatgaattgggtgaaacagaggcctggacaaggccttgagtggattggagagattaataccaacggtggtactaagtacaatgagaatttcaagaataagccacactgactgtggaccaatcctccagcacagcctacattcaactcagcggcctgacatctgaggactctgcggtctatttctgtaattacttcgatggtaactacgactactggggccaaggcacactctcacagtctcctca

SEQ ID NO: 140 - DNA, 319 bp, synthetic construct
  misc_feature 1..319: note = DNA sequence for light chain variable region of monoclonal antibody 180C 9D5
  source 1..319: mol_type = other DNA
gacattgtgatgacccagtctcaaaaattcatgtccacaccagtgggagacagggtcagcctcacctcagggccagtcagaatgtgggtactgctgtagcctggtatcaacagaaaccaggacaacctcctaaattactaatttattcaacatccaatcgattcactggagtccctagtcgcttcacaggcagtgatctgggacagattttactctcaccattaccaatatgcagtctgaagacctggcagattatttctgtcaccaatatagcagctatccgtacacattcggaggggggaccaaactggaaataaaa

SEQ ID NO: 141 - protein, 137 aa, synthetic construct
  REGION 1..137: note = Amino acid sequence of heavy chain of monoclonal antibody 180C 9G8
MYLGLNCVIIVFLLKGVQSEVKLEESGGGLVQPGGSMKLSCAASGFTFSDAWMDWVRQSPEKGLEWVAEIRNKANNHATYYAESVKGRFTISRDDSNSRMFLQMDSLRAEDTGIYYCIGGIPRYFDVGAGTTVTVSS

SEQ ID NO: 142 - protein, 5 aa, synthetic construct
  REGION 1..5: note = Amino acid sequence of heavy chain CDR1 of monoclonal antibody 180C 9G8
DAWMD

SEQ ID NO: 143 - protein, 19 aa, synthetic construct
  REGION 1..19: note = Amino acid sequence of heavy chain CDR2 of monoclonal antibody 180C 9G8
EIRNKANNHATYYAESVKG

SEQ ID NO: 144 - protein, 8 aa, synthetic construct
  REGION 1..8: note = Amino acid sequence of heavy chain CDR3 of monoclonal antibody 180C 9G8
GIPRYFDV

SEQ ID NO: 145 - protein, 127 aa, synthetic construct
  REGION 1..127: note = Amino acid sequence of light chain of monoclonal antibody 180C 9G8
MESQTQVFVYMLLWLSGVDGDIVMTQSQKFMSTSVGDRVSITCKASQNVRTYVAWYQQKPGQSPKALLYSASYRYSGVPDRFIGSGSGTQFTLTISDVQSEDLAEYFCHQYNSYPLTFGGGTKLEIQ

SEQ ID NO: 146 - protein, 11 aa, synthetic construct
  REGION 1..11: note = Amino acid sequence of light chain CDR1 of monoclonal antibody 180C 9G8
KASQNVRTYVA

SEQ ID NO: 147 - protein, 7 aa, synthetic construct
  REGION 1..7: note = Amino acid sequence of light chain CDR2 of monoclonal antibody 180C 9G8
SASYRYS

SEQ ID NO: 148 - protein, 9 aa, synthetic construct
  REGION 1..9: note = Amino acid sequence of light chain CDR3 of monoclonal antibody 180C 9G8
HQYNSYPLT

SEQ ID NO: 149 - DNA, 411 bp, synthetic construct
  misc_feature 1..411: note = DNA sequence of heavy chain of monoclonal antibody 180C 9G8
  source 1..411: mol_type = other DNA
atgtacttgggactgaactgtgtaatcatagtttttctcttaaaaggtgtccagagtgaagtgaagcttgaggagtctggaggaggcttggtgcaacctggaggatccatgaaactctcttgtgctgttctggattcacttttagtgatgcctggatggactgggtccgccagtctccagagaaggggcttgagtgggttgctgaaattagaaacaaagctaataatcatgcaacatattatgctgagtctgtgaggggaggttcaccatctcaagagatgattccaacagtagaatgttcctgcaaatggacagtttaagagctgaagacactggcatttattactgtatcgggggaattccacgctacttcgatgtctgggcgcagggaccacggtcaccgtctcctca

SEQ ID NO: 150 - DNA, 15 bp, synthetic construct
  misc_feature 1..15: note = DNA sequence of heavy chain CDR1 of monoclonal antibody 180C 9G8
  source 1..15: mol_type = other DNA
gatgcctggatggac

SEQ ID NO: 151 - DNA, 56 bp, synthetic construct
  misc_feature 1..56: note = DNA sequence of heavy chain CDR2 of monoclonal antibody 180C 9G8
  source 1..56: mol_type = other DNA
gaaattagaaacaaagctaataatcatgcaacatattatgctgagtctgtgagggg

SEQ ID NO: 152 - DNA, 24 bp, synthetic construct
  misc_feature 1..24: note = DNA sequence of heavy chain CDR3 of monoclonal antibody 180C 9G8
  source 1..24: mol_type = other DNA
ggaattccacgctacttcgatgtc

SEQ ID NO: 153 - DNA, 379 bp, synthetic construct
  misc_feature 1..379: note = DNA sequence of light chain of monoclonal antibody 180C 9G8
  source 1..379: mol_type = other DNA
atggagtcacagactcaggtctttgtatacatgttgctgtggttgtctggtgttgatggagacattgtgatgacccagtctcaaaaattcatgtccacatcagtcggagacagggtcagcattacctcaaggccagtcagaatgtgagaacttatgttgcctggtatcaacagaaaccaggacaatcccctaaagcactgctttattcggcttcttaccgatacagtggagtccctgatcgcttcataggcagtgatctgggacacaattcactctcaccatcagcgatgtgcagtctgaagacttggcagaatatttctgtcaccaatataacagctatcctctaacgttcggaggggggaccaaactggaaatacaa

SEQ ID NO: 154 - DNA, 33 bp, synthetic construct
  misc_feature 1..33: note = DNA sequence of light chain CDR1 of monoclonal antibody 180C 9G8
  source 1..33: mol_type = other DNA
aaggccagtcagaatgtgagaacttatgttgcc

SEQ ID NO: 155 - DNA, 21 bp, synthetic construct
  misc_feature 1..21: note = DNA sequence of light chain CDR2 of monoclonal antibody 180C 9G8
  source 1..21: mol_type = other DNA
tcggcttcttaccgatacagt

SEQ ID NO: 156 - DNA, 27 bp, synthetic construct
  misc_feature 1..27: note = DNA sequence of light chain CDR3 of monoclonal antibody 180C 9G8
  source 1..27: mol_type = other DNA
caccaatataacagctatcctctaacg

SEQ ID NO: 157 - protein, 118 aa, synthetic construct
  REGION 1..118: note = Amino acid sequence of heavy chain variable region of monoclonal antibody 180C 9G8
EVKLEESGGGLVQPGGSMKLSCAASGFTFSDAWMDWVRQSPEKGLEWVAEIRNKANNHATYYAESVKGRFTISRDDSNSRMFLQMDSLRAEDTGIYYCIGGIPRYFDVGAGTTVTVSS

SEQ ID NO: 158 - protein, 107 aa, synthetic construct
  REGION 1..107: note = Amino acid sequence of light chain variable region of monoclonal antibody 180C 9G8
DIVMTQSQKFMSTSVGDRVSITCKASQNVRTYVAWYQQKPGQSPKALLYSASYRYSGVPDRFIGSGSGTQFTLTISDVQSEDLAEYFCHQYNSYPLTFGGGTKLEIQ

SEQ ID NO: 159 - DNA, 354 bp, synthetic construct
  misc_feature 1..354: note = DNA sequence of heavy chain variable region of monoclonal antibody 180C 9G8
  source 1..354: mol_type = other DNA
gaagtgaagcttgaggagtctggaggaggcttggtgcaacctggaggatccatgaaactctcttgtgctgttctggattcacttttagtgatgcctggatggactgggtccgccagtctccagagaaggggcttgagtgggttgctgaaattagaaacaaagctaataatcatgcaacatattatgctgagtctgtgaggggaggttcaccatctcaagagatgattccaacagtagaatgttcctgcaaatggacagtttaagagctgaagacactggcatttattactgtatcgggggaattccacgctacttcgatgtctgggcgcagggaccacggtcaccgtctcctca

SEQ ID NO: 160 - DNA, 319 bp, synthetic construct
  misc_feature 1..319: note = DNA sequence of light chain variable region of monoclonal antibody 180C 9G8
  source 1..319: mol_type = other DNA
gacattgtgatgacccagtctcaaaaattcatgtccacatcagtcggagacagggtcagcattacctcaaggccagtcagaatgtgagaacttatgttgcctggtatcaacagaaaccaggacaatcccctaaagcactgctttattcggcttcttaccgatacagtggagtccctgatcgcttcataggcagtgatctgggacacaattcactctcaccatcagcgatgtgcagtctgaagacttggcagaatatttctgtcaccaatataacagctatcctctaacgttcggaggggggaccaaactggaaatacaa

SEQ ID NO: 161 - protein, 136 aa, synthetic construct
  REGION 1..136: note = Amino acid sequence of heavy chain of monoclonal antibody 179B 9G1.1
MEWPCIFLFLLSVTEGVHSQVQLQQSGPELVKPGASVMISCKVFGDIFSSSWMNWVKQRPGKGLEWIGWICPGNGATKYNGKFKGKATLTADKSSGTGYMQLSSLTSEDSAVYFCELGRRDGYLDVWAGTTVTVSS

SEQ ID NO: 162 - protein, 5 aa, synthetic construct
  REGION 1..5: note = Amino acid sequence of heavy chain CDR1 of monoclonal antibody 179B 9G1.1
SSWMN

SEQ ID NO: 163 - protein, 17 aa, synthetic construct
  REGION 1..17: note = Amino acid sequence of heavy chain CDR2 of monoclonal antibody 179B 9G1.1
WICPGNGATKYNGKFKG

SEQ ID NO: 164 - protein, 9 aa, synthetic construct
  REGION 1..9: note = Amino acid sequence of heavy chain CDR3 of monoclonal antibody 179B 9G1.1
GRRDGYLDV

SEQ ID NO: 165 - protein, 127 aa, synthetic construct
  REGION 1..127: note = Amino acid sequence of light chain of monoclonal antibody 179B 9G1.1
MESQTQVFVYMLLWLSGVDGDIVMTQSQKFIFTSEGDRVSVTCKASQNVGTNIAWYQQKPGQSPKALIYSASNRYSGVPDRFTGSGSGTDFTLTFSNVQSEDLAEYFCLQYNNYPWTFGGGTKLEIK

SEQ ID NO: 166 - protein, 11 aa, synthetic construct
  REGION 1..11: note = Amino acid sequence of light chain CDR1 of monoclonal antibody 179B 9G1.1
KASQNVGTNIA

SEQ ID NO: 167 - protein, 7 aa, synthetic construct
  REGION 1..7: note = Amino acid sequence of light chain CDR2 of monoclonal antibody 179B 9G1.1
SASNRYS

SEQ ID NO: 168 - protein, 9 aa, synthetic construct
  REGION 1..9: note = Amino acid sequence of light chain CDR3 of monoclonal antibody 179B 9G1.1
LQYNNYPWT

SEQ ID NO: 169 - DNA, 408 bp, synthetic construct
  misc_feature 1..408: note = DNA sequence of heavy chain of monoclonal antibody 179B 9G1.1
  source 1..408: mol_type = other DNA
atggaatggccttgtatctttctcttcctcctgtcagtaactgaaggtgtccactcccaggtccagttgcagcagtctggacctgaactggtgaagcctggggcctcagtgatgatttcctgcaaagttttggcgatatattcagtagttcctggatgaactgggtgaagcagcggcctggaaagggtcttgagtggattggatggatttgtcctggaaatggtgccactaagtacaatggaaagttcaagggcaggccacactgactgcagataaatcctcaggcacaggctacatgcagctcagcagcctgacatctgaggactctgcggtctatttctgtgaactgggacgtcgggacggttatctcgatgtctgggggcagggacaacggtcaccgtctcctca

SEQ ID NO: 170 - DNA, 15 bp, synthetic construct
  misc_feature 1..15: note = DNA sequence of heavy chain CDR1 of monoclonal antibody 179B 9G1.1
  source 1..15: mol_type = other DNA
agttcctggatgaac

SEQ ID NO: 171 - DNA, 51 bp, synthetic construct
  misc_feature 1..51: note = DNA sequence of heavy chain CDR2 of monoclonal antibody 179B 9G1.1
  source 1..51: mol_type = other DNA
tggatttgtcctggaaatggtgccactaagtacaatggaaagttcaagggc

SEQ ID NO: 172 - DNA, 27 bp, synthetic construct
  misc_feature 1..27: note = DNA sequence of heavy chain CDR3 of monoclonal antibody 179B 9G1.1
  source 1..27: mol_type = other DNA
ggacgtcgggacggttatctcgatgtc

SEQ ID NO: 173 - DNA, 379 bp, synthetic construct
  misc_feature 1..379: note = DNA sequence of light chain of monoclonal antibody 179B 9G1.1
  source 1..379: mol_type = other DNA
atggagtcacagactcaggtctttgtatacatgttgctgtggttgtctggtgttgatggagacattgtgatgacacagtctcaaaaattcatattcacatcagaaggtgacagggtcagcgtcacctcaaggccagtcagaatgtgggtactaatattgcctggtatcaacagaaaccaggacaatctcctaaagcactgatttactcggcatccaaccggtacagtggagtccctgatcgcttcacaggcagtgatctgggacagatttcactctcactttcagcaatgtgcagtctgaagacttggcagaatatttctgtctgcaatataacaactatccgtggacgttcggtggaggcaccaagctggaaatcaaa

SEQ ID NO: 174 - DNA, 33 bp, synthetic construct
  misc_feature 1..33: note = DNA sequence of light chain CDR1 of monoclonal antibody 179B 9G1.1
  source 1..33: mol_type = other DNA
aaggccagtcagaatgtgggtactaatattgcc

SEQ ID NO: 175 - DNA, 21 bp, synthetic construct
  misc_feature 1..21: note = DNA sequence of light chain CDR2 of monoclonal antibody 179B 9G1.1
  source 1..21: mol_type = other DNA
tcggcatccaaccggtacagt

SEQ ID NO: 176 - DNA, 27 bp, synthetic construct
  misc_feature 1..27: note = DNA sequence of light chain CDR3 of monoclonal antibody 179B 9G1.1
  source 1..27: mol_type = other DNA
ctgcaatataacaactatccgtggacg

SEQ ID NO: 177 - protein, 117 aa, synthetic construct
  REGION 1..117: note = Amino acid sequence of heavy chain variable region of monoclonal antibody 179B 9G1.1
QVQLQQSGPELVKPGASVMISCKVFGDIFSSSWMNWVKQRPGKGLEWIGWICPGNGATKYNGKFKGKATLTADKSSGTGYMQLSSLTSEDSAVYFCELGRRDGYLDVWAGTTVTVSS

SEQ ID NO: 178 - protein, 107 aa, synthetic construct
  REGION 1..107: note = Amino acid sequence of light chain variable region of monoclonal antibody 179B 9G1.1
DIVMTQSQKFIFTSEGDRVSVTCKASQNVGTNIAWYQQKPGQSPKALIYSASNRYSGVPDRFTGSGSGTDFTLTFSNVQSEDLAEYFCLQYNNYPWTFGGGTKLEIK

SEQ ID NO: 179 - DNA, 351 bp, synthetic construct
  misc_feature 1..351: note = DNA sequence of heavy chain variable region of monoclonal antibody 179B 9G1.1
  source 1..351: mol_type = other DNA
caggtccagttgcagcagtctggacctgaactggtgaagcctggggcctcagtgatgatttcctgcaaagttttggcgatatattcagtagttcctggatgaactgggtgaagcagcggcctggaaagggtcttgagtggattggatggatttgtcctggaaatggtgccactaagtacaatggaaagttcaagggcaggccacactgactgcagataaatcctcaggcacaggctacatgcagctcagcagcctgacatctgaggactctgcggtctatttctgtgaactgggacgtcgggacggttatctcgatgtctgggggcagggacaacggtcaccgtctcctca

SEQ ID NO: 180 - DNA, 319 bp, synthetic construct
  misc_feature 1..319: note = DNA sequence of light chain variable region of monoclonal antibody 179B 9G1.1
  source 1..319: mol_type = other DNA
gacattgtgatgacacagtctcaaaaattcatattcacatcagaaggtgacagggtcagcgtcacctcaaggccagtcagaatgtgggtactaatattgcctggtatcaacagaaaccaggacaatctcctaaagcactgatttactcggcatccaaccggtacagtggagtccctgatcgcttcacaggcagtgatctgggacagatttcactctcactttcagcaatgtgcagtctgaagacttggcagaatatttctgtctgcaatataacaactatccgtggacgttcggtggaggcaccaagctggaaatcaaa

SEQ ID NO: 181 - protein, 138 aa, synthetic construct
  REGION 1..138: note = Amino acid sequence of heavy chain of monoclonal antibody 179B 9E12
MGWSWIFLFLLSGTAGVHCQIQLQQSGPELVKPGASVKISCKASDYSFTNSYINWVKQRPGQGLEWIGWIYPGSTNTKYNEKFWDKATLTVDTSSNTAYMELRGLTSEDSAVYFCEMGTHYSYDVMDWGQGTSVTVSS

SEQ ID NO: 182 - protein, 5 aa, synthetic construct
  REGION 1..5: note = Amino acid sequence of heavy chain CDR1 of monoclonal antibody 179B 9E12
NSYIN

SEQ ID NO: 183 - protein, 17 aa, synthetic construct
  REGION 1..17: note = Amino acid sequence of heavy chain CDR2 of monoclonal antibody 179B 9E12
WIYPGSTNTKYNEKFWD

SEQ ID NO: 184 - protein, 10 aa, synthetic construct
  REGION 1..10: note = Amino acid sequence of heavy chain CDR3 of monoclonal antibody 179B 9E12
GTHYSYDVMD

SEQ ID NO: 185 - protein, 127 aa, synthetic construct
  REGION 1..127: note = Amino acid sequence of light chain of monoclonal antibody 179B 9E12
MEFQTQVFVFVLLWLSGVDGDIVMTPSQKFMSTSVGDRVSITCKASQNVRTSVGWYQQKPGQSPKPLIYATSNRHTGVPDRFTGSGSGTDFSLTITNVQSEDQADYFCLQHWNYPWTFGGGTKLEIK

SEQ ID NO: 186 - protein, 11 aa, synthetic construct
  REGION 1..11: note = Amino acid sequence of light chain CDR1 of monoclonal antibody 179B 9E12
KASQNVRTSVG

SEQ ID NO: 187 - protein, 7 aa, synthetic construct
  REGION 1..7: note = Amino acid sequence of light chain CDR2 of monoclonal antibody 179B 9E12
ATSNRHT

SEQ ID NO: 188 - protein, 9 aa, synthetic construct
  REGION 1..9: note = Amino acid sequence of light chain CDR3 of monoclonal antibody 179B 9E12
LQHWNYPWT

SEQ ID NO: 189 - DNA, 414 bp, synthetic construct
  misc_feature 1..414: note = DNA sequence of heavy chain of monoclonal antibody 179 9E12
  source 1..414: mol_type = other DNA
atgggatggagctggatctttcttttcctcctgtcaggaactgcaggtgtccattgccagatccagctgcagcagtctggacctgagttggtgaagcctggggcttcagtgaagatatcctgcaaggttctgactacagcttcactaactcctatataaactgggtgaagcagaggcctggacagggacttgagtggattggatggatttatcctggaagcactaatactaagtataatgagaagttctgggacaggccacattgactgtggacacatcctccaacacagcctacatggagctcaggggtctgacctctgaggactctgcggtctatttctgtgaaatggggactcactatagttacgatgttatggactatggggtcaaggaacctcagtcaccgtctcctca

SEQ ID NO: 190 - DNA, 15 bp, synthetic construct
  misc_feature 1..15: note = DNA sequence of heavy chain CDR1 of monoclonal antibody 179 9E12
  source 1..15: mol_type = other DNA
aactcctatataaac

SEQ ID NO: 191 - DNA, 51 bp, synthetic construct
  misc_feature 1..51: note = DNA sequence of heavy chain CDR2 of monoclonal antibody 179 9E12
  source 1..51: mol_type = other DNA
tggatttatcctggaagcactaatactaagtataatgagaagttctgggac

SEQ ID NO: 192 - DNA, 33 bp, synthetic construct
  misc_feature 1..33: note = DNA sequence of heavy chain CDR3 of monoclonal antibody 179 9E12
  source 1..33: mol_type = other DNA
gggactcactatagttacgatgttatggactac

SEQ ID NO: 193 - DNA, 379 bp, synthetic construct
  misc_feature 1..379: note = DNA sequence of light chain of monoclonal antibody 179 9E12
  source 1..379: mol_type = other DNA
atggagtttcagacccaggtctttgtattcgtgttgctctggttgtctggtgttgatggagacattgtgatgaccccgtctcaaaaattcatgtccacatcagtaggagacagggtcagcatcacctcaaggccagtcagaatgttcgtacttctgtaggctggtatcaacagaaaccagggcagtctcctaaaccactgatttacgcgacatccaaccggcacactggagtccctgatcgcttcacaggcagtgatctgggacagatttcagtctcaccattaccaatgtgcaatctgaagaccaggcagattatttctgtctgcagcattggaattatccgtggacgttcggtggaggcaccaagctggaaatcaaa

SEQ ID NO: 194 - DNA, 33 bp, synthetic construct
  misc_feature 1..33: note = DNA sequence of light chain CDR1 of monoclonal antibody 179 9E12
  source 1..33: mol_type = other DNA
aaggccagtcagaatgttcgtacttctgtaggc

SEQ ID NO: 195 - DNA, 21 bp, synthetic construct
  misc_feature 1..21: note = DNA sequence of light chain CDR2 of monoclonal antibody 179 9E12
  source 1..21: mol_type = other DNA
gcgacatccaaccggcacact

SEQ ID NO: 196 - DNA, 27 bp, synthetic construct
  misc_feature 1..27: note = DNA sequence of light chain CDR3 of monoclonal antibody 179 9E12
  source 1..27: mol_type = other DNA
ctgcagcattggaattatccgtggacg

SEQ ID NO: 197 - protein, 119 aa, synthetic construct
  REGION 1..119: note = Amino acid sequence of heavy chain variable region of monoclonal antibody 179 9E12
QIQLQQSGPELVKPGASVKISCKASDYSFTNSYINWVKQRPGQGLEWIGWIYPGSTNTKYNEKFWDKATLTVDTSSNTAYMELRGLTSEDSAVYFCEMGTHYSYDVMDWGQGTSVTVSS

SEQ ID NO: 198 - protein, 107 aa, synthetic construct
  REGION 1..107: note = Amino acid sequence of light chain variable region of monoclonal antibody 179 9E12
DIVMTPSQKFMSTSVGDRVSITCKASQNVRTSVGWYQQKPGQSPKPLIYATSNRHTGVPDRFTGSGSGTDFSLTITNVQSEDQADYFCLQHWNYPWTFGGGTKLEIK

SEQ ID NO: 199 - DNA, 357 bp, synthetic construct
  misc_feature 1..357: note = DNA sequence of heavy chain variable region of monoclonal antibody 179 9E12
  source 1..357: mol_type = other DNA
cagatccagctgcagcagtctggacctgagttggtgaagcctggggcttcagtgaagatatcctgcaaggttctgactacagcttcactaactcctatataaactgggtgaagcagaggcctggacagggacttgagtggattggatggatttatcctggaagcactaatactaagtataatgagaagttctgggacaggccacattgactgtggacacatcctccaacacagcctacatggagctcaggggtctgacctctgaggactctgcggtctatttctgtgaaatggggactcactatagttacgatgttatggactatggggtcaaggaacctcagtcaccgtctcctca

SEQ ID NO: 200 - DNA, 319 bp, synthetic construct
  misc_feature 1..319: note = DNA sequence of light chain variable region of monoclonal antibody 179 9E12
  source 1..319: mol_type = other DNA
gacattgtgatgaccccgtctcaaaaattcatgtccacatcagtaggagacagggtcagcatcacctcaaggccagtcagaatgttcgtacttctgtaggctggtatcaacagaaaccagggcagtctcctaaaccactgatttacgcgacatccaaccggcacactggagtccctgatcgcttcacaggcagtgatctgggacagatttcagtctcaccattaccaatgtgcaatctgaagaccaggcagattatttctgtctgcagcattggaattatccgtggacgttcggtggaggcaccaagctggaaatcaaa

SEQ ID NO: 201 - protein, 138 aa, synthetic construct
  REGION 1..138: note = Amino acid sequence of heavy chain of monoclonal antibody 179B 10A7
MGWSWIFLFLLSGTAGVHCQIQLQQSGPELVKPGASVKISCKASDYSFTKSYINWVKQRPGQGLEWIGWIYPGSANIKYNEKFRDKATLTVDTSSNTAYIQLSSLTSEDSAVYFCEMGTHYTYDVMDWGQGTSVTVSS

SEQ ID NO: 202 - protein, 5 aa, synthetic construct
  REGION 1..5: note = Amino acid sequence of heavy chain CDR1 of monoclonal antibody 179B 10A7
KSYIN

SEQ ID NO: 203 - protein, 17 aa, synthetic construct
  REGION 1..17: note = Amino acid sequence of heavy chain CDR2 of monoclonal antibody 179B 10A7
WIYPGSANIKYNEKFRD

SEQ ID NO: 204 - protein, 11 aa, synthetic construct
  REGION 1..11: note = Amino acid sequence of heavy chain CDR3 of monoclonal antibody 179B 10A7
GTHYTYDVMDY

SEQ ID NO: 205 - protein, 127 aa, synthetic construct
  REGION 1..127: note = Amino acid sequence of light chain of monoclonal antibody 179B 10A7
MEFQTQVFVFVLLWLSGVDGDIVMTPSQKFMSTSVGDRVSITCKASQNVRTSVGWYQQKPGQSPKPLIYAASNRHTGVPDRFTGSGSGTDFSLTITNVQSEDQADYFCLQHWNYPWTFGGGTKLEIK

SEQ ID NO: 206 - protein, 11 aa, synthetic construct
  REGION 1..11: note = Amino acid sequence of light chain CDR1 of monoclonal antibody 179B 10A7
KASQNVRTSVG

SEQ ID NO: 207 - protein, 7 aa, synthetic construct
  REGION 1..7: note = Amino acid sequence of light chain CDR2 of monoclonal antibody 179B 10A7
AASNRHT

SEQ ID NO: 208 - protein, 9 aa, synthetic construct
  REGION 1..9: note = Amino acid sequence of light chain CDR3 of monoclonal antibody 179B 10A7
LQHWNYPWT

SEQ ID NO: 209 - DNA, 414 bp, synthetic construct
  misc_feature 1..414: note = DNA sequence of heavy chain of monoclonal antibody 179B 10A7
  source 1..414: mol_type = other DNA
atgggatggagctggatctttcttttcctcctgtcaggaactgcaggtgtccattgccagatccagctgcagcagtctggacctgagttggtgaagcctggggcttcagtgaagatatcctgcaaggttctgactacagcttcactaagtcctatataaactgggtgaagcagaggcctggacagggacttgagtggattggatggatttatcctggaagcgcaaatattaaatataatgagaagttcagggacaggccacattgactgtagacacatcctccaacacagcctacatacaactcagcagtctgacctctgaggactctgcggtctatttctgtgaaatggggactcactatacttacgatgttatggactatggggtcaaggaacctcagtcaccgtctcctca

SEQ ID NO: 210 - DNA, 15 bp, synthetic construct
  misc_feature 1..15: note = DNA sequence of heavy chain CDR1 of monoclonal antibody 179B 10A7
  source 1..15: mol_type = other DNA
aagtcctatataaac

SEQ ID NO: 211 - DNA, 51 bp, synthetic construct
  misc_feature 1..51: note = DNA sequence of heavy chain CDR2 of monoclonal antibody 179B 10A7
  source 1..51: mol_type = other DNA
tggatttatcctggaagcgcaaatattaaatataatgagaagttcagggac

SEQ ID NO: 212 - DNA, 33 bp, synthetic construct
  misc_feature 1..33: note = DNA sequence of heavy chain CDR3 of monoclonal antibody 179B 10A7
  source 1..33: mol_type = other DNA
gggactcactatacttacgatgttatggactac

SEQ ID NO: 213 - DNA, 379 bp, synthetic construct
  misc_feature 1..379: note = DNA sequence of light chain of monoclonal antibody 179B 10A7
  source 1..379: mol_type = other DNA
atggagtttcagacccaggtctttgtattcgtgttgctctggttgtctggtgttgatggagacattgtgatgaccccgtctcaaaaattcatgtccacatcagttggagacagggtcagcatcaccttaaggccagtcagaatgttcgtacttctgtaggctggtatcaacagaaaccagggcagtctcctaaaccactgatttacgcggcatccaaccggcacactggagtccctgatcgcttcacaggcagtgatctgggacagacttcagtctcaccattaccaatgtgcaatctgaagaccaggcagattatttctgtctgcaacattggaattatccgtggacgttcggtggaggcaccaagctggaaatcaaa

SEQ ID NO: 214 - DNA, 33 bp, synthetic construct; identical to SEQ ID NO: 194
  misc_feature 1..33: note = DNA sequence of light chain CDR1 of monoclonal antibody 179B 10A7
  source 1..33: mol_type = other DNA

SEQ ID NO: 215 - DNA, 21 bp, synthetic construct
  misc_feature 1..21: note = DNA sequence of light chain CDR2 of monoclonal antibody 179B 10A7
  source 1..21: mol_type = other DNA
gcggcatccaaccggcacact

SEQ ID NO: 216 - DNA, 27 bp, synthetic construct
  misc_feature 1..27: note = DNA sequence of light chain CDR3 of monoclonal antibody 179B 10A7
  source 1..27: mol_type = other DNA
ctgcaacattggaattatccgtggacg

SEQ ID NO: 217 - protein, 119 aa, synthetic construct
  REGION 1..119: note = Amino acid sequence of heavy chain variable region of monoclonal antibody 179B 10A7
QIQLQQSGPELVKPGASVKISCKASDYSFTKSYINWVKQRPGQGLEWIGWIYPGSANIKYNEKFRDKATLTVDTSSNTAYIQLSSLTSEDSAVYFCEMGTHYTYDVMDWGQGTSVTVSS

SEQ ID NO: 218 - protein, 107 aa, synthetic construct
  REGION 1..107: note = Amino acid sequence of light chain variable region of monoclonal antibody 179B 10A7
DIVMTPSQKFMSTSVGDRVSITCKASQNVRTSVGWYQQKPGQSPKPLIYAASNRHTGVPDRFTGSGSGTDFSLTITNVQSEDQADYFCLQHWNYPWTFGGGTKLEIK

SEQ ID NO: 219 - DNA, 357 bp, synthetic construct
  misc_feature 1..357: note = DNA sequence of heavy chain variable region of monoclonal antibody 179B 10A7
  source 1..357: mol_type = other DNA
cagatccagctgcagcagtctggacctgagttggtgaagcctggggcttcagtgaagatatcctgcaaggttctgactacagcttcactaagtcctatataaactgggtgaagcagaggcctggacagggacttgagtggattggatggatttatcctggaagcgcaaatattaaatataatgagaagttcagggacaggccacattgactgtagacacatcctccaacacagcctacatacaactcagcagtctgacctctgaggactctgcggtctatttctgtgaaatggggactcactatacttacgatgttatggactatggggtcaaggaacctcagtcaccgtctcctca

SEQ ID NO: 220 - DNA, 319 bp, synthetic construct
  misc_feature 1..319: note = DNA sequence of light chain variable region of monoclonal antibody 179B 10A7
  source 1..319: mol_type = other DNA
gacattgtgatgaccccgtctcaaaaattcatgtccacatcagttggagacagggtcagcatcaccttaaggccagtcagaatgttcgtacttctgtaggctggtatcaacagaaaccagggcagtctcctaaaccactgatttacgcggcatccaaccggcacactggagtccctgatcgcttcacaggcagtgatctgggacagacttcagtctcaccattaccaatgtgcaatctgaagaccaggcagattatttctgtctgcaacattggaattatccgtggacgttcggtggaggcaccaagctggaaatcaaa

SEQ ID NO: 221 - protein, 135 aa, synthetic construct
  REGION 1..135: note = Amino acid sequence of heavy chain of monoclonal antibody 179A 6H6
MYLGLNCVIIVFLLKGVQSEVKLEESGGGLVPPGGSMKLSCSVSGFTFSDAWIDWVRQSPEKGLEWIAESRNKANNYAIYYTESVKGRFTISRDDSKSRVYLQMNNLRVEDTGIYYCTGPDYDSYWGGTLVTVSA

SEQ ID NO: 222 - protein, 5 aa, synthetic construct
  REGION 1..5: note = Amino acid sequence of heavy chain CDR1 of monoclonal antibody 179A 6H6
DAWID

SEQ ID NO: 223 - protein, 19 aa, synthetic construct
  REGION 1..19: note = Amino acid sequence of heavy chain CDR2 of monoclonal antibody 179A 6H6
ESRNKANNYAIYYTESVKG

SEQ ID NO: 224 - protein, 6 aa, synthetic construct
  REGION 1..6: note = Amino acid sequence of heavy chain CDR3 of monoclonal antibody 179A 6H6
PDYDSY

SEQ ID NO: 225 - protein, 127 aa, synthetic construct
  REGION 1..127: note = Amino acid sequence of light chain of monoclonal antibody 179A 6H6
MEFQTQVFVFVLLWLSGVDGDVVMTQSQKIMSTSVGDRVSITCKASQNVRTAVTWYQLKPGQSPKALIYLASNRHTGVPDRFRGSGSGTDFTLTIINVQSEDLADYFCLQHWNYPLTFGAGTKLELK

SEQ ID NO: 226 - protein, 11 aa, synthetic construct
  REGION 1..11: note = Amino acid sequence of light chain CDR1 of monoclonal antibody 179A 6H6
KASQNVRTAVT

SEQ ID NO: 227 - protein, 7 aa, synthetic construct
  REGION 1..7: note = Amino acid sequence of light chain CDR2 of monoclonal antibody 179A 6H6
LASNRHT

SEQ ID NO: 228 - protein, 9 aa, synthetic construct
  REGION 1..9: note = Amino acid sequence of light chain CDR3 of monoclonal antibody 179A 6H6
LQHWNYPLT

SEQ ID NO: 229 - DNA, 405 bp, synthetic construct
  misc_feature 1..405: note = DNA sequence of heavy chain of monoclonal antibody 179A 6H6
  source 1..405: mol_type = other DNA
atgtacttgggactgaactgtgtaatcatagtttttctcttaaaaggtgtccagagtgaagtgaagcttgaggagtctggaggaggcttggtgccacctggaggatccatgaaactctcttgttctgttctggattcacttttagtgatgcctggatagactgggtccgccagtctccagagaaggggcttgagtggattgctgaaagtcgaaacaaagctaataattatgcaatatattacactgagtctgtgaagggcggttcaccatctcaagagatgattccaaaagtagagtgtacctgcaaatgaacaacttaagagttgaagacactggcatttattactgtaccgggccggattacgactcttactggggccagggactctggtcactgtctctgca

SEQ ID NO: 230 - DNA, 15 bp, synthetic construct
  misc_feature 1..15: note = DNA sequence of heavy chain CDR1 of monoclonal antibody 179A 6H6
  source 1..15: mol_type = other DNA
gatgcctggatagac

SEQ ID NO: 231 - DNA, 56 bp, synthetic construct
  misc_feature 1..56: note = DNA sequence of heavy chain CDR2 of monoclonal antibody 179A 6H6
  source 1..56: mol_type = other DNA
gaaagtcgaaacaaagctaataattatgcaatatattacactgagtctgtgaaggg

SEQ ID NO: 232 - DNA, 18 bp, synthetic construct
  misc_feature 1..18: note = DNA sequence of heavy chain CDR3 of monoclonal antibody 179A 6H6
  source 1..18: mol_type = other DNA
ccggattacgactcttac

SEQ ID NO: 233 - DNA, 379 bp, synthetic construct
  misc_feature 1..379: note = DNA sequence of light chain of monoclonal antibody 179A 6H6
  source 1..379: mol_type = other DNA
atggagtttcagacccaggtctttgtattcgtgttgctctggttgtctggtgttgatggagacgttgtgatgacccagtctcaaaaaatcatgtccacatcagtaggagacagggtcagcatcacctcaaggccagtcagaatgttcgtactgctgttacctggtatcaactgaaaccaggtcagtctcctaaagcactgatttacttggcatccaaccggcacactggagtccctgatcgctttagaggcagtgatctgggacagatttcactcttaccattatcaatgtgcaatctgaggacctggcagattatttctgtctgcaacattggaattatcctctcacgttcggtgctgggaccaagctggagttgaaa

SEQ ID NO: 234 - DNA, 33 bp, synthetic construct
  misc_feature 1..33: note = DNA sequence of light chain CDR1 of monoclonal antibody 179A 6H6
  source 1..33: mol_type = other DNA
aaggccagtcagaatgttcgtactgctgttacc

SEQ ID NO: 235 - DNA, 21 bp, synthetic construct
  misc_feature 1..21: note = DNA sequence of light chain CDR2 of monoclonal antibody 179A 6H6
  source 1..21: mol_type = other DNA
ttggcatccaaccggcacact

SEQ ID NO: 236 - DNA, 27 bp, synthetic construct
  misc_feature 1..27: note = DNA sequence of light chain CDR3 of monoclonal antibody 179A 6H6
  source 1..27: mol_type = other DNA
ctgcaacattggaattatcctctcacg

SEQ ID NO: 237 - protein, 116 aa, synthetic construct
  REGION 1..116: note = Amino acid sequence of heavy chain variable region of monoclonal antibody 179A 6H6
EVKLEESGGGLVPPGGSMKLSCSVSGFTFSDAWIDWVRQSPEKGLEWIAESRNKANNYAIYYTESVKGRFTISRDDSKSRVYLQMNNLRVEDTGIYYCTGPDYDSYWGGTLVTVSA

SEQ ID NO: 238 - protein, 107 aa, synthetic construct
  REGION 1..107: note = Amino acid sequence of light chain variable of monoclonal antibody 179A 6H6
DVVMTQSQKIMSTSVGDRVSITCKASQNVRTAVTWYQLKPGQSPKALIYLASNRHTGVPDRFRGSGSGTDFTLTIINVQSEDLADYFCLQHWNYPLTFGAGTKLELK

SEQ ID NO: 239 - DNA, 278 bp, synthetic construct
  misc_feature 1..278: note = DNA sequence of heavy chain variable region of monoclonal antibody 179A 6H6
  source 1..278: mol_type = other DNA
ttctggattcacttttagtgatgcctggatagactgggtccgccagtctccagagaaggggcttgagtggattgctgaaagtcgaaacaaagctaataattatgcaatatattacactgagtctgtgaagggcggttcaccatctcaagagatgattccaaaagtagagtgtacctgcaaatgaacaacttaagagttgaagacactggcatttattactgtaccgggccggattacgactcttactggggccagggactctggtcactgtctctgca

SEQ ID NO: 240 - DNA, 319 bp, synthetic construct
  misc_feature 1..319: note = DNA sequence of light chain variable region of monoclonal antibody 179A 6H6
  source 1..319: mol_type = other DNA
gacgttgtgatgacccagtctcaaaaaatcatgtccacatcagtaggagacagggtcagcatcacctcaaggccagtcagaatgttcgtactgctgttacctggtatcaactgaaaccaggtcagtctcctaaagcactgatttacttggcatccaaccggcacactggagtccctgatcgctttagaggcagtgatctgggacagatttcactcttaccattatcaatgtgcaatctgaggacctggcagattatttctgtctgcaacattggaattatcctctcacgttcggtgctgggaccaagctggagttgaaa

SEQ ID NO: 241 - protein, 137 aa, synthetic construct
  REGION 1..137: note = Amino acid sequence of heavy chain of monoclonal antibody 179B 9E7
MYLGLNCVIIVFLLKGVQSEMKLEESGGGLVQPGGSMKLSCTASGFTFSDAWMDWVRQSPEKGLEWVAEIRNKAYNHATYYAESVRGRFTISRDDSKSKMFLEMNSLRPEDTGIYYCIGGIWTDFDYGQGTTLTVSS

SEQ ID NO: 242 - protein, 5 aa, synthetic construct
  REGION 1..5: note = Amino acid sequence of heavy chain CDR1 of monoclonal antibody 179B 9E7
DAWMD

SEQ ID NO: 243 - protein, 19 aa, synthetic construct
  REGION 1..19: note = Amino acid sequence of heavy chain CDR2 of monoclonal antibody 179B 9E7
EIRNKAYNHATYYAESVRG

SEQ ID NO: 244 - protein, 8 aa, synthetic construct
  REGION 1..8: note = Amino acid sequence of heavy chain CDR3 of monoclonal antibody 179B 9E7
GIWTDFDY

SEQ ID NO: 245 - protein, 127 aa, synthetic construct
  REGION 1..127: note = Amino acid sequence of light chain of monoclonal antibody 179B 9E7
MESQTQVFVYMLLWLSGVDGDIVMTQSQKFMSTSVGDRVSVTCKASQNVGINVAWYQQKSGQPPKALIYSASSRYSGVPDRFTGSGSGTDFTLTISNVQSEDLAEYFCHQYNNYPLTFGAGTKLDLK

SEQ ID NO: 246 - protein, 11 aa, synthetic construct
  REGION 1..11: note = Amino acid sequence of light chain CDR1 of monoclonal antibody 179B 9E7
KASQNVGINVA

SEQ ID NO: 247 - protein, 7 aa, synthetic construct
  REGION 1..7: note = Amino acid sequence of light chain CD2 of monoclonal antibody 179B 9E7
SASSRYS

SEQ ID NO: 248 - protein, 9 aa, synthetic construct
  REGION 1..9: note = Amino acid sequence of light chain CDR3 of monoclonal antibody 179B 9E7
HQYNNYPLT

SEQ ID NO: 249 - DNA, 411 bp, synthetic construct
  misc_feature 1..411: note = DNA sequence of heavy chain of monoclonal antibody 179B 9E7
  source 1..411: mol_type = other DNA
atgtacttgggactgaactgtgtaatcatagtttttctcttaaaaggtgtccagagtgaaatgaagcttgaggagtctggaggaggcttagtgcaacctggaggatccatgaaactctcctgtactgttctggattcacttttagtgatgcctggatggactgggtccgccagtctccagagaaggggcttgagtgggttgctgagattagaaacaaagcttataatcatgcaacatattatgctgagtctgtggagggaggttcaccatctccagagatgattccaaaagtaaaatgttcctggaaatgaacagcttaagacctgaagacactggcatttattattgtatcggggggatttggacggactttgactactgggccaaggcaccactctcacagtctcctca

SEQ ID NO: 250 - DNA, 15 bp, synthetic construct
  misc_feature 1..15: note = DNA sequence of heavy chain CDR1 of monoclonal antibody 179B 9E7
  source 1..15: mol_type = other DNA
gatgcctggatggac

SEQ ID NO: 251 - DNA, 56 bp, synthetic construct
  misc_feature 1..56: note = DNA sequence of heavy chain CDR2 of monoclonal antibody 179B 9E7
  source 1..56: mol_type = other DNA
gagattagaaacaaagcttataatcatgcaacatattatgctgagtctgtggaggg

SEQ ID NO: 252 - DNA, 24 bp, synthetic construct
  misc_feature 1..24: note = DNA sequence of heavy chain CDR3 of monoclonal antibody 179B 9E7
  source 1..24: mol_type = other DNA
gggatttggacggactttgactac

SEQ ID NO: 253 - DNA, 379 bp, synthetic construct
  misc_feature 1..379: note = DNA sequence of light chain of monoclonal antibody 179B 9E7
  source 1..379: mol_type = other DNA
atggagtcacagactcaggtctttgtatacatgttgctgtggttgtctggtgttgatggagacattgtgatgacccagtctcaaaaattcatgtccacatcagtaggagacagggtcagcgtcacctcaaggccagtcagaatgtgggtattaatgtagcctggtatcaacagaaatcagggcaacctcctaaagcactgatttattcggcatcttcccggtacagtggagtccctgatcgcttcacaggcagtgatctgggacagatttcactctcaccatcagcaatgtgcagtctgaagacttggcagagtatttctgtcaccaatataataattatcctctcacgttcggtgctgggaccaagctggacctgaaa

SEQ ID NO: 254 - DNA, 33 bp, synthetic construct
  misc_feature 1..33: note = DNA sequence of light chain CDR1 of monoclonal antibody 179B 9E7
  source 1..33: mol_type = other DNA
aaggccagtcagaatgtgggtattaatgtagcc

SEQ ID NO: 255 - DNA, 21 bp, synthetic construct
  misc_feature 1..21: note = DNA sequence of light chain CDR2 of monoclonal antibody 179B 9E7
  source 1..21: mol_type = other DNA
tcggcatcttcccggtacagt

SEQ ID NO: 256 - DNA, 27 bp, synthetic construct
  misc_feature 1..27: note = DNA sequence of light chain CDR3 of monoclonal antibody 179B 9E7
  source 1..27: mol_type = other DNA
caccaatataataattatcctctcacg

SEQ ID NO: 257 - protein, 118 aa, synthetic construct
  REGION 1..118: note = Amino acid sequence of heavy chain variable region of monoclonal antibody 179B 9E7
EMKLEESGGGLVQPGGSMKLSCTASGFTFSDAWMDWVRQSPEKGLEWVAEIRNKAYNHATYYAESVRGRFTISRDDSKSKMFLEMNSLRPEDTGIYYCIGGIWTDFDYGQGTTLTVSS

SEQ ID NO: 258 - protein, 107 aa, synthetic construct
  REGION 1..107: note = Amino acid sequence of light chain variable region of monoclonal antibody 179B 9E7
DIVMTQSQKFMSTSVGDRVSVTCKASQNVGINVAWYQQKSGQPPKALIYSASSRYSGVPDRFTGSGSGTDFTLTISNVQSEDLAEYFCHQYNNYPLTFGAGTKLDLK

SEQ ID NO: 259 - DNA, 354 bp, synthetic construct
  misc_feature 1..354: note = DNA sequence of heavy chain variable region of monoclonal antibody 179B 9E7
  source 1..354: mol_type = other DNA
gaaatgaagcttgaggagtctggaggaggcttagtgcaacctggaggatccatgaaactctcctgtactgttctggattcacttttagtgatgcctggatggactgggtccgccagtctccagagaaggggcttgagtgggttgctgagattagaaacaaagcttataatcatgcaacatattatgctgagtctgtggagggaggttcaccatctccagagatgattccaaaagtaaaatgttcctggaaatgaacagcttaagacctgaagacactggcatttattattgtatcggggggatttggacggactttgactactgggccaaggcaccactctcacagtctcctca

SEQ ID NO: 260 - DNA, 319 bp, synthetic construct
  misc_feature 1..319: note = DNA sequence of light chain variable region of monoclonal antibody 179B 9E7
  source 1..319: mol_type = other DNA
gacattgtgatgacccagtctcaaaaattcatgtccacatcagtaggagacagggtcagcgtcacctcaaggccagtcagaatgtgggtattaatgtagcctggtatcaacagaaatcagggcaacctcctaaagcactgatttattcggcatcttcccggtacagtggagtccctgatcgcttcacaggcagtgatctgggacagatttcactctcaccatcagcaatgtgcagtctgaagacttggcagagtatttctgtcaccaatataataattatcctctcacgttcggtgctgggaccaagctggacctgaaa